(19) United States
(12) Patent Application Publication
(10) Pub. No.: US 2007/0072859 A1
(43) Pub. Date: Mar. 29, 2007

(54) Pharmaceutical compounds

(75) Inventors: Serge Louis Boulet, Fishers, IN (US); Anette Margareta Johansson, Indianapolis, IN (US); Sarah Marie Smith, Indianapolis, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company, Indianapolis, IN (US)

(21) Appl. No.: 10/597,835

(22) PCT Filed: Feb. 18, 2005
(86) PCT No.: PCT/US05/05226

§ 371 Date: Aug. 9, 2006

Related U.S. Application Data
(60) Provisional application No. 60/550,259, filed on Mar. 5, 2004.

(51) Int. Cl. A61K 31/5375; A61K 31/445; A61K 31/40; A61K 31/137; C07D 211/06

(52) U.S. Cl. 514/237.8; 514/317; 514/408; 514/651; 514/649; 548/561; 546/205; 546/229; 544/158; 544/162; 564/341; 564/349

(57) ABSTRACT

The present invention relates to inhibitors of serotonin and/or norepinephrine reuptake and specifically provides compounds of formula (I): wherein A is selected from —O— and —S—; X is selected from C₁-C₈ alkyl, C₂-C₈ alkenyl, and C₄-C₈ cycloalkylalkyl, each of which may be optionally substituted with up to 3 substituents each independently selected from phenyl, pyrrolidinyl, piperidinyl, morpholinyl, halo, C₁-C₄ alkyl, C₁-C₄ alkoxy, C₁-C₄ alkyl-S(O)ₙ— where n is 0, 1 or 2, —CF₃, —CN and —CONH₂; Y is selected from (a), (b), (c), (d), (e), (f) where R₃, R₄ and R₅ are independently selected from hydrogen, halo, C₁-C₄ alkyl, C₁-C₄ alkoxy, C₁-C₄ alkyl-S(O)ₙ— where n is 0, 1 or 2, nitro, acetyl, —CF₃, —SCF₃ and cyano; R₆ and R₇ are independently selected from halo, C₁-C₄ alkyl, C₁-C₄ alkoxy, C₁-C₄ alkyl-S(O)ₙ— where n is 0, 1 or 2, nitro, acetyl, —CF₃, —SCF₃ and cyano; R₈ is selected from chloro, bromo, iodo, C₁-C₄alkyl, C₁-C₄ alkoxy, C₁-C₄ alkyl-S(O)ₙ— where n is 0, 1 or 2, nitro, acetyl, —CF₃, —SCF₃ and cyano; R₁ and R₂ are each independently hydrogen or C₁-C₄ alkyl; or pharmaceutically acceptable salts thereof; or compositions thereof and methods of using the same.

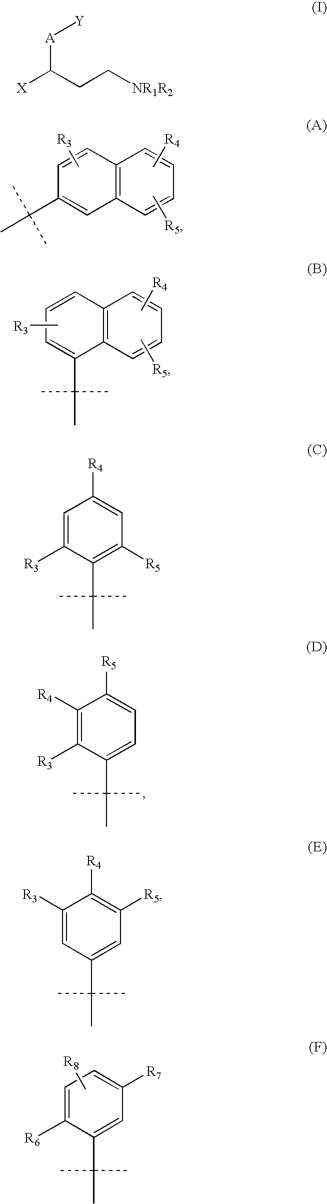

This invention relates to 3-aryloxy/thio-3-substituted propanamines, and to their use in inhibiting serotonin and norepinephrine reuptake.

### BACKGROUND OF THE INVENTION

Serotonin (5HT) has been implicated in the aetiology of many disease states and has been found to be of importance in mental illnesses, depression, anxiety, schizophrenia, eating disorders, obsessive compulsive disorder (OCD) and migraine. Indeed many currently used treatments of these disorders are thought to act by modulating serotonergic tone. During the last decade, multiple serotonin receptor subtypes have been characterized. This has led to the realization that many treatments act via the serotonergic system, such as selective serotonin reuptake inhibitor (SSRI) antidepressants which increase serotonin transmission, such as, for example, the hydrochloride salt of fluoxetine.

Drugs that exert their main action on the norepinephrinergic system have been available for some time, however their lack of selectivity made it difficult to determine specific clinical effects produced by a selective action on norepinephrine reuptake. Accumulating evidence indicates that the norepinephrinergic system modulates drive and energy, whereas the serotonergic system modulates mood. Thus norepinephrine appears to play an important role in the disturbances of vegetative function associated with affective, anxiety and cognitive disorders. Atomoxetine hydrochloride is a selective inhibitor of norepinephrine, and is currently marketed for the treatment of attention deficit hyperactivity disorder (ADHD).

Norepinephrine and serotonin receptors are known to interact anatomically and pharmacologically. Compounds that affect only serotonin have been shown to exhibit modulatory effects on norepinephrine, pointing toward an important relationship between the two neurotransmitter systems.

Duloxetine, (+)-N-methyl-3-(1-naphthalenyloxy)-2-thiophenepropanamine hydrochloride, inhibits the reuptake of both norepinephrine and serotonin, and is currently under development for the treatment of depression and urinary incontinence. The compound duloxetine was disclosed in U.S. Pat. Nos. 5,023,269 and 4,956,388.

U.S. Pat. No. 4,018,895 describes aryloxyphenyl propanamine compounds including compounds of the formula

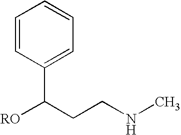

Where R is, for example, phenyl, substituted phenyl, tolyl or anisyl. The compounds block the uptake of various physiologically active monoamines including serotonin, norepinephrine and dopamine. Some of the compounds are selective to one of the monoamines and others have multiple activity. The compounds are indicated as psychotropic agents. Some also antagonize the effects of apomorphine and/or reserpine.

WO 00/02551 describes inter alia 3-aryloxy-3-substituted propanamines which are active at the NMDA receptor and serotonin reuptake site.

WO 96/09288 describes indole derivatives which are active at the 5HT receptor. The 5-membered ring portion of the indole moiety is further substituted by one of a number of amine functional groups.

WO 01/62714 discloses phenylheteroalkylamine derivatives which are inhibitors of nitric oxide synthase. WO 03/011831 discloses heteroarylheteroalkylamine derivatives which are inhibitors of nitric oxide synthase.

WO 02/094262 discloses heteroaryloxy 3-substituted propanamines as serotonin and norepinephrine reuptake inhibitors.

The present invention provides novel 3-aryloxy/thio-3-substituted propanamines which are inhibitors of both serotonin and norepinephrine reuptake. Compounds of the present invention may exihibit (i) greater potency of inhibition of the serotonin and/or norepinephrine transporters, and/or (ii) improved selectivity of inhibition of the serotonin and/or norepinephrine transporters relative to the dopamine transporter, and/or (iii) improved ADME properties (e.g. reduced tendency to act as a substrate and/or inhibitor for the enzyme Cytochrome P450 2D6), and/or (iv) improved acid stability, as compared to known inhibitors of both serotonin and norepinephrine reuptake.

### SUMMARY OF THE INVENTION

According to the present invention there is provided a compound of formula I:

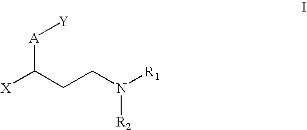

where

A is selected from —O— and —S—;

X is selected from C₁-C₈ alkyl, C₂-C₈ alkenyl, and C₄-C₈ cycloalkylalkyl, each of which may be optionally substituted with up to 3 substituents each independently selected from phenyl, pyrrolidinyl, piperidinyl, morpholinyl, halo, C₁-C₄ alkyl, C₁-C₄ alkoxy, C₁-C₄ allyl-S(O)ₙ— where n is 0, 1 or 2, —CF₃, —CN and —CONH₂;

Y is selected from 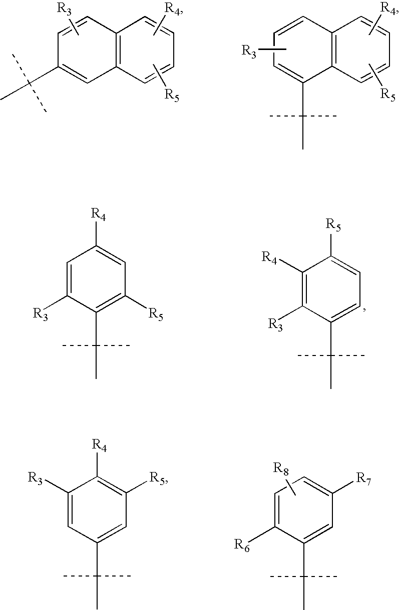
wherein
R₃, R₄ and R₅ are independently selected from hydrogen, halo, C₁-C₄ alkyl, C₁-C₄ alkoxy, C₁-C₄ alkyl-S(O)ₙ— where n is 0, 1 or 2, nitro, acetyl, —CF₃, —SCF₃ and cyano;
R₆ and R₇ are independently selected from halo, C₁-C₄ alkyl, C₁-C₄ alkoxy, C₁-C₄ allyl-S(O)ₙ— where n is 0, 1 or 2, nitro, acetyl, —CF₃, —SCF₃ and cyano;
R₈ is selected from chloro, bromo, iodo, C₁-C₄ alkyl, C₁-C₄ alkoxy, C₁-C₄ alkyl-S(O)ₙ— where n is 0, 1 or 2, nitro, acetyl, —CF₃, —SCF₃ and cyano;

R₁ and R₂ are each independently hydrogen or C₁-C₄ alkyl;

or pharmaceutically acceptable salts thereof.

It will be appreciated that a compound of formula I will possess at least one asymmetric or chiral center. Where a structural formula does not specify the stereochemistry at one or more chiral centers, it encompasses all possible stereoisomers and all possible mixtures of stereoisomers (including, but not limited to, mixtures of enantiomers and/or diastereomers). The skilled artisan will recognize compounds of the invention may exist in and be isolated in enantiomerically pure form, in racemic form, in a diastereoisomeric mixture or as a single diastereomeric form.

In one embodiment of the present invention, the compound possesses the stereochemistry defined in formula II

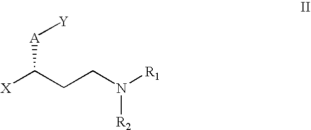

In another embodiment of the present invention, the compound possesses the stereochemistry defined in formula III

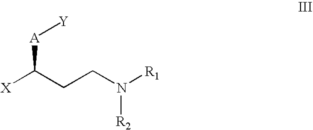

In addition to the compounds of formula I the present invention further provides pharmaceutical compositions comprising a compound of formula I or a pharmaceutically acceptable salt thereof, together with a pharmaceutically acceptable diluent or carrier.

Further features of the invention include processes useful for the manufacture of a compound of formula I as defined above.

In another embodiment of the present invention, compounds of formula I are inhibitors, of the reuptake of serotonin and norepinephrine and as such are useful for the treatment of disorders associated with serotonin and norepinephrine dysfunction. Such disorders include depression, OCD, anxiety, memory loss, urinary incontinence, conduct disorders, ADHD, obesity, alcoholism, smoking cessation, hot flashes/flushes and pain.

### DETAILED DESCRIPTION

In the present specification the term “C₁-C₈ alkyl” means a monovalent unsubstituted saturated straight-chain or branched-chain hydrocarbon radical having from 1 to 8 carbon atoms and includes methyl, ethyl, propyl, iso-propyl, butyl, iso-butyl, sec-butyl, t-butyl, pentyl, hexyl, heptyl, octyl, and the like. Likewise, the term “C₁-C₄ alkyl” means a monovalent unsubstituted saturated straight-chain or branched-chain hydrocarbon radical having from 1 to 4 carbon atoms and includes methyl, ethyl, propyl, iso-propyl, butyl, iso-butyl, sec-butyl, and t-butyl.

In the present specification the term “C₂-C₈ alkenyl” means a monovalent unsubstituted unsaturated straight-chain or branched-chain hydrocarbon radical having from 2 to 8 carbon atoms and one or more carbon-carbon double bonds, and includes ethylene, propylene, iso-propylene, butylene, iso-butylene, sec-butylene, pentylene, hexylene, heptylene, octylene, and the like.

In the present specification the term “C₄-C₈ cycloalkylalkyl” means a monovalent unsubstituted saturated cyclic hydrocarbon radical having from 3 to 7 carbon atoms linked to the point of substitution by a divalent unsubstituted saturated straight-chain or branched-chain hydrocarbon radical having at least l carbon atom and includes cyclopropane-methyl, cyclopropane-2-ethyl, cyclobutane-methyl, cyclobutane-2-ethyl, cyclopentane-methyl, cyclopentane-2-ethyl cyclohexane-methyl, cyclohexane-2-ethyl, cycloheptane-methyl, and the like.

In the present specification the term “halo” or “halogen” means fluoro, chloro, bromo or iodo.

In the present specification the term “C₁-C₄ alkoxy” means a monovalent unsubstituted saturated straight-chain or branched-chain hydrocarbon radical having from 1 to 4 carbon atoms linked to the point of substitution by an O atom, and includes methoxy, ethoxy, propoxy, iso-propoxy, butoxy, iso-butoxy, sec-butoxy, and t-butoxy.

In the present specification the term “C₁-C₄ alkyl-S(O)ₙ— where n is 0, 1 or 2” means a monovalent saturated straight-chain or branched-chain hydrocarbon radical linked to a divalent sulfur atom in which the sulfur atom can optionally be oxidized to the sulfone or sulfoxide, and includes methane sulfidyl, methane sulfinyl, methane sulfonyl, ethane-2-sulfidyl, ethane-2-sulfinyl, ethane-2-sulfonyl, propyl-3-sulfidyl, propyl-3-sulfinyl, propyl-3-sulfonyl, isopropyl-2-sulfonyl, butyl-4-sulfidyl, butyl-4-sulfinyl, butyl-4-sulfonyl, isobutyl-3-sulfonyl, t-butyl-2-sulfonyl, and the like.

In the present specification the terms

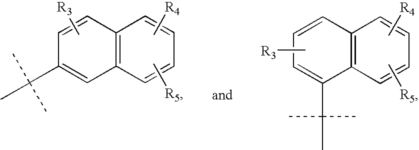

mean a naphthyl ring which R₃, R₄ and R₅ are independently bonded to an available point of attachment and include

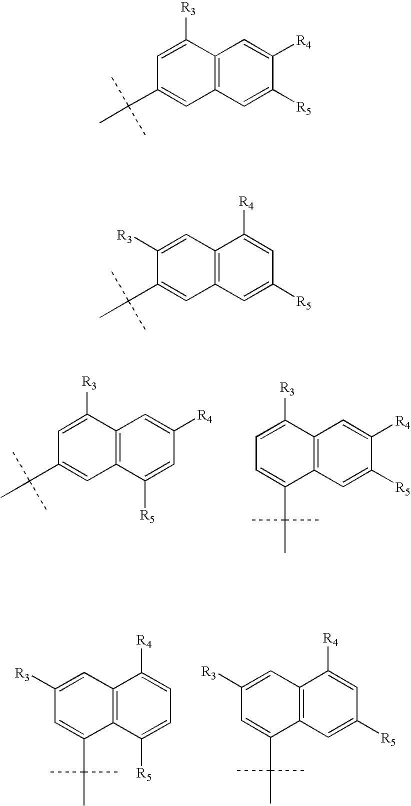

and the like.

In the present specification the terms “pyrrolidinyl”, “piperidinyl” and “morpholinyl” mean a heterocylic ring of the formula

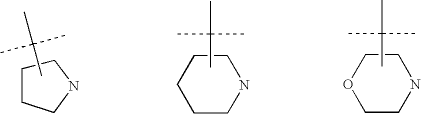

respectively which are independently bonded to an available point of attachment and include

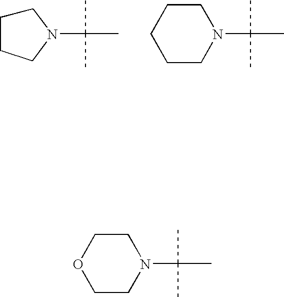

and the like.

In the present specification the term “protecting group,” defined herein as Pg, refers to those groups intended to protect or block functional groups against undesirable reactions during synthetic procedures. In the case of protection of an amine functional group, the suitable protecting group used will depend upon the conditions that will be employed in subsequent reaction steps wherein protection is required. Commonly used amine protecting groups are

disclosed in T. W. Greene and P. G. M. Wuts, Protective Groups In Organic Synthesis, 3rd Ed. (John Wiley & Sons, New York (1999)). Suitable amine protecting groups comprise acyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, alpha-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like, carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, alpha, alpha-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; alkyl groups such as benzyl, triphenylmethyl, benzyloxymethyl and the like; and silyl groups such as trimethylsilyl and the like. Preferred amine protecting groups are acetyl, methyloxycarbonyl, benzoyl, pivaloyl, allyloxycarbonyl, t-butylacetyl, benzyl, t-butyloxycarbonyl (Boc) and benzyloxycarbonyl (Cbz).

In the present specification the term “leaving group,” defined herein as Lg, refers to reactive functional groups involved in synthetic transformations, most particularly, nucleophilic substitution reactions. The selection of a suitable leaving group will depend upon the nature of the desired bond to be formed as well as conditions employed in the reaction such as reacting nucleophile, solvent, time and temperature. Commonly used leaving groups are disclosed in F. A. Carey and R. J. Sundberg, Advanced Organic Chemistry, 2nd Ed., (Plenum Press, New York, 1983). Commonly used leaving groups include sulfonate esters such as trifluoromethanesulfonyl (triyl), p-nitrobenzenesulfonyl, p-toluenesulfonyl (tosyl), and methanesulfonyl (mesyl); halogens such as chloro, bromo, iodo, and fluoro; esters such as acetyl and trifluoroacetyl; and the like.

Compounds of the present invention may be made by a process which is analogous to one known in the chemical art for the production of structurally analogous compounds or by a novel process described herein. Such processes useful for the manufacture of a compound of formula I are provided as further features of the invention.

Further embodiments of the invention include a process for preparing the compound of formula I, or a pharmaceutically acceptable salt thereof, comprising 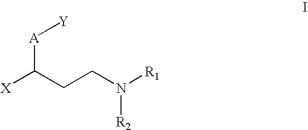
for a compound of formula I where R₂ is hydrogen, deprotecting a compound of formula IV 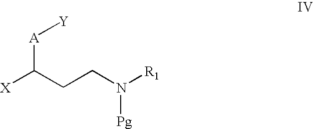
where Pg is an amine protecting group;
whereafter, for the above procedure, when a pharmaceutically acceptable salt of a compound of formula I is required, it is obtained by reacting the basic form of such a compound of formula I with an acid affording a physiologically acceptable counterion, or by any other conventional procedure where the values of X, A, Y, R₁ and R₂ are defined above.

As with any group of pharmaceutically active compounds, some groups are preferred in their end use application. Preferred embodiments of the present invention are given below.

Compounds of formula I wherein X is selected from C₁-C₈ alkyl, and C₄-C₈ cycloalkylalkyl, each of which may be optionally substituted with 1 substituent independently selected from phenyl, pyrrolidinyl, molpholinyl, C₁-C₄ alkyl, C₁-C₄ alkoxy, C₁-C₄ alkyl-S(O)ₙ— where n is 0, and —CF₃ are preferred.

Compounds of formula I wherein X is selected from cyclopropylmethyl, methyl, ethyl, iso-propyl, neo-pentyl, iso-pentyl, methoxymethyl, ethoxymethyl, iso-propoxymethyl, iso-butoxymethyl, tert-butoxymethyl, iso-propylsulfidylmethyl, phenylmethyl, pyrrolidinylmethyl and morpholinomethyl are more preferred.

Compounds of formula I wherein X is n-propyl are even more preferred.

Compounds of formula I wherein A is O are preferred.

Compounds of formula I wherein R₁ is C₁-C₄ alkyl and R₂ is hydrogen are preferred.

Compounds of formula I wherein R₁ is methyl and R₂ is hydrogen are more preferred.

Compounds of formula I wherein Y is selected from

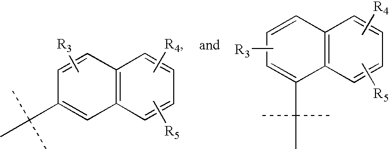

where R₃, R₄ and R₅ are independently selected from hydrogen, halo, C₁-C₄ allyl or —CF₃ are preferred.

Compounds of formula I wherein Y is selected from

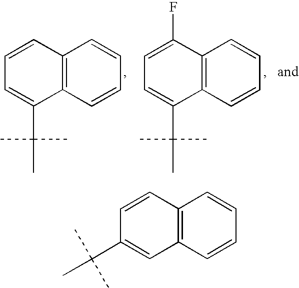

are more preferred.

Compounds of formula I wherein Y is selected from 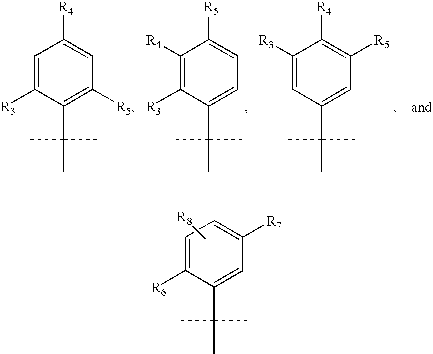
where
R₃, R₄ and R₅ are independently selected from hydrogen, halo, C₁-C₄ alkyl, and —CF₃;
R₆ and R₇ are independently selected from halo, C₁-C₄ allyl, and -CF₃; and
R₈ is selected from chloro, bromo, iodo, C₁-C₄ alkyl, and —CF₃;
provided when R₃ and R₄ are hydrogen, R₅ is not hydrogen

are preferred.

Compounds of formula I wherein Y is selected from

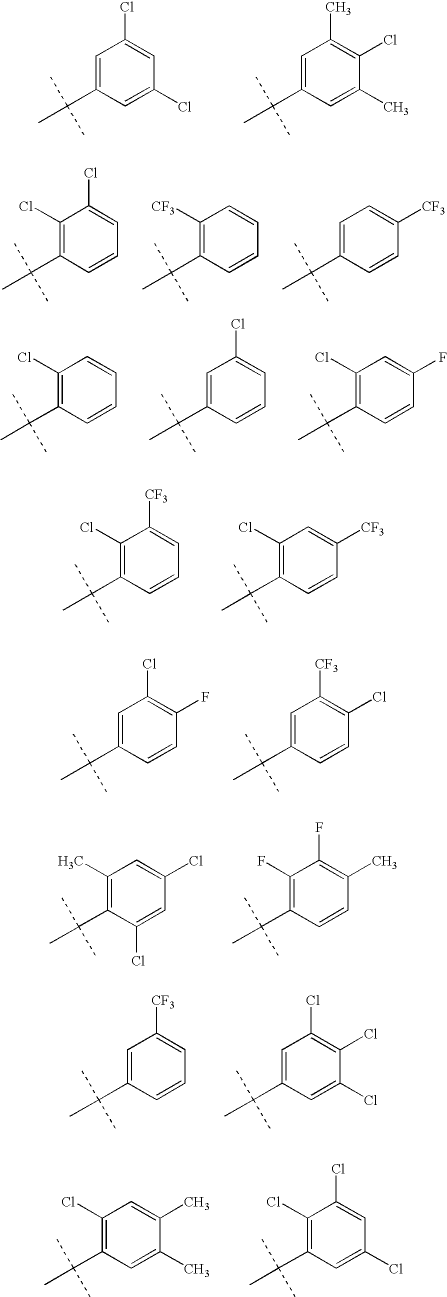

are more preferred.

Compounds of formula I wherein Y is selected from

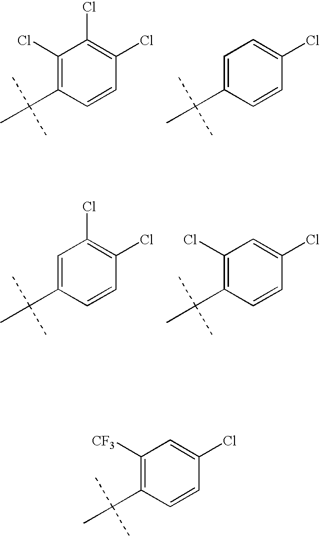

are even more preferred.

Compounds of the present invention may be made by a process which is analogous to one known in the chemical art for the production of structurally analogous compounds or by a novel process described herein. Such processes useful for the manufacture of a compound of formula I as defined above are provided as further features of the invention and are illustrated by the following procedures in which, unless otherwise specified, the meanings of the generic radicals are as defined above.

Generally, a compound of formula I may be prepared from a compound of formula V. A compound of formula V may be prepared from various intermediate compounds such as:

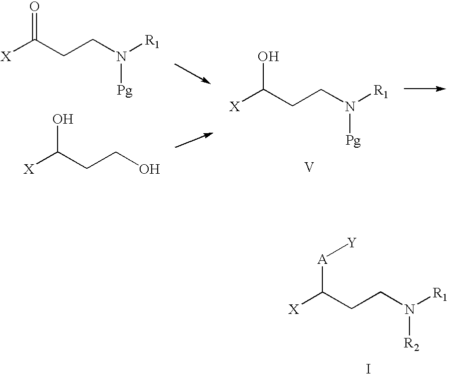

More specifically, a compound of formula V can be prepared via the corresponding 3-amino-N-methoxy-N-methylpropanamide, known as a Weinreb amide, as follows:

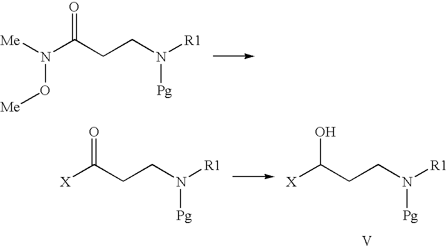

Subjecting a Weinreb amide of N-methyl β-alanine appropriately protected at the nitrogen, for example where Pg is a t-butyl carbamate (Boc) or as a benzyl amine, to an organometallic reagent like an alkyl Grignard or alkyl lithium results in the desired X-substituted ketone. The ketone can be readily reduced to the desired racemic alcohol using standard reducing agents such as sodium borohydride in a protic solvent such as lower order alkyl alcohols.

The Weinreb amides of this invention may be prepared by conventional organic chemistry techniques as exemplified below:

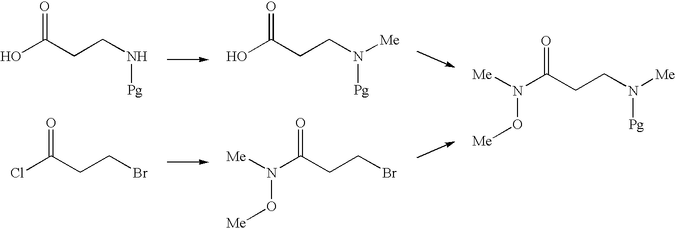

Subjecting a commercial available appropriately N-protected β-alanine to sodium hydride followed by methyl iodide results in the N-methylated derivative, which then can be converted to the Weinreb amide by reaction with N-methyl-O-methylhydroxylamine. The Weinreb amides can also be prepared by reacting a 3-bromopropanoyl chloride with N-methyl-O-methylhydroxylamine to give the Weinreb amide of 3-bromopropanoic acid, which then can be substituted with an appropriately substituted amine to give the desired Weinreb amide.

A compound of formula V may also be prepared by addition of a suitable organometallic reagent to an appropriately N-protected aminoaldehyde. Thus a protected amine where Pg is benzyl can be added to a vinyl aldehyde in a Michael addition reaction to give a 3-aminopropanal. The aminoaldehyde can be subjected to (for example) an alkyl Grignard reagent or an alkyl lithium reagent to give a compound of formula V. The selection of Grignard or organolithium reagents may be used to provide various X substituents for a compound of formula V.

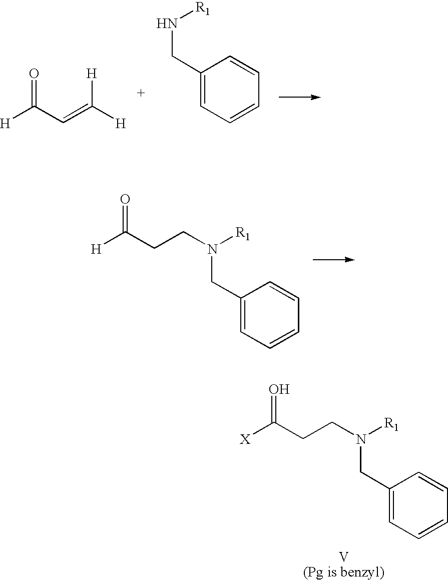

Alternatively, a compound of formula V may be prepared by displacement of a suitable leaving group (Lg) from a compound of formula VI. More specially, a compound of formula VI where Lg is tosyl is reacted with a protected amine in the presence of a suitable base such as potassium carbonate to provide a compound of formula V. A compound of formula VI where Lg is tosyl may be prepared by reacting a 1,3-diol with tosyl chloride in the presence of a suitable base such as triethylamine.

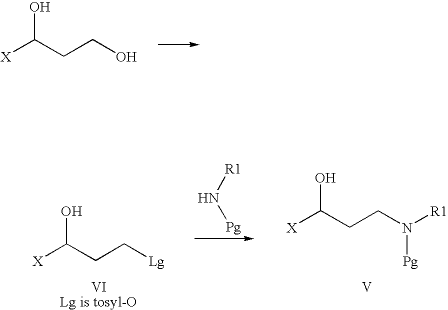

The preparation of 1,3-diols is well known to the skilled artisan. For example, 1,3-diols may be prepared in enantiomeric form by reducing a compound of formula VII or VIII with sodium bis(2-methoxyethoxy)aluminum hydride or lithium aluminum hydride respectively. A compound of formula VII may be prepared by treating an allylic alcohol with asymmetric epoxidation reagents (Synthesis, 1986, 2, p. 89). A compound of formula VIII may be prepared by reacting a β-ketoester with enantioselective reducing agents.

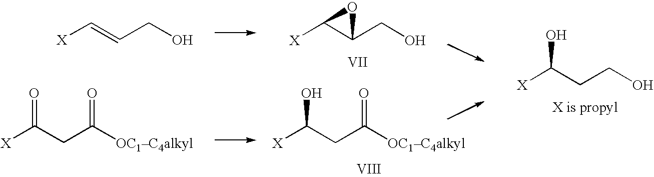

Alternatively, a compound of formula VIII may be prepared in racemic form by reacting an aldehyde with an alkyl actetate pretreated with the appropriate base all in a suitable solvent. Appropriate bases include lithium diisopropylamide and suitable solvents include THF.

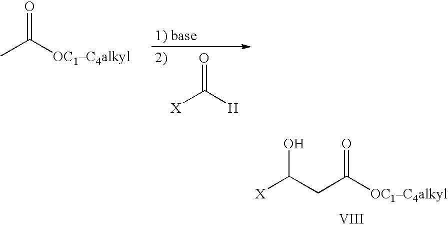

The preparation of phenols and naphthols corresponding to Y—OH are well know to the skilled artisan. Example methods include the reaction of anilines and naphthyl amines (Y—NH₂) with diazotization reagents such as NaNO₂, urea and sulfuric acid under hydrolytic conditions to give the corresponding phenols and naphthols directly. Alternatively, anilines and naphthyl amines may be reacted with boron trifluoride etherate and t-butyl nitrite in a suitable solvent such as ether to give the corresponding diazonium tetrafluoroborate salts in isolated form. The diazonium tetrafluoroborate salts may be reacted with metal oxide salts such as copper (I) oxide and copper (II) nitrate in water to give the corresponding phenols and naphthols.

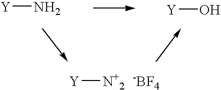

The corresponding ethers and sulfides can generally be prepared as follows:

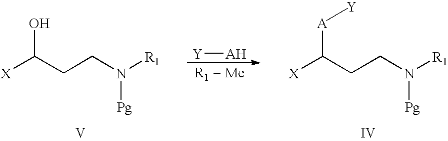

The propanols can be subjected to O-arylation or S-arylation reactions. Various O-arylation conditions can be used such as the Mitsunobu reaction, wherein roughly equal quantities of the heteroaryl alcohol and the 1-X,3-aminopropanol are stirred at temperatures of between 0° C. and reflux in a polar non protic solvent such as toluene, with a complexing agent such as 1,1′-(azodicarbonyl)dipiperidine, or another derivative, and a phosphine ligand such as tributylphosphine. This type of reaction is well known and further combinations of the Mitsunobu reagents can be found in Organic Preparations and Procedures Int., 1996, 28, 2, 165 and references therein. For converting hydroxy to aryl sulfide it is preferred to react the propanol species with Y—SH, (cyanomethyl)trimethylphosphonium iodide (Tetrahedron, 2001, 57, 5451-5454) and diisopropylamine in propionitrile.

Alternatively, a compound of formula IV may be prepared by nucleophilic aromatic substitution. For instance, a compound of formula V is reacted with Y-Lg and a base in a suitable solvent to provide a compound of formula I where A is O. The group Lg is an appropriate leaving group such as fluoro. Appropriate bases include sodium hydride and suitable solvents include DMSO.

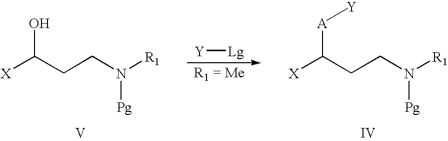

The corresponding protected amines can be readily converted to the amines corresponding to a compound of formula I by standard methods. For example, trifluoroacetic acid can be used for deprotection of a compound of formula IV where Pg is N-t-butyloxycarbonyl and 1-chloroethyl chloroformate in dichloroethane followed by methanol can be used to deprotect a compound of formula I where Pg is benzyl. Finally, resolution of the obtained racemates can be obtained by procedures know to the skilled artisan such as chiral chromatography.

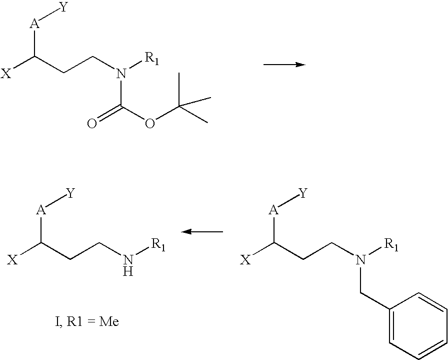

Compounds of the present invention are inhibitors, preferably selective inhibitors, of the reuptake of both serotonin and norepinephrine and as such are useful as pharmaceuticals. They are particularly useful for the treatment of pain.

For clinical purposes, pain may be divided into two categories: acute pain and persistent pain. Acute pain is provoked by noxious stimulation produced by injury and/or disease of skin, deep somatic structures or viscera, or abnormal function of muscle or viscera that does not produce actual tissue damage. On the other hand, persistent pain can be defined as pain that persists beyond the usual course of an acute disease or a reasonable time for an injury to heal or that is associated with a chronic pathologic process that causes continuous pain or the pain recurs at intervals for months or years. If pain is still present after a cure should have been achieved, it is considered persistent pain. For the purpose of the present invention, persistent pain can be chronic non-remitting or recurrent. The difference in definition between acute and persistent pain is not merely semantic but has an important clinical relevance. For example, a simple fracture of the wrist usually remains painful for a week to 10 days. If the pain is still present beyond the typical course of treatment, it is likely that the patient is developing reflex sympathetic dystrophy, a persistent pain syndrome that requires immediate effective therapy. Early and effective intervention potentially prevents the undue disability and suffering, and avoids the potential development of a condition that becomes refractory to therapy.

Acute and chronic pain differ in etiology, mechanisms, pathophysiology, symptomatology, diagnosis, therapy, and physiological responses. In contrast to the transitory nature of acute pain, persistent pain is caused by chronic pathologic processes in somatic structures or viscera, by prolonged and sometimes permanent dysfunction of the peripheral or central nervous system, or both. Also, persistent pain can sometimes be attributed to psychological mechanisms and/or environmental factors.

Current therapies for persistent pain include opiates, barbiturate-like drugs such as thiopental sodium and surgical procedures such as neurectomy, rhizotomy, cordotomy, and cordectomy.

The compounds of the present invention are indicated in the treatment of persistant pain and references herein to methods of using the instant invention for the treatment or prevention of pain are intended to include persistent pain.

Further, the present invention provides a compound of formula I or a pharmaceutically acceptable salt thereof, for use as a pharmaceutical; and a compound of formula I or a pharmaceutically acceptable salt thereof, for use as a selective inhibitor of the reuptake of both serotonin and norepinephrine.

The present compounds and salts may be indicated in the treatment of disorders associated with serotonin and norepinephrine dysfunction in mammals. Preferred mammals are humans.

The term “serotonin and norepinephrine dysfunction” as used herein refers to a reduction in the amount of serotonin and norepinephrine neurotransmitters within the synaptic cleft below that which would be considered to be normal or desirable for a species, or an individual within that species. Thus the phrase “disorders associated with serotonin and norepinephrine dysfunction in mammals” refers to disorders which are associated with a reduction in the amount of serotonin and norepinephrine neurotransmitters within the synaptic cleft below that which would be considered to be normal or desirable for the mammalian species, or an individual within the species, in question. Some examples of disorders currently believed to be associated with reduced levels of serotonin and norepinephrine within the synaptic cleft include depression, OCD, anxiety, memory loss, urinary incontinence (including stress urinary incontinence and urge incontinence), conduct disorders, attention-deficit disorder (including ADHD), obesity, hot flushes/flashes, pain (including inflammatory pain, neuropathic pain, non-neuropathic non-inflammatory pain, persistent pain, persistent pain of inflammatory and/or neuropathic origin, headache and migraine), eating disorders (including bulimia and anorexia nervosa), inflammatory bowel disorders, functional bowel disorders, dyspepsia, Crohn's disease, iletis, ischemic bowel disease, ulcerative colitis, gastroesophageal reflux for functional bowel disorders, irritable bowel syndrome, insterstitial cystitis, urethral syndrome, gastric motility disorders, substance abuse (including alcoholism, tobacco abuse, smoking cessation, symptoms caused by withdrawal or partial withdrawal from the use of tobacco or nicotine and drug addiction including cocaine abuse), dementia of ageing, senile dementia, Alzheimer's, Parkinsonism, social phobia, disruptive behavior disorders, impulsive control disorders, borderline personality disorder, chronic fatigue syndrome, panic disorders, post-traumatic stress disorder, schizophrenia, gastrointestinal disorders, cardiovascular disorders, emesis, sleep disorders, cognitive disorders, psychotic disorders, brain trauma, premenstrual syndrome or late luteal syndrome, sexual dysfunction (including premature ejaculation and erectile difficulty), autism, mutism and trichotilomania. The compounds of the present invention are particularly suitable for the treatment of pain.

The compounds of the present invention are also indicated for the treatment of disorders which are ameliorated by an increase in the amount of serotonin and norepinephrine neurotransmitters within the synaptic cleft of a mammal above that which would be considered to be normal or desirable for the mammalian species, or an individual within the species, in question.

The term “treatment” as used herein refers to both curative and prophylactic treatment of disorders associated with norepinephrine dysfunction.

The present invention also provides the use of a compound of formula I, or a pharmaceutically acceptable salt thereof, in the manufacture of a medicament for selectively inhibiting the reuptake of serotonin and norepinephrine; the use of a compound of formula I, or a pharmaceutically acceptable salt thereof, in the manufacture of a medicament for the treatment of disorders associated with serotonin and norepinephrine dysfunction in mammals; the use of a compound of formula I, or a pharmaceutically acceptable salt thereof, in the manufacture of a medicament for the treatment of a disorder selected from those listed above and in particular selected from depression, OCD, anxiety, memory loss, urinary incontinence, conduct disorders, ADHD, obesity, alcoholism, smoking cessation, hot flushes/flashes and pain; and the use of a compound of formula I, or a pharmaceutically acceptable salt thereof, in the manufacture of a medicament for the treatment of a disorder selected from depression, urinary incontinence, particularly stress induced urinary incontinence, and more especially, pain. The present invention further provides a compound of formula I for treating disorders associated with serotonin and norepinephrine dysfunction in mammals, for example a disorder selected from those listed above and in particular selected from depression, OCD, anxiety, memory loss, urinary incontinence, conduct disorders, ADHD, obesity, alcoholism, smoking cessation, hot flushes/flashes and pain, especially depression, urinary incontinence, particularly stress induced urinary incontinence, and, more especially, pain.

Further the present invention provides a method for selectively inhibiting the reuptake of serotonin and norepinephrine in mammals, comprising administering to a patient in need thereof an effective amount of a compound of formula I or a pharmaceutically acceptable salt thereof; a method for treating disorders associated with serotonin and norepinephrine dysfunction in mammals, comprising administering to a patient in need thereof an effective amount of a compound of formula I or a pharmaceutically acceptable salt thereof; and a method for treating a disorder selected from those listed above and in particular selected from depression, OCD, anxiety, memory loss, urinary incontinence, conduct disorders, ADHD, obesity, alcoholism, smoking cessation, hot flushes/flashes and pain, comprising administering to a patient in need thereof an effective amount of a compound of formula I or a pharmaceutically acceptable salt thereof. “Patient” includes both human and other mammals. “Effective amount” means an amount of a compound/composition according to the present invention effective in producing the desired therapeutic effect.

The present invention includes the pharmaceutically acceptable salts of the compounds of formula I. Suitable salts include acid addition salts, including salts formed with inorganic acids, for example hydrochloric, hydrobromic, nitric, sulphuric or phosphoric acids, or with organic acids, such as organic carboxylic acids, for example acedic, pyruvic, lactobionic, glycolic, oxalic, maleic, hydroxymaleic, fumaric, malic, succinic, trifluoroacedic, tartaric, citric, salicylic, o-acetoxybenzoic acids, or organic sulphonic, 2-hydroxyethane sulphonic, toluene-p-sulphonic, bisethanesulphonic acid or methanesulphonic acid. Preferred salts include hydrochloric and succinic acid addition salts.

In addition to the pharmaceutically acceptable salts, other salts are included in the invention. They may serve as intermediates in the purification of compounds or in the preparation of other, for example pharmaceutically acceptable, acid addition salts, or are useful for identification, characterization or purification.

While all the compounds of the present invention are believed to inhibit the reuptake of serotonin and norepinephrine in mammals there are certain of these compounds which are preferred for such uses. Preferred values for A, X, Y, R₁ and R₂ and substituents for each have been set out above.

### Preparations

### Preparation 1

### (2R,3R)-(3-Propyl-oxiranyl)-methanol

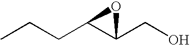

Add trans-2-hexen-1-ol (12 mL, 102 mmol), (−)-diethyl tartrate (2.1 mL, 12.3 mmol), and Ti(OⁱPr)₄ (3.0 mL, 10.2 mmol) to a cooled (−20° C.) solution of activated, dried, crushed 4 Å molecular sieves (50 g) in dichloromethane (700 mL). After 30 minutes, add a dry [JACS, 1987, ]109, 5765] solution of t-BuOOH in dichloromethane (˜5M in dichloromethane, 57 mL, 285 mmol). Stir for 4 hours at −20° C. and filter the solids. Add 700 mL of 15% L-tartartic acid to the filtrate and stir for 20 minutes. Separate the layers, extract the aqueous layer with dichloromethane, and concentrate the combined organic extracts in vacuo. Add 300 mL diethyl ether to residue and cool to 0° C. Add cool (0° C.) 15% NaOH, stir for 15 minutes, separate, and extract aqueous layer with diethyl ether. Wash the organic layer with aqueous saturated sodium chloride, dry over anhydrous MgSO₄, filter, and concentrate in vacuo. Purify on silica gel eluting with 0-50% EtOAc/hexanes to give (2R,3R)-(3-propyl-oxiranyl)-methanol (5.69 g, 48%). 1H NMR (CDCl₃) δ 3.97-3.88 (m, 1H), 3.68-3.59 (m, 1H), 3.00-2.91 (m, 2H), 1.83-1.68 (m, 1H), 1.61-1.41 (m, 4H), 0.97 (t, 3H).

A method similar to that described in Preparation 1 is used to prepare the following compound:

| Preparation | Name | ¹H NMR |
|---|---|---|
| 2 | (2S,3S)-(3-Propyl- | (CDCl₃) δ 3.97-3.88(m, 1H), |
| | oxiranyl)-methanol | 3.68-3.59(m, 1H), 3.00-2.91(m, 2H), |
| | | 1.83-1.68(m, 1H), 1.61-1.41(m, 4H), |
| | | 0.97(t, 3H). |

### Preparation 3

### (R)-Hexane-1,3-diol

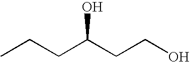

Add sodium bis(2-methoxyethoxy) aluminum hydride (65% wt in toluene, 7.0 mL, 22.4 mmol) to a cooled (0° C.) solution of (R,R)-(3-propyl-oxiranyl)-methanol (0.999 g, 8.60 mmol) in anhydrous THF (48 mL) and stir at 0° C. overnight, and then add 1N HCl at 0° C. Filter the solids, then separate the layers from the filtrate. Extract the aqueous layer with dichloromethane. Boil the solids in EtOAc, decant the solvent, and dry the combined organic extracts over anhydrous MgSO₄, filter, and concentrate in vacuo. Purify on silica gel eluting with 100% EtOAc to give (R)-hexane-1,3-diol (550 mg, 54%). ¹H NMR (CDCl₃) δ 3.93-3.79 (m, 3H), 2.62 (dd, 1H), 2.55 (d, 1H), 1.75-1.58 (m, 2H), 1.54-1.28 (m, 4H), 0.96-0.89 (m, 3H).

### Preparation 4

### (R)-3-Hydroxy-hexanoic acid ethyl ester

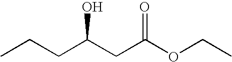 Catalyst (1):

Combine dichloro(1,5-cyclooctadiene)ruthenium (117 mg, 0.417 mmol), (R)-BINAP (300 mg, 0.482 mmol), and NEt₃ (82.5 μL, 0.59 mmol) in anhydrous toluene (13.5 mL) under N₂. Heat the reaction mixture at 140° for 4 hours, and then cool to ambient temperature. Add THF to the resulting red jell, until a solution results. Concentrate the reaction mixture in vacuo, and add THF (30 mL) to the give the catalyst (1) in solution. This solution is used directly for the hydrogenation.

### (R)-3-Hydroxy-hexanoic acid ethyl ester

In a Fisher Porter tube, add the above catalyst (1) solution (10 mL) to a solution of ethyl butyrylacetate (25 g, 0.158 mol) in methanol (100 mL) under N₂. Pressurized with H2 (50 psig) and heat at 80° C. for 5 hours. Concentrate the reaction mixture in vacuo. Purify the residue by vacuum distillation (24.45 g, 96%, 97.4% ee). Chiral GC (30 m×0.25 mm×0.25 Am, BETA-Dex 225, 100° C.) second eluting isomer, (18.93 minutes).

### Preparation 5

### (S)-3-Hydroxy-hexanoic acid ethyl ester

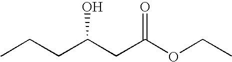 Catalyst (2):

Combine dichloro(1,5-cyclooctadiene)ruthenium (117 mg, 0.417 mmol), (S)-BINAP (300 mg, 0.482 mmol), and NEt₃ (82.5 μL, 0.59 mmol) in anhydrous toluene (13.5 mL) under N₂. Heat the reaction mixture at 140° for 4 hours, and then cool to ambient temperature. Add THF to the resulting red jell, until a solution results. Concentrate the reaction mixture in vacuo, and add THF (30 mL) to the give the catalyst (1) in solution. This solution is used directly for the hydrogenation.

### (S)-3-Hydroxy-hexanoic acid ethyl ester

In a Fisher Porter tube, add the above catalyst (2) solution (10 mL) to a solution of ethyl butyrylacetate (25 g, 0.158 mol) in methanol (100 mL) under N₂. Pressurize with H₂ (50 psig) and heat at 80° C. for 5 hours. Concentrate the reaction mixture in vacuo. Purify the residue by vacuum distillation (25.0 g, 100%, 97.4% ee). Chiral GC (30 m x 0.25 mm×0.25 μm, BETA-Dex 225, 100° C.) first eluting isomer, (18.72 minutes).

### Preparation 6

### Hexane-1,3-diol

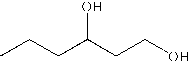

Add a solution of 3-hydroxy-hexanoic acid ethyl ester (20.0 g, 124.8 mmol) in anhydrous THF (20 mL) to a cooled (0° C.) solution of lithium aluminum hydride (10.00 g, 263.5 mmol) in anhydrous THF (500 mL). After 10 minutes at 0° C., warm the reaction mixture to ambient temperature. After 1.5 hours, cool to 0° C. and add 10 mL water, 10 mL 15% NaOH, and 30 mL water. Stir 15 minutes, filter the solids, and wash the solids with EtOAc. Combine the filtrate and dry over anhydrous Na₂SO₄, filter, and concentrate in vacuo. Purify the residue on silica gel eluting with 0-100% EtOAc/hexanes to give hexane-1,3-diol (12.95 g, 88%). Mass spectrum (ion spray): m/z=119 (M+1), ¹H NMR(CDCl₃) δ 3.93-3.79 (m, 3H), 2.62 (dd, 1H), 2.55 (d, 1H), 1.75-1.58 (m, 2H), 1.54-1.28 (m, 4H), 0.96-0.89 (m, 3H).

A method similar to that described in Preparation 6 is used to prepare the following compounds:

| | | Mass | |
|---|---|---|---|
| | | Spectrum | |
| | | (ion spray) | |
| | | m/z | |
| Preparation | Name | (M+1) | ¹H NMR |
| 7 | (S)-Hexane- | 119 | (CDCl₃) δ 3.93-3.79(m, 3H), |
| | 1,3-diol | | 2.62(dd, 1H), 2.55(d, 1H), |
| | | | 1.75-1.58(m, 2H), 1.54-1.28(m, |
| | | | 4H), 0.96-0.89(m, 3H) |
| 8 | (R)-Hexane- | 119 | (CDCl₃) δ 3.93-3.79(m, 3H), |
| | 1,3-diol | | 2.62(dd, 1H), 2.55(d, 1H), |
| | | | 1.75-1.58(m, 2H), 1.54-1.28(m, |
| | | | 4H), 0.96-0.89(m, 3H) |

### Preparation 9

### 3-Hydroxy-4-methyl-pentanoic acid tert-butyl ester

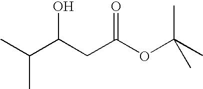

Add lithium diisopropylamine (2M in heptane/tetrahydrofuran/ethylbenzene, 208 mL, 416 mmol) dropwise to a solution of t-butyl acetate (64.4 g, 555 mmol) in THF (350 mL) at −78° C. After 2 hours at −78° C., add isobutyraldehyde (10.0 g, 139 mmol) as a solution in 50 mL THF and stir for 3-5 hours before adding water. Separate the layers and extract the aqueous layer with diethyl ether then EtOAc. Combine the organic extracts, dry with anhydrous MgSO₄, filter and concentrate in vacuo. Purify the residue on silica gel eluting with 10% EtOAc/hexanes to give 3-hydroxy-4-methyl-pentanoic acid tert-butyl ester (20.4 g, 78%). ¹H NMR (CDCl₃) δ 3.76-3.69 (m, 1H), 3.06 (brs, 1H), 2.44-2.28 (m, 2H), 1.73-1.64 (m, 1H), 1.45 (s, 9H), 0.93 (dd, 6H).

A method similar to that described in Preparation 9 is used to prepare the following compound:

| Preparation | Name | ¹H NMR |
|---|---|---|
| 10 | 3-Hydroxy-5,5-dimethyl- | (CDCl₃) δ 4.16-4.10(m, 1H), |
| | hexanoic acid tert-butyl | 2.36(d, 2H), 2.04(s, 1H), |
| | ester | 1.49-1.45(m, 11H), 0.96(s, 9H). |

### Preparation 11

### 4-Cyclopropyl-3-hydroxy-butyric acid tert-butyl ester

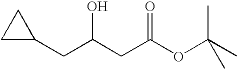 Reaction A and Reaction B are Run in Tandem:

Reaction A: Dissolve oxalyl chloride (9.3 mL, 106.7 mmol) in dichloromethane (140 mL) at ambient temperature, then cool to −78° C. and add slowly a solution of DMSO (11.22 g, 143.6 mmol) in dichloromethane (20 mL) while venting the mixture and stir the reaction at −78° C. for 20 minutes. Add 2-cyclopropyl-ethanol (6.11 g, 71.21 mmol) in dichloromethane (20 mL). Warm to 0° C. and add dichloromethane (25 mL) to assist with stirring and mix for 30 minutes. Dilute this reaction mixture with 100 mL THF and pour into a cold (−78° C.) solution of Reaction B.

Reaction B: Add a solution of tert-butyl acetate (62 mL, 424 mmol) in THF (60 mL) to a cold solution (−78° C.) of lithium diisopropylamine (2M in heptanes/tetrahydrofuran/ethylbenzene, 180 mL, 360 mmol) in anhydrous THF (700 mL) and stir at −78° C. for 1.5-2 hours. Add crude Reaction A prepared as described above and rinse with 100 mL anhydrous THF. Stir at −78° C. for 1.5 hours then add water/diethyl ether and warm to ambient temperature overnight. Separate the layers and extract the aqueous layer with diethyl ether (3×). Dry the combined organic extracts over anhydrous MgSO₄, filter, and concentrate in vacuo. Purify the reaction on silica gel eluting with 20% EtOAc/hexanes to give 4-cyclopropyl-3-hydroxy-butyric acid ter-tbutyl ester (10.65 g. 75%). ¹H NMR (CDCl₃) δ 4.11-4.05 (m, 1H), 3.12 (d, 1H), 2.53 (dd, 1H), 2.38 (dd, 1H), 1.60-1.48 (m, 1H), 1.47 (s, 9H), 1.32-1.23 (m, 1H), 0.80-0.73 (m, 1H), 0.51-0.46 (m, 2H), 0.13-0.04 (m, 2H).

### Preparation 12

### 4-Methyl-pentane-1,3-diol

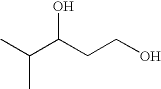

Add lithium aluminum hydride (1.0 M in tetrahydrofuran, 64 mL, 64 mmol) dropwise to a cool (0° C.) solution of 3-hydroxy-4-methyl-pentanoic acid tert-butyl ester (8.0 g, 43 mmol) in anhydrous THF (500 mL). After 2 hours, carefully add 2 mL water, 6 mL 15% NaOH, and 6 mL water. Filter and wash the solids with EtOAc. Combine the filtrates, dry with anhydrous Na₂SO₄, filter, and concentrate in vacuo. Purify the residue on silica gel eluting with 50% EtOAc/hexanes) to give 4-methyl-pentane-1,3-diol (3.1 g, 62%). ¹H NMR (CDCl₃) δ 3.92-3.78 (m, 2H), 3.63-3.58 (m, 1H), 2.66 (brs, 2H), 1.71-1.61 (m, 3H), 0.91 (dd, 6H).

A method similar to that described in Preparation 12 is used to prepare the following compounds:

| Preparation | Name | ¹H NMR |
|---|---|---|
| 13 | 5,5-Dimethylhexane-1,3-diol | (CDCl₃) δ 4.05-3.97(m, 1H), 3.87-3.76(m, |
| | | 2H), 3.01(s, 2H), 1.68-1.63(m, 2H), |
| | | 1.49-1.41(m, 1H), 1.37-1.31(m, 1H), 0.95(s, 9H) |
| 14 | 4-Cyclopropyl-butane-1,3-diol | (CDCl₃) δ 4.03-3.93(m, 1H), 3.92-3.80(m, |
| | | 2H), 2.60(s, 2H), 1.84-1.66(m, 2H), |
| | | 1.47-1.38(m, 2H), 0.78-0.68(m, 1H), |
| | | 0.54-0.43(m, 2H), 0.17-0.04(m, 2H) |

### Preparation 15

### 6,6,6-Trifluoro-hexane-1,3-diol

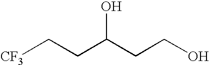

Add lithium diisopropyl amine (2M in heptane/THF/ethylbenzene, 29 mL, 58 mmol) dropwise to a solution of tert-butyl acetate (12.5 mL, 92.8 mmol) in THF (100 mL) at −78° C. After 1.25 hours, add 4,4,4-trifluoro-butyraldehyde (1.47 g, 11.66 mmol) in 10 mL THF. After 1.75 hours, add water/EtOAc and warm to ambient temperature. Separate the layers and extract the aqueous layer with EtOAc. Dry the combined organic extracts over anhydrous MgSO₄, filter, and concentrate in vacuo. Then add anhydrous dichloromethane (100 mL) to the crude residue and cool to 0° C. Add DIBAL-H (1M in dichlormethane, 60 mL, 60 mmol) dropwise and stir at 0° C. for 1.5 hours. Carefully pour the reaction mixture into aqueous saturated sodium potassium tartrate and stir for 3 days. Separate and extract the aqueous layer with EtOAc (2×) and diethyl ether (2×). Dry the combined organic extracts over anhydrous Na₂SO₄, filter, and concentrate in vacuo. Purify the residue on silica gel eluting with 50% EtOAc/dichloromethane to give 6,6,6-trifluoro-hexane-1,3-diol (0.654 g, 33%). 1H NMR (CDCl₃) δ 4.00-3.83 (m, 3H), 2.87-2.80 (m, 1H), 2.41-2.08 (m, 2H), 2.05-1.92 (m, 1H), 1.77-1.67 (m, 4H).

### Preparation 16

### Toluene-4-sulfonic acid 3-hydroxy-hexyl ester

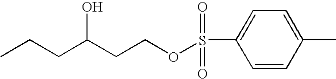

Add pyridine (8.9 mL, 110 mmol) and triethylamine (15.2 mL, 109 mmol) to a cooled (0° C.) solution of hexane-1,3-diol (12.92 g, 8.3 mmol) in anhydrous dichloromethane (10 mL). Add p-toluene sulfonyl chloride (1.9 g, 109 mmol) slowly in portions. Stir at 0° C. for 1 hour, at ambient temperature for 1 hour, and then add 5N HCl. Separate the layers, and extract the aqueous layer with EtOAc. Wash the organic extracts with 1N HCl and aqueous saturated sodium chloride, dry over anhydrous Na₂SO₄, filter, and concentrate in vacuo. Purify on silica gel eluting with 5% EtOAc/hexanes to give toluene-4-sulfonic acid 3-hydroxy-hexyl ester (23.40 g, 79%). Mass spectrum (ion spray): m/z=273 (+1), ¹H NMR (CDCl₃) δ 7.78 (d, 2H), 7.34 (d, 2H), 4.30-4.22 (m, 1), 4.16-4.09 (m, 1H), 3.77-3.69 (m, 1H), 2.44 (s, 3H), 1.88-1.80 (m, 1H), 1.68-1.60 (m, 2H), 1.46-1.27 (m, 4H), 0.94-0.88 (m, 3H).

A method similar to that described in Preparation 16 is used to prepare the following compounds:

| Preparation | Name | ¹H NMR |
|---|---|---|
| 17 | (R)-Toluene-4-sulfonic acid | (CDCl₃) δ 7.78(d, 2H), 7.34(d, 2H), |
| | 3-hydroxy-hexyl ester | 4.30-4.22(m, 1H), 4.16-4.09(m, 1H), 3.77-3.69(m, 1H), |
| | | 2.46(s, 3H), 1.89-1.80(m, 1H), 1.69-1.60(m, |
| | | 1H), 1.46-1.27(m, 4H), 0.94-0.88(m, 3H) |
| 18 | (S)-Toluene-4-sulfonic acid 3- | (CDCl₃) δ 7.78(d, 2H), 7.34(d, 2H), |
| | hydroxy-hexyl ester | 4.30-4.22(m, 1H), 4.16-4.09(m, 1H), 3.77-3.69(m, 1H), |
| | | 2.46(s, 3H), 1.89-1.80(m, 1H), 1.69-1.60(m, |
| | | 1H), 1.46-1.27(m, 4H), 0.94-0.88(m, 3H) |
| 19 | Toluene-sulfonic acid 3- | (CDCl₃) δ 7.79(d, 2H), 7.34(d, 2H), |
| | hydroxy-4-methyl-pentyl | 4.30-4.23(m, 1H), 4.18-4.08(m, 1H), 3.51-3.46(m, 1H), |
| | ester | 2.45(s, 3H), 1.89-1.82(m, 1H), 1.67-1.57(m, |
| | | 3H), 0.89(d, 6H) |
| 20 | Toluene-4-sulfonic acid 3- | (CDCl₃) δ 7.79(d, 2H), 7.34(d, 2H), |
| | hydroxy-5,5-dimethyl-hexyl | 4.30-4.22(m, 1H), 4.15-4.08(m, 1H), 2.45(s, 3H), |
| | ester | 1.84-1.75(m, 1H), 1.71-1.63(m, 1H), 1.40-1.24(m, |
| | | 2H), 0.94(s, 9H) |
| 21 | Toluene-4-sulfonic acid 6,6,6- | (CDCl₃) δ 7.80(d, 2H), 7.36(d, 2H), |
| | trifluoro-3-hydroxy-hexyl | 4.34-4.27(m, 1H), 4.14-4.08(m, 1H), 3.86-3.81(brm, 1H), |
| | ester | 2.47(s, 3H), 2.37-2.26(m, 1H), 2.19-2.09(m, |
| | | 1H), 1.95-1.84(m, 2H), 1.74-1.60(m, 3H) |
| 22 | Toluene-4-sulfonic acid 4- | (CDCl₃) δ 7.80(d, 2H), 7.35(d, 2H), |
| | cyclopropyl-3-hydroxy-butyl | 4.30-4.23(m, 1H), 4.19-4.11(m, 1H), 3.88-3.81(brm, 1H), |
| | ester | 2.46(s, 3H), 1.96-1.88(m, 1H), 1.80(brs, 1H), |
| | | 1.74-1.65(m, 1H), 1.40-1.31(m, 2H), |
| | | 0.75-0.67(m, 1H), 0.53-0.42(m, 2H), 0.14-0.01(m, 2H) |

### Preparation 23

### (R)-1-(Benzyl-methyl-amino)-hexan-3-ol

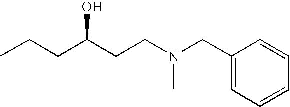

Add K₂CO₃ (12.89 g, 93.26 mmol), NaI (1.80 g, 12.01 mmol), and N-methylbenzyl amine (6.3 mL, 48.82 mmol) to a solution of (R)-toluene-4-sulfonic acid 3-hydroxy-hexyl ester (12.68 g, 46.56 mmol) in anhydrous acetonitrile (200 mL). Heat and stir the reaction mixture at reflux for 2-4 hours. Cool the mixture to ambient temperature and filter through Celite®. Wash solids with EtOAc, and concentrate the combined filtrates in vacuo. Purify the residue on silica gel eluting with 0.3% NH₄OH/3% ethanol/chloroform to give (R)-1-(benzyl-methyl-amino)-hexan-3-ol (8.00 g, 78%). Mass spectrum (ion spray): m/z=222 (M+1), ¹H NMR (CDCl₃) δ 7.34-7.22 (m, 5H), 6.14 (brs, 1H), 3.79-3.72 (m, 1H), 3.63 (d, 1H), 3.42 (d, 1H), 2.80-2.72 (m, 1H), 2.56-2.52 (m, 1H), 2.21 (s, 3H), 1.72-1.61 (m, 1H), 1.57-1.29 (in, 5H), 0.93 (t, 3H).

A method similar to that described in Preparation 23 is used to prepare the following compounds:

| | | Mass | |
|---|---|---|---|
| | | Spectrum | |
| | | (ion spray) | |
| Preparation | Name | m/z (M+1) | ¹H NMR |
| 25 | (S)-1-(Benzyl-methyl-amino)- | 222 | (CDCl₃) δ 7.35-7.23(m, 5H), |
| | hexan-3-ol | | 6.13(s, 1H), 3.78-3.72(m, 1H), |
| | | | 3.62(d, 1H), 3.41(d, 1H), |
| | | | 2.78-272(m, 1H), 2.57-2.52(m, 1H), |
| | | | 2.20(s, 3H), 1.70-1.60(m, 1H), |
| | | | 1.55-1.29(m, 5H), 0.93-0.88(m, |
| | | | 3H) |
| 26 | 1-(Benzyl-methyl-amino)- | NA | (CDCl₃) δ 7.36-7.25(m, 5H), |
| | 6,6,6-trifluoro-hexan-3-ol | | 6.43(brs, 1H), 3.81-3.73(m, 1H), |
| | | | 3.53(dd, 2H), 2.82-2.73(m, 1H), |
| | | | 2.61-2.55(m, 1H), 2.41-2.28(m, |
| | | | 1H), 2.23(s, 3H), 2.19-2.08(m, |
| | | | 1H), 1.73-1.61(m, 3H), |
| | | | 1.55-1.50(m, 1H) |
| 27 | 1-(Benzyl-methyl-amino)-4- | 222 | |
| | methyl-3-pentanol | | |
| 28 | 1-(Benzyl-methyl-amino)- | 250 | (CD₃OD) δ 7.32-7.23(m, 5H), |
| | 5,5-dimethyl-hexan-3-ol | | 3.82-3.76(m, 1H), 3.52(dd, 2H), |
| | | | 2.69-2.61(m, 1H), 2.56-2.47(m, |
| | | | 1H), 2.20(s, 3H), 1.72-1.57(m, |
| | | | 2H), 1.45-1.25(m, 2H), 0.95(s, |
| | | | 9H) |
| 29 | 4-(Benzyl-methyl-amino)-1- | 234 | (CD₃OD) δ 7.33-7.23(m, 5H), |
| | cyclopropyl-butan-2-ol | | 3.77-3.70(m, 1H), 3.53(dd, 2H), |
| | | | 2.73-2.65(m, 1H), 2.56-2.50(m, |
| | | | 1H), 2.21(s, 3H), 1.78-1.61(m, |
| | | | 2H), 1.52-1.44(m, 1H), |
| | | | 1.23-1.15(m, 1H), 0.81-0.73(m, 1H), |
| | | | 0.48-0.41(m, 2H), 0.09-0.01(m, 2H) |

### Preparation 30

### 3-Bromo-N-methoxy-N-methyl-propionamide

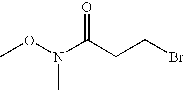

Add pyridine (28.0 mL, 346.2 mmol) to a cooled (0° C.) solution of 3-bromopropionyl chloride (26.37g, 15.38 mmol) and N,O-dimethylhydroxyl amine hydrochloride (15.78 g, 161.8 mmol) in anhydrous dichloromethane (250 mL). Warm the reaction mixture to ambient temperature and stir for 3 days. Remove the solvents in vacuo and add aqueous saturated sodium chloride solution and extract with diethyl ether/dichloromethane (1:1). Dry the combined organics over anhydrous Na₂SO₄, filter, and concentrate in vacuo. Purify on silica gel eluting with 17% acetone/hexanes to give 3-bromo-N-methoxy-N-methyl-propionamide (23.42, 78%). Mass spectrum (ion spray): m/z=198 (M+1), ¹H NMR (CDCl₃) δ 3.80 (t, 1H), 3.71 (s, 3H), 3.63 (t, 1H), 3.19 (s, 3H), 3.03 (brt, 1H), 2.91 (brt, 1H).

### Preparation 31

### 3-(Benzyl-methyl-amino)-N-methoxy-N-methyl-propionamide

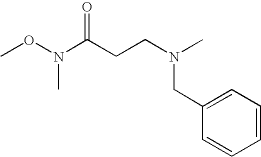

Add K₂CO₃ (62.6 g, 452.9 mmol) and N-benzylmethyl amine (15.5 mL, 120.1 mL) to a solution of 3-bromo-N-methoxy-N-methyl-propionamide (23.42 g, 119.5 mmol) in anhydrous acetonitlile (500 mL). Heat the reaction mixture at reflux for 2.5 hours, cool to ambient temperature, filter, rinse the solids with acetonitrile, and concentrate the filtrate in vacuo. Purify on silica gel eluting 50% acetone/hexanes. Collect impure fractions and re-purify on silica gel eluting with 0.5% NH₄OH/5% ethanol/chlorofonn. Repeat purification on impure fractions eluting with 0.45% NH₄OH/4.5% ethanol/chloroform. Combine all clean fractions and concentrate in vacuo to give 3-(benzyl-methyl-amino)-N-methoxy-N-mrethyl-propionamide (21.44 g, 76%). Mass spectrum (ion spray): m/z=237 (M+1), ¹H NMR (CDCl₃) δ 7.34-7.21 (m, 5H), 3.65 (s, 3H), 3.53 (s, 2H), 3.17 (s, 3H), 2.80-2.74 (m, 2H), 2.67-2.62 (m, 2H), 2.22 (s, 3H).

### Preparation 32

### 1-(Benzyl-methyl-amino)-6-methyl-heptan-3-one

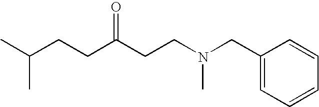

Add 1-bromo-3-methyl-butane (3.05 mL, 25.46 mmol) to a solution of magnesium (0.630 g, 25.92 mmol) in anhydrous THF (30 mL) and heat to reflux. Cool the reaction mixture to ambient temperature and add to a cool (0° C.) solution of 3-(benzyl-methyl-amino)-N-methoxy-N-methyl-propionamide (2.06 g, 87.17 mmol) in THF (30 mL). After 30 minutes at 0° C., add aqueous saturated NaHCO₃ and allow the reaction to warm to ambient temperature. Add aqueous saturated NaHCO₃, separate the layers, and extract the aqueous layer with EtOAc. Wash the combined organic extracts with aqueous saturated sodium chloride solution, dry over anhydrous Na₂SO₄, filter, and concentrate in vacuo. Purify on silica gel eluting with 0.25% NH₄OH/2-5%

EtOAc/chloroform and concentrate the appropriate fractions, then load the obtained residue onto an SCX column. Wash column with methanol and then elute the product with 2H NH₃ in methanol to give 1-(benzyl-methyl-amino)-6-methyl-heptan-3-one (1.43 g, 66%). Mass spectrum (ion spray): m/z=248 (M+1), ¹H NMR (CDCl₃) δ 7.35-7.20 (m, 5H), 3.48 (s, 2H), 2.73-2.66 (m, 2H), 2.63-2.56 (m, 2H), 2.42-2.36 (m, 2H), 2.17 (s, 3H), 1.58-1.40 (m, 3H), 0.87 (d, 6H).

### Preparation 33

### 4-(Benzyl-methyl-amino)-butan-2-one

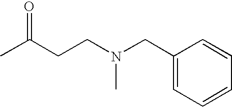

Add methyllithium (1.6 M in diethyl ether, 20.43 mL, 32.7 mmol) dropwise to a solution of 3-(benzyl-methyl-amino)-N-methoxy-N-methyl-propionamide (5.0 g, 21.1 mmol) in THF (100 mL) at −40° C. under N₂ and stir for 1 hour. Add aqueous saturated NH₄Cl (100 mL) at −40° and allow the reaction mixture to warm to ambient temperature. Add aqueous saturated NaHCO₃, separate, and extract the aqueous layer with EtOAc. Wash the combined organic extracts with aqueous saturated sodium chloride solution and dry over anhydrous Na₂SO₄, filter, and concentrate in vacuo. Purify on silica gel eluting with 2.5% ethanol/0.25% NH₄OH/chloroform to give 4-(benzyl-methyl-amino)-butan-2-one (3.85 g, 95%). Mass spectrum (ion spray): m/z=192 (M+1), ¹H NMR (CDCl₃) δ 7.31-7.23 (m, 5H), 3.49 (s, 2H), 2.68 (m, 4H), 2.19 (s, 3H), 2.14 (s, 3H).

### Preparation 34

### 1-(Benzyl-methyl-amino)-pentan-3-one

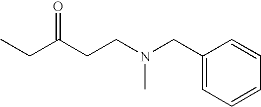

Add ethylmagnesium bromide (3M in diethyl ether, 14.0 mL, 42.20 mmol) dropwise to a solution of 3-(benzyl-methyl-amino)-N-methoxy-N-methyl-propionamide (5.0 g, 21.1 mmol) in THF (100 mL) at −40° C. under N₂ and stir for 2 hours. Add saturated aqueous NH₄Cl at −40° C. and allow the reaction mixture to reach room temperature. Add aqueous saturated NaHCO₃, separate the layers, and extract the aqueous layer with EtOAc. Wash the combined organic extracts with aqueous saturated sodium chloride solution, dry over anhydrous Na₂SO₄, filter, and concentrate in vacuo. Purify on silica gel eluting with 2.5% ethanol/0.25% NH₄OH/chloroform to give 1-(benzyl-methyl-amino)-pentan-3-one (2.6 g, 60%). Mass spectrum (ion spray): m/z=206 (M+1), ¹H NMR (CDCl₃) δ 7.31-7.23 (m, 5H), 3.49 (s, 2H), 2.67 (m, 4H), 2.59-2.40 (m, 2H), 2.19 (s, 3H), 1.05 (t, 3H).

### Preparation 35

### 4-(Benzyl-methyl-amino)-butan-2-ol

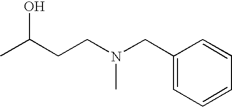

Add NaBH₄ (2.51 g, 66.0 mmol) to a solution of 4-(benzyl-methyl-amino)-butan-2-one (3.85 g, 20 mmol) in methanol (90 mL) at 0° C. under N₂ and stir the reaction mixture for 1 hour at 0° C. before adding water at 0° C. Remove the solvents in vacuo, dissolve the residue in EtOAc, wash the organics layer with water and aqueous saturated sodium chloride solution, dry over anhydrous Na₂SO₄, filter, and concentrate. Purify on silica gel eluting with 3% ethanol/0.3% NH₄OH/chloroform to give 4-(benzyl-methyl-amino)-butan-2-ol (2.96 g, 76%). Mass spectrum (ion spray): m/z=194 (M+1), ¹H NMR (CDCl₃) δ 7.34-7.23 (m, 5H), 6.15 (brs, 1H), 3.94-2.90 (m, 1H), 3.52 (dd, 2H), 2.80-2.73 (m, 1H), 2.56-2.51 (m, 1H), 2.20 (s, 3H), 1.69-1.62 (m, 1H), 1.54-1.48 (m, 1H), 1.16 (d, 3H).

A method similar to that described in Preparation 35 is used to prepare the following compounds:

| | | Mass Spectrum | |
|---|---|---|---|
| | | (ion spray): | |
| Preparation | Name | m/z (M+1) | ¹H NMR |
| 36 | 1-(Benzyl-methyl- | 208 | (CDCl₃) δ 7.33-7.23(m, 5H), |
| | amino)-pentan-3-ol | | 3.68-3.62(m, 2H), 3.41(d, 1H), |
| | | | 2.80-2.73(m, 1H), 2.58-2.53(m, |
| | | | 1H), 2.20(s, 3H), 1.67-1.39(m, |
| | | | 4H), 0.94(t, 3H) |
| 37 | 1-(Benzyl-methyl- | 250 | (CDCl₃) δ 7.36-7.22(m, 5H), |
| | amino)-6-methyl- | | 6.17(brs, 1H), 3.76-3.66(m, 1H), |
| | heptan-3-ol | | 3.52(dd, 2H), 2.81-2.69(m, 1H), |
| | | | 2.59-2.49(m, 1H), 2.20(s, 3H), |
| | | | 1.71-1.28(m, 6H), 1.22-1.11(m. |
| | | | 1H), 0.88(d, 6H) |

### Preparation 38

### 2-(2-Bromo-ethyl)-oxirane

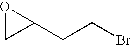

Slowly add a solution of mCPBA (75.0 g, 348 mmol) in anhydrous dichloromethane (750 mL) to a cool (0° C.) solution of 4-bromo-1-butene (30.0 mL, 296 mmol) in anhydrous dichloromethane (175 mL). After addition is complete, warm the reaction mixture to ambient temperature and stir overnight before pouring the reaction mixture into 200 mL 5N NaOH. Separate the layers and wash the organic layer with 5N NaOH, then with water until the water washes are neutral pH. Dry the combined organic extracts over anhydrous MgSO₄, filter, and concentrate the filtrate under reduced pressure (70 mm Hg) to obtain 2-(2-bromo-ethyl)-oxirane (30.81 g, 69%). ¹H NMR (CDCl₃) δ 3.53-3.48 (m, 2H), 3.10-3.05 (m, 1H), 2.82 (t, 1H), 2.56 (dd, 1H), 2.20-1.98 (m, 2H).

### Preparation 39

### Benzyl-methyl-(2-oxiranyl-ethyl)-amine

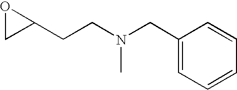

Add K₂CO₃ (46.40 g, 335.7 mmol) and N-methylbenzyl amine (28.0 mL, 217 mmol) to a solution of 2-(2-bromo-ethyl)-oxirane (30.81 g, 204 mmol) in acetone (600 mL) and heat the reaction mixture at reflux overnight. Cool the mixture, filter the solids, and concentrate the filtrate in vacuo. Purify on silica gel eluting with 15% to 50% acetone/hexanes to give benzyl-methyl-(2-oxiranyl-ethyl)-amine (27.07 g, 69%). Mass spectrum (m/z): m/z=192 (M+1), ¹H NMR (CDCl₃) δ 7.31 (d, 4H), 7.29-7.22 (m, 1H), 3.50 (s, 2H), 3.01-2.97 (m, 1H), 2.77-2.54 (m, 2H), 2.50-2.48 (m, 1H), 2.20 (s, 3H), 1.81-1.67 (m, 2H).

### Preparation 40

### (S)-Benzyl-methyl-(2-oxiranyl-ethyl)-amine

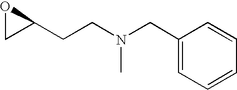

Using a method similar to Preparation 39, using (S)-(−)-4-bromo-1,2-epoxybutane affords the title compound. Mass spectrum (m/z): m/z=192 (M+1), ¹H NMR (CDCl₃) δ 7.31 (d, 4H), 7.29-7.22 (m, 1H), 3.50 (s, 2H), 3.01-2.97 (m, 1H), 2.77-2.75 (m, 1H), 2.57-2.54 (m, 2H), 2.50-2.48 (m, 1H), 2.20 (s, 3H), 1.81-1.66 (m, 2H).

### Preparation 41

### (R)-4-(Benzyl-methyl-amino)-1-morpholin-4-yl-butan-2-ol

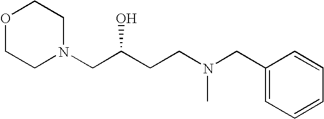

Add morpholine (0.35 mL, 4.01 mmol) to a solution of (R)-benzyl-methyl-(2-oxiranyl-ethyl)-amine (0.306 g, 1.60 mmol) in methanol (3 mL) and heat at reflux overnight. Cool to ambient temperature and concentrate in vacuo. Purify the residue on silica gel eluting with 0.45% NH₄OH/4.5% ethanol/chlorofom to give (R)-4-(benzyl-methyl-amino)-1-morpholin-4-yl-butan-2-ol (0.292 g, 66%). Mass spectrum (ion spray): m/z=279 (M+1), ¹H NMR (CDCl₃) δ 7.35-7.21 (m, 5H), 5.42 (brs, 1H), 3.93-3.85 (m, 1H), 3.73-3.68 (m, 4H), 3.52 (dd, 2H), 2.76-2.66 (1H), 2.60-2.50 (m, 3H), 2.48-2.34 (m, 3H), 2.29-2.23 (m, 1H), 2.21 (s, 3H), 1.74-1.55 (m, 2H).

### Preparation 42

### (R)-4-(Benzyl-methyl-amino)-1-pyrrolidin-1-yl-butan-2-ol

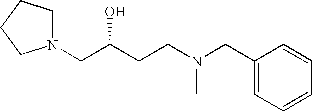

Add pyrrolidine (5.0 mL, 59.9 mmol) to (R)-benzyl-methyl-(2-oxiranyl-ethyl)-amine (0.303 g, 1.58 mmol) and stir at ambient temperature for 3 days before concentrating in vacuo. Purify the residue on silica gel eluting with 1.0% NH₄OH/10.0% ethanol/chlorofom to give (R)-4-(benzyl-methyl-amino)-1-morpholin-4-yl-butan-2-ol (0.343 g, 82%). Mass spectrum (ion spray): m/z=263 (M+1), ¹H NMR (CDCl₃) δ 7.35-7.21 (mn, 5H), 5.45 (brs, 1H), 290-3.81 (m, 1H), 3.51 (dd, 2H), 2.75-2.65 (m, 1H), 2.64-2.47 (m, 6H), 2.36 (dd, 1H), 2.20 (s, 3H), 1.80-1.58 (m, 6H).

### Preparation 43

### (R)-4-(Benzyl-methyl-amino)-1-isopropylsulfanyl-butan-2-ol

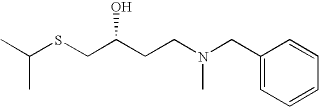

Add 2-propanethiol (0.550 mL, 5.92 mmol) to a solution of (R)-benzyl-methyl-(2-oxiranyl-ethyl)-amine (1.01 g, 5.28 mmol) and triethylamine (0.820 mL, 5.88 mmol) in methanol (20 mL) and stir at ambient temperature overnight. Concentrate the reaction mixture in vacuo and purify the residue on silica gel eluting with 0.275% NH₄OH/2.75% ethanol/chloroform to give (R)-benzyl[3-(2,4-dichloro-phenoxy)-4-isopropylsulfanyl-butyl]-methyl-amine (1.16 g, 83%). Mass spectrum (ion spray): m/z=268 (M+1), ¹H NMR (CDCl₃) δ 7.38-7.27 (mn, 5H), 6.35 (brs, 1H), 3.97-3.87 (m, 1H), 3.57 (dd, 2H), 3.03-2.92 (m, 1H), 2.83-2.50 (m, 4H), 2.26 (s, 3H), 1.83-1.71 (m, 2H), 1.29 (dd, 6H).

### Preparation 44

### (R)-4-(Benzyl-methyl-amino)-1-tert-butoxy-butan-2-ol

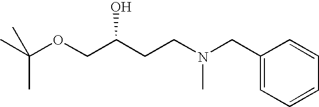

Add KOtBu (1.72 mL, 1.72 mmol) to a solution of (R)-benzyl-methyl-(2-oxiranyl-ethyl)-amine (0.164 g, 0.86 mmol) in tert-butanol (3 mL) and heat at 70° C. for 6 hours. Cool the reaction mixture to ambient temperature overnight, then return to reflux for 5 hours. Cool the reaction mixture to ambient temperature and add water. Load solution onto an SCX column, wash with methanol, and recover amine material by eluting with 2N NH₃ in methanol. Concentrate the filtrate in vacuo, and purify the residue on silica gel eluting with 2.5% 2N NH₃ in methanol/dichloromethane to give (R)-4-(benzyl-methyl-amino)-1-tert-butoxy-butan-2-ol (0.075 g, 33%). Mass spectrum (ion spray): m/z=266 (M+1), ¹H NMR (CD₃OD) δ 7.32-7.23 (m, 5H), 3.72-3.67 (m, 1H), 3.53 (dd, 2H), 3.43-3.24 (m, 2H), 2.68-2.61 (m, 1H), 2.57-2.50 (m, 1H), 2.21 (s, 3H), 1.80-1.60 (m, 2H), 1.18 (s, 9H).

A method similar to that described in Preparation 44 is used to prepare the following compounds:

| | | | | Mass | |
|---|---|---|---|---|---|
| | | | | Spectrum (ion | |
| | | Base/ | Reagent | spray) m/z | |
| Preparation | Name | Solvent | Enantiomer | (M+1) | ¹H NMR |
| 45 | (S)-4-(Benzyl- | KOtBu/ | (S) | 266 | (CD₃OD) δ 7.32-7.23(m, |
| | methyl- | tert- | | | 5H,), 3.72-3.67(m, 1H), |
| | amino)-1-tert- | butanol | | | 3.53(dd, 2H), |
| | butoxy-butan- | | | | 3.43-3.24(m, 2H), 2.68-2.61(m, |
| | 2-ol | | | | 1H), 2.57-2.50(m, 1H), |
| | | | | | 2.21(s, 3H), 1.80-1.60(m, |
| | | | | | 2H), 1.18(s, 9H) |
| 46 | (S)-4-(Benzyl- | NaOMe/ | (S) | 224 | (CDCl₃) δ 7.34-7.22(m, |
| | methyl- | methanol | | | 5H), 6.00(brs, 1H), |
| | amino)-1- | | | | 3.96-3.88(m, 1H), 3.51(dd, |
| | methoxy- | | | | 2H), 3.38-3.25(m, 5H), |
| | butan-2-ol | | | | 2.87-2.69(m, 1H), |
| | | | | | 2.61-2.54(m, 1H), 2.21(s, 3H), |
| | | | | | 1.79-1.67(m, 1H), |
| | | | | | 1.63-1.54(m, 1H) |
| 47 | (S)-4-(Benzyl- | NaOEt/ | (S) | 238 | (CDCl₃) δ 7.34-7.21(m, |
| | methyl- | ethanol | | | 5H), 5.93(brs, 1H), |
| | amino)-1- | | | | 3.97-3.88(m, 1H), 3.60(d, 1H), |
| | ethoxy-butan- | | | | 3.54-3.48(m, 2H), |
| | 2-ol | | | | 3.45-3.37(m, 2H), |
| | | | | | 3.35-3.29(m, 1H), 2.28-2.69(m, |
| | | | | | 1H), 2.61-2.54(m, 1H), |
| | | | | | 2.21(s, 3H), 1.77-1.59(m, |
| | | | | | 2H), 1.19(t, 3H) |
| 48 | (R)-4-(Benzyl- | NaOiBu/ | (R) | 266 | (CDCl₃) δ 7.35-7.21(m, |
| | methyl- | isobutanol | | | 5H), 5.85(brs, 1H), |
| | amino)-1- | | | | 3.96-3.89(m, 1H), 3.60(d, 1H), |
| | isobutoxy- | | | | 3.47-3.37(m, 2H), |
| | butan-2-ol | | | | 3.32-3.27(m, 1H), |
| | | | | | 3.25-3.16(m, 2H), 2.77-2.69(m, |
| | | | | | 1H), 2.63-2.56(m, 1H), |
| | | | | | 2.21(s, 3H), 1.92-1.80(m, |
| | | | | | 1H), 1.78-1.61(m, 2H), |
| | | | | | 0.88(d, 6H) |
| 49 | (R)-4-(Benzyl- | NaOiPr/ | (R) | 252 | (CDCl₃) δ 7.36-7.21(m, |
| | methyl- | isopropanol | | | 5H), 5.81(brs, 1H), |
| | amino)-1- | | | | 3.93-3.83(m, 1H), |
| | isopropoxy- | | | | 3.63-3.51(m, 2H), 3.47-3.35(m, |
| | butan-2-ol | | | | 2H), 3.31-3.24(m, 1H), |
| | | | | | 2.77-2.67(m, 1H), |
| | | | | | 2.61-2.53(m, 1H), 2.21(s, 3H), |
| | | | | | 1.75-1.62(m, 2H), |
| | | | | | 1.14(dd, 6H) |

### Preparation 50

### (R)-(3-Hydroxy-4-morpholin-4-yl-butyl)-methyl-carbamic acid tert-butyl ester

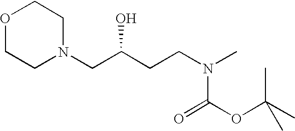

Add 10% Pd/C (0.028 g) to a solution of (R)-4-(benz,yl-methyl-amino)-1-morpholin-4-yl-butan-2-ol (0.284 g, 1.02 mmol) and di-tert-butyl dicarbonate (0.223 g, 1.02 mmol) in ethanol (25 mL). Pressurize to 60 psi of hydrogen and stir the reaction mixture overnight at ambient temperature. Filter the mixture through Celite® and concentrate the filtrate in vacuo.

Purify on silica gel eluting with 0.45% NH₄OH/4.5% ethanol/chloroform to give (R)-(3-hydroxy-4-morpholin-4-yl-butyl)-methyl-carbamic acid tert-butyl ester (0.257 g, 87%): Mass spectrum (ion spray): m/z=289 (M+1), ¹H NMR (CDCl₃) δ 3.77-3.67 (m, 5H), 3.40-3.28 (brs, 2H), 2.86 (s, 3H), 2.58-2.28 (m, 5H), 1.74 (brs, 1H), 1.51 (s, 3H), 1.45 (s, 9H).

### Preparation 51

### (R)-(3-Hydroxy-4-pyrrolidin-1-yl-butyl)-methyl-carbamic acid tert-butyl ester

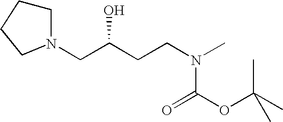

Add 10% Pd/C (0.033 g) to a solution of (R)-4-(benzyl-methyl-amino)-1-pyrrolidine-1-yl-butan-2-ol (0.331 g, 1.26 mmol) and di-tert-butyl dicarbonate (0.259 g, 1.19 mmol) in ethanol (25 mL). Pressurize to 60 psi of hydrogen and stir the reaction mixture overnight at ambient temperature. Filter the mixture through Celite® and concentrate the filtrate in vacuo. Purify the residue on silica gel eluting with 1.0% NH₄OH/10.0% ethanol/chlorofom to give (R)-(3-hydroxy-4-pyrrolidin-1-yl-butyl)-methyl-carbamic acid tert-butyl ester (0.204 g, 63%). Mass spectrum (ion spray): m/z=273 (M+1), ¹H NMR (CDCl₃) δ 4.84 (s, 1H), 3.74-3.65 (m, 1H), 3.43-3.28 (brm, 2H), 2.86 (s, 3H), 2.63-2.45 (m, 6H), 1.82-1.72 (m, 5H), 1.56-1.47 (brm, 1H), 1.45 (s, 9H).

### Preparation 52

### 4-Chloro-2-trifluoromethyl-phenol

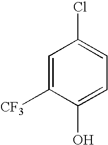

Add a solution of NaNO₂ (0.458 g, 6.63 mmol) in water (2 mL) to a cool (0° C.) mixture of 4-chloro-2-trifluoromethyl-aniline (1.08 g, 5.52 mmol) in 33% H₂SO₄ (40 mL). After 3 hours, a urea (0.100 g, 1.67 mmol) and stir for 10 minutes. Add this reaction mixture to 100 mL of refluxing 33% H₂SO₄ and heat at reflux for 1 hour, before cooling to ambient temperature. Extract the mixture with EtOAc. Wash the organic extracts with water and aqueous saturated sodium chloride, dry over anhydrous Na₂SO₄, filter, and concentrate in vacuo. Purify on silica gel eluting with 20% EtOAc/hexanes to give 4-chloro-2-trifluoromethyl-phenol (0.245 g, 23%). Mass spectrum (ion spray): m/z=195 (M−1), ¹H NMR (CDCl₃) δ 7.49 (d, 1H), 7.38 (dd, 1H), 6.91 (d, 1H), 5.44 (brs, 1H).

### Preparation 53

### 3,4,5-Trichloro benzenediazonium tetrafluoroborate

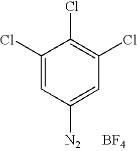

Add borontrifluoride diethyl etherate (10.0 mL, 78.9 mmol, 1.6 equiv.) and dry diethyl ether ether (250 mL) to a cold (−20°) stirred solution of 2,4,5-trichloroaniline (10.00 g, 50.90 mmol, 1 equiv.) in dry dichloromethane (400 mL). Add tert-butyl nitrite (10.0 mL, 84.1 mmol, 1.7 equiv.) dropwise over 5 minutes and stir at −20° C. for 25 minutes before warming to room temperature and stirring an additional 20 minutes. The reaction mixture is concentrated under reduced pressure and the acquired solid is suspended in hexanes. The mixture is filtered, the collected solid is washed with hexanes, and cold diethyl ether, and air dried to afford the title compound as a tan solid which was used without farther purification. (13.00 g, 87%). ¹H NMR (DMSO) δ 9.04 (s, 2H).

### Preparation 54

### 3,4,5-Trichlorophenol

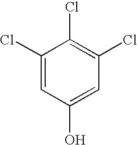

Add solid 3,4,5-trichloro benzenediazonium tetrafluoroborate (9.62 g, 32.6 mmol, 1 equiv.) to a room temperature solution of copper (II) nitrate trihydrate (450 g, 1.86 mol, 57 equiv.) and copper (I) oxide (14.03 g, 98.1 mmol, 3 equiv.) in water (3.5 L) and vigorously stir the reaction mixture at room temperature for 1 h10 min. The solids are filtered and then washed with dichloromethane. Extract the filtrate three times with dichloromethane, dry over anhydrous magnesium sulfate, filter, and concentrate under reduced pressure. Purify on silica gel eluting with 0 to 20% EtOAc/hexanes to give the title compound as an orange brown solid (3.199 g, 49%). ¹H NMR (CDCl₃) δ 6.91 (s, 2H), 4.95 (br s, 1H).

### Preparation 55

### (S)-Benzyl-[3-(3-chloro-phenoxy)-hexyl]-methyl-amine

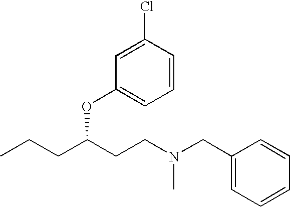

Add 1,1′-azodicarbonyl-dipiperdine (0.590 g, 2.34 mmol) to a stirred solution of (R)-1-(benzyl-methyl-amino)-hexan-3-ol (0.432 g, 1.95 mmol), 3-chlorophenol (0.301 g, 2.34 mmol), and tri-n-butylphosphine (0.473 g, 2.34 mmol) in toluene (15 mL). Heat the reaction mixture at 65° C. overnight, then cool the solution to ambient temperature, and add dichloromethane. Filter the solids and concentrate the filtrate in vacuo. Purify the residue on silica gel eluting with 20% EtOAc/dichloromethane) to give (S)-benzyl-[3-(3-chloro-phenoxy)-hexyl]-methyl-amine (0.516 g, 80%). Mass spectrum (ion spray): m/z=333 (M+1), ¹H NMR (CDCl₃) δ 7.32-7.21 (m, 6H), 7.18-7.13 (m, 1H), 6.95-6.92 (m, 1H), 6.91-6.87 (m, 1H), 6.80-6.76 (m, 1H), 4.41-4.32 (m, 1H), 3.48 (s, 2H), 2.55-2.41 (m, 2H), 2.20 (s, 3H), 1.92-1.76 (m, 2H), 1.68-1.26 (m, 4H), 0.92 (t, 3H).

A method similar to that described in Preparation 55 is used to prepare the following compounds:

| | | | Mass | |
|---|---|---|---|---|
| | | | Spectrum | |
| | | Reagent | (ion spray) | |
| Preparation | Name | Enantiomer | m/z (M+1) | ¹H NMR |
| 56 | (S)-Benzyl-[3-(4-chloro- | (R) | 400 | (CD₃OD) δ 7.42(d, |
| | 3-trifluoromethyl- | | | 1H), 7.24-7.19(m, |
| | phenoxy)-hexyl]-methyl- | | | 6H), 7.09(dd, 1H), |
| | amine | | | 4.47-4.42(m, 1H), |
| | | | | 3.47(d, 2H), |
| | | | | 2.50-2.40(m, 2H), 2.21(s, |
| | | | | 3H), 1.87-1.81(m, |
| | | | | 2H), 1.65-1.50(m, |
| | | | | 2H), 1.46-1.29(m, |
| | | | | 2H), 0.91(t, 3H) |
| 57 | (S)-Benzyl-[3-(2-chloro- | (R) | 350 | (CDCl₃) δ |
| | 4-fluoro-phenoxy)- | | | 7.31-7.21(m, 5H), 7.09(dd, |
| | hexyl]-methyl-amine | | | 1H), 6.95(dd, 1H), |
| | | | | 6.90-6.84(m, 1H), |
| | | | | 4.39-4.32(m, 1H), |
| | | | | 3.45(s, 2H), |
| | | | | 2.63-2.43(m, 2H), 2.18(s, |
| | | | | 3H), 1.96-1.80(m, |
| | | | | 2H), 1.76-1.36(m, |
| | | | | 4H), 0.92(t, 3H) |
| 58 | (S)-Benzyl-[3-(2-chloro- | (R) | 400 | (CDCl₃) δ 7.59(d, |
| | 4-trifluoromethyl- | | | 1H), 7.42(dd, 1H), |
| | phenoxy)-hexyl]-methyl- | | | 7.28-7.19(m, 5H), |
| | amine | | | 7.06(d, 1H), |
| | | | | 4.56-4.49(m, 1H), |
| | | | | 3.47(brs, 2H), |
| | | | | 2.61-2.41(m, 2H), 2.20(s, 3H), |
| | | | | 1.99-1.82(m, 2H), |
| | | | | 1.76-1.55(m, 2H), |
| | | | | 1.50-1.30(m, 2H), |
| | | | | 0.92(t, 3H) |
| 59 | (S)-Benzyl-[3-(4-fluoro- | (R) | 366 | (CDCl₃) δ 8.20(d, |
| | naphthalen-1-yloxy)- | | | 1H), 8.03(d, 1H), |
| | hexyl]-methyl-amine | | | 7.56-7.47(m, 2H), |
| | | | | 7.28-7.18(m, 5H), |
| | | | | 7.03-6.98(m, 1H), |
| | | | | 6.76(dd, 1H), |
| | | | | 4.60-4.53(m, 1H), 3.48(s, |
| | | | | 2H), 2.60-2.53(m, |
| | | | | 2H), 2.20(s, 3H), |
| | | | | 2.08-1.89(m, 2H), |
| | | | | 1.83-1.62(m, 2H), |
| | | | | 1.55-1.38(m, 2H), |
| | | | | 0.93(t, 3H) |
| 60 | (S)-Benzyl-[3-(2,3- | (R) | 348 | (CDCl₃) δ |
| | difluoro-4-methyl- | | | 7.31-7.20(m, 5H), 6.77(dd, |
| | phenoxy)-hexyl]-methyl- | | | 1H), 6.69(dd, 1H), |
| | amine | | | 4.39-4.31(m, 1H), |
| | | | | 3.48(s, 2H), |
| | | | | 2.59-2.44(m, 2H), 2.23(s, |
| | | | | 3H), 2.18(s, 3H), |
| | | | | 1.96-1.78(m, 2H), |
| | | | | 1.71-1.51(m, 2H), |
| | | | | 1.49-1.35(m, 2H), |
| | | | | 0.92(t, 3H) |
| 61 | (R)-Benzyl-[3-(4-fluoro- | (S) | 366 | (CDCl₃) δ 8.20(d, |
| | naphthalen-1-yloxy)- | | | 1H), 8.02(d, 1H), |
| | hexyl]-methyl-amine | | | 7.56-7.46(m, 2H), |
| | | | | 7.28-7.18(m, 5H), |
| | | | | 7.00(dd, 1H), |
| | | | | 6.75(dd, 1H), |
| | | | | 4.58-4.54(m, 1H), 3.49(brs, |
| | | | | 2H), 2.60-2.53(m, |
| | | | | 2H), 2.18(s, 3H), |
| | | | | 2.08-1.90(m, 2H), |
| | | | | 1.81-1.61(m, 2H), |
| | | | | 1.54-1.40(m, 2H), |
| | | | | 0.93(t, 3H) |
| 62 | Benzyl-[3-(2,4-dichloro- | Separated | 420 | (CD₃OD) δ 7.38(d, |
| | phenoxy)-6,6,6-trifluoro- | Racemic | | 1H), 7.27-7.21(m, |
| | hexyl]-methyl-amine | Mixture | | 6H), 7.08(d, 1H), |
| | Enantiomer 1 | | | 4.57-4.54(m, 1H), |
| | | | | 3.49(s, 2H), |
| | | | | 2.53-2.45(m, 2H), |
| | | | | 2.35-2.21(m, 2H), 2.20(s, |
| | | | | 3H), 1.95-1.83(m, |
| | | | | 4H) |
| 63 | (S)-Benzyl-methyl-[3-(2- | (R) | 366 | (CDCl₃) δ 7.55(d, |
| | trifluoromethyl- | | | 1H), 7.43(dd, 1H), |
| | phenoxy)-hexyl]-amine | | | 7.27-7.20(m, 5H), |
| | | | | 7.06(d, 1H), |
| | | | | 6.94(dd, 1H), |
| | | | | 4.58-4.55(m, 1H), 3.47(s, 2H), |
| | | | | 2.58-2.44(m, 2H), |
| | | | | 2.20(s, 3H), |
| | | | | 1.96-1.83(m, 2H), |
| | | | | 1.73-1.56(m, 2H), |
| | | | | 1.49-1.38(m, 2H), 0.92(t, |
| | | | | 3H) |
| 64 | (S)-Benzyl-methyl-[3-(4- | (R) | 366 | (CDCl₃) δ 7.50(d, |
| | trifluoromethyl- | | | 2H), 7.29-7.19(m, 5), |
| | phenoxy)-hexyl]-amine | | | 6.85(d, 2H), |
| | | | | 4.49-4.29(m, 1H), 3.48(s, |
| | | | | 1H), 2.53-2.42(m, |
| | | | | 2H), 2.20(s, 3H), |
| | | | | 1.92-1.79(m, 2H), |
| | | | | 1.68-1.33(m, 4H), |
| | | | | 0.92(t, 3H) |
| 65 | (S)-Benzyl-[3-(2-chloro- | (R) | 332 | (CDCl₃) δ 7.34(d, |
| | phenoxy)-hexyl]-methyl- | | | 1H), 7.28-7.21(m, |
| | amine | | | 5H), 7.17(dd, 1H), |
| | | | | 7.02(d, 1H), |
| | | | | 6.85(dd, 1H), |
| | | | | 4.47-4.44(m, 1H), 3.49(s, 2H), |
| | | | | 2.60-2.50(m, 2H), |
| | | | | 2.20(s, 3H), |
| | | | | 1.97-1.86(m, 2H), |
| | | | | 1.74-1.4(m, 4H), 0.93(t, |
| | | | | 3H) |
| 66 | (S)-Benzyl-[3-(4-chloro- | (R) | 332 | (CDCl₃) δ |
| | phenoxy)-hexyl]-methyl- | | | 7.29-7.2(m, 5H), 7.19(d, 2H), |
| | amine | | | 6.83(d, 2H), |
| | | | | 4.38-4.31(m, 1H), 3.35(s, |
| | | | | 3H), 2.57-2.43(m, |
| | | | | 2H), 2.20(s, 3H), |
| | | | | 1.92-1.78(m, 2H), |
| | | | | 1.68-1.32(m, 4H), |
| | | | | 0.92(t, 3H) |
| 67 | (S)-Benzyl-[3-(2,3- | (R) | 366 | (CDCl₃) δ |
| | dichloro-phenoxy)-hexyl]- | | | 7.28-7.19(m, 5H), 7.08(dd, |
| | methyl-amine | | | 1H), 7.02(d, 1H), |
| | | | | 6.92(d, 1H), |
| | | | | 4.46-4.43(m, 1H), |
| | | | | 3.50-3.43(m 2H), |
| | | | | 2.59-2.42(m, 2H), 2.19(s, |
| | | | | 3H), 1.94-1.82(m, |
| | | | | 2H), 1.70-1.54(m, |
| | | | | 2H), 1.47-1.37(m, |
| | | | | 2H), 0.92(t, 3H) |
| 68 | (S)-Benzyl-[3-(naphthalen- | (R) | 348 | (CDCl₃) δ |
| | 2-yloxy)-hexyl]-methyl- | | | 7.77-7.68(m, 3H), 7.42(dd, |
| | amine | | | 1H), 7.34-7.2(m, |
| | | | | 7H), 7.14(d, 1H), |
| | | | | 4.61-4.55(m, 1H), |
| | | | | 3.51(s, 3H), |
| | | | | 2.60-2.55(m, 2H), 2.20(s, |
| | | | | 3H), 2.04-2.86(m, |
| | | | | 2H), 1.78-1.61(m, |
| | | | | 2H), 1.57-1.41(m, |
| | | | | 2H), 0.95(t, 3H) |
| 69 | (S)-Benzyl-[3- | (R) | 347(GC- | (CDCl₃) δ 8.24(d, |
| | (naphthalen-1-yloxy)- | | Mass) | 1H), 7.79(d, 1H), |
| | hexyl]-methyl-amine | | | 7.50-7.32(m, 4H), |
| | | | | 7.30-7.19(m, 5H), |
| | | | | 6.89(d, 1H), |
| | | | | 4.67-4.61(m, 1H), 3.49(s, |
| | | | | 2H), 2.58(t, 2H), |
| | | | | 2.20(s, 3H), |
| | | | | 2.10-1.93(m, 2H), |
| | | | | 1.85-1.65(m, 2H), |
| | | | | 1.58-1.42(m, 2H), 0.94(t, |
| | | | | 3H) |
| 70 | (S)-Benzyl-[3-(2-chloro- | (R) | 400 | (CDCl₃) δ |
| | 3-trifluoromethyl- | | | 7.29-7.18(m, 8H), |
| | phenoxy)-hexyl]-methyl- | | | 4.58-4.43(m, 1H), |
| | amine | | | 3.51-3.42(m, 2H), |
| | | | | 2.61-2.42(m, 2H), 2.20(s, 3H), |
| | | | | 1.90-1.82(m, 2H), |
| | | | | 1.76-1.58(m, 2H), |
| | | | | 1.52-1.37(m, 2H), |
| | | | | 0.95(t, 3H) |
| 71 | (S)-Benzyl-[3-(2,3,5- | (R) | 402 | (CDCl₃) δ |
| | trichloro-phenoxy)- | | | 7.29-7.2(m, 5H), 7.05(s, 1H), |
| | hexyl]-methyl-amine | | | 7.02(s, 1H), |
| | | | | 4.43-4.40(m, 1H), |
| | | | | 3.52-3.44(m, 2H), |
| | | | | 2.57-2.51(m, 1H), |
| | | | | 2.45-2.39(m, 1H), 2.20(s, |
| | | | | 3H), 1.93-1.79(m, |
| | | | | 2H), 1.72-1.35(m, |
| | | | | 4H), 0.92(t, 3H) |
| 72 | (R)-Benzyl-[3-(4-chloro- | (S) | 332 | (CDCl₃) δ |
| | phenoxy)-hexyl]-methyl- | | | 7.29-7.20(m, 5H), 7.19(d, 2H), |
| | amine | | | 6.83(d, 2H), |
| | | | | 4.38-4.31(m, 1H), 3.35(s, |
| | | | | 3H), 2.57-2.43(m, |
| | | | | 2H), 2.20(s, 3H), |
| | | | | 1.92-1.78(m, 2H), |
| | | | | 1.68-1.32(m, 4H), |
| | | | | 0.92(t, 3H) |
| 73 | (R)-Benzyl-[3-(2,3- | (S) | 366 | (CDCl₃) δ |
| | dichloro-phenoxy)- | | | 7.28-7.19(m, 5H), 7.08(dd, |
| | hexyl]-methyl-amine | | | 1H), 7.02(d, 1H), |
| | | | | 6.92(d, 1H), |
| | | | | 4.46-4.43(m, 1H), |
| | | | | 3.50-3.43(m, 2H), |
| | | | | 2.59-2.42(m, 2H), 2.19(s, |
| | | | | 3H), 1.94-1.82(m, |
| | | | | 2H), 1.70-1.54(m, |
| | | | | 2H), 1.47-1.37(m, |
| | | | | 2H), 0.92(t, 3H) |
| 74 | (R)-Benzyl-[3- | (S) | 348 | (CDCl₃) δ |
| | (naphthalen-2-yloxy)- | | | 7.77-7.68(m, 3H), 7.42(dd, |
| | hexyl]-methyl-amine | | | 1H), 7.34-7.20(m, |
| | | | | 7H), 7.14(d, 1H), |
| | | | | 4.61-4.55(m, 1H), |
| | | | | 3.51(s, 3H), |
| | | | | 2.60-2.55(m, 2H), 2.20(s, |
| | | | | 3H), 2.04-2.86(m, |
| | | | | 2H), 1.78-1.61(m, |
| | | | | 2H), 1.57-1.41(m, |
| | | | | 2H), 0.95(t, 3H) |
| 75 | (R)-Benzyl-[3- | (S) | 347(GC- | (CDCl₃) δ 8.24(d, |
| | (naphthalen-1-yloxy)- | | Mass) | 1H), 7.79(d, 1H), |
| | hexyl]-methyl-amine | | | 7.50-7.32(m, 4H), |
| | | | | 7.30-7.19(m, 5H), |
| | | | | 6.89(d, 1H), |
| | | | | 4.67-4.61(m, 1H), 3.49(s, |
| | | | | 2H), 2.58(t, 2H), |
| | | | | 2.20(s, 3H), |
| | | | | 2.10-1.93(m, 2H), |
| | | | | 1.85-1.65(m, 2H), |
| | | | | 1.58-1.42(m, 2H), 0.94(t, |
| | | | | 3H) |
| 76 | (R)-Benzyl-[3-(2-chloro- | (S) | 400 | (CDCl₃) δ |
| | 3-trifluoromethyl- | | | 7.29-7.18(m, 8H), |
| | phenoxy)-hexyl]-methyl- | | | 4.58-4.43(m, 1H), |
| | amine | | | 3.51-3.42(m, 2H), |
| | | | | 2.61-2.42(m, 2H), 2.20(s, 3H), |
| | | | | 1.90-1.82(m, 2H), |
| | | | | 1.76-1.58(m, 2H), |
| | | | | 1.52-1.37(m, 2H), |
| | | | | 0.95(t, 3H) |
| 77 | (R)-Benzyl-methyl-[3- | (S) | 402 | (CDCl₃) δ |
| | (2,3,5-trichloro- | | | 7.29-7.20(m, 5H), 7.05(s, 1H), |
| | phenoxy)-hexyl]-amine | | | 7.02(s, 1H), |
| | | | | 4.43-4.40(m, 1H), |
| | | | | 3.52-3.44(m, 2H), |
| | | | | 2.57-2.51(m, 1H), |
| | | | | 2.45-2.39(m, 1H), 2.20(s, |
| | | | | 3H), 1.93-1.79(m, |
| | | | | 2H), 1.72-1.35(m, |
| | | | | 4H), 0.92(t, 3H) |
| 78 | (S)-Benzyl-[3-(2,4- | (R) | 366 | (CDCl₃) δ |
| | dichloro-phenoxy)- | | | 7.33-7.21(m, 6H), 7.12(dd, |
| | hexyl]-methyl-amine | | | 1H), 6.94(d, 1H), |
| | | | | 4.43-4.36(m, 1H), |
| | | | | 3.46(s, 2H), |
| | | | | 2.58-2.40(m, 2H), 2.17(s, |
| | | | | 3H), 1.93-1.78(m, |
| | | | | 2H), 1.71-1.52(m, |
| | | | | 2H), 1.48-1.33(m, |
| | | | | 2H), 0.90(t, 3H) |
| 79 | (S)-Benzyl-[3-(3,4- | (R) | 366 | (CDCl₃) δ |
| | dichloro-phenoxy)- | | | 7.31-7.21(m, 6H), 7.04(d, 1H), |
| | hexyl]-methyl-amine | | | 6.74(dd, 1H), |
| | | | | 4.36-4.29(m, 1H), |
| | | | | 3.46(dd, 2H), |
| | | | | 2.51-2.37(m, 2H), 2.19(s, 3H), |
| | | | | 1.86-1.71(m, 2H), |
| | | | | 1.65-1.28(m, 4H), |
| | | | | 0.89(t, 3H) |
| 80 | (R)-Benzyl-[3-(3,4- | (S) | 366 | (CDCl₃) δ |
| | dichloro-phenoxy)- | | | 7.31-7.21(m, 6H), 7.04(d, 1H), |
| | hexyl]-methyl-amine | | | 6.74(dd, 1H), |
| | | | | 4.36-4.29(m, 1H), |
| | | | | 3.46(dd, 2H), |
| | | | | 2.51-2.37(m, 2H), 2.19(s, 3H), |
| | | | | 1.86-1.71(m, 2H), |
| | | | | 1.65-1.28(m, 4H), |
| | | | | 0.89(t, 3H) |
| 81 | (S)-Benzyl-[3-(3,5- | (R) | 366 | (CDCl₃) δ |
| | dichloro-phenoxy)- | | | 7.30-7.20(m, 6H), 6.91(dd, |
| | hexyl]-methyl-amine | | | 1H), 6.82(d, 1H), |
| | | | | 4.38-4.31(m, 1H), |
| | | | | 3.46(dd, 2H), |
| | | | | 2.50-2.35(m, 2H), 2.19(s, |
| | | | | 3H), 1.86-1.72(m, |
| | | | | 2H), 1.63-1.28(m, |
| | | | | 4H), 0.89(t, 3H) |
| 82 | (R)-Benzyl-[3-(3,5- | (S) | 366 | (CDCl₃) δ |
| | dichloro-phenoxy)- | | | 7.30-7.20(m, 6H), 6.91(dd, |
| | hexyl]-methyl-amine | | | 1H), 6.82(d, 1H), |
| | | | | 4.38-4.31(m, 1H), |
| | | | | 3.46(dd, 2H), |
| | | | | 2.50-2.35(m, 2H), 2.19(s, |
| | | | | 3H), 1.86-1.72(m, |
| | | | | 2H), 1.63-1.28(m, |
| | | | | 4H), 0.89(t, 3H) |
| 83 | (S)-Benzyl-[3-(2,4- | (R) | 380 | (CDCl₃) δ |
| | dichloro-6-methyl- | | | 7.33-7.22(m, 5H), 7.18(d, 1H), |
| | phenoxy)-hexyl]-methyl- | | | 7.03(d, 1H), |
| | amine | | | 4.44-4.38(m, 1H), |
| | | | | 3.45(dd, 2H), |
| | | | | 2.52-2.46(m, 2H), 2.24(s, 3H), |
| | | | | 2.14(s, 3H), |
| | | | | 1.87-1.80(m, 2H), |
| | | | | 1.61-1.33(m, 4H), 0.89(t, |
| | | | | 3H) |
| 84 | (S)-Benzyl-[3-(4-chloro- | (R) | 360 | (CDCl₃) δ |
| | 3,5-dimethyl-phenoxy)- | | | 7.31-7.21(m, 5H), 6.65(s, 2H), |
| | hexyl]-methyl-amine | | | 4.36-4.29(m, 1H), |
| | | | | 3.47(s, 2H), |
| | | | | 2.50-2.44(m, 2H), 2.32(s, |
| | | | | 6H), 2.18(s, 3H), |
| | | | | 1.89-1.72(m, 2H), |
| | | | | 1.64-1.31(m, 4H), |
| | | | | 0.90(m, 3H) |
| 85 | (R)-Benzyl-[3-(4-chloro- | (S) | 360 | (CDCl₃) δ |
| | 3,5-dimethyl-phenoxy)- | | | 7.31-7.21(m, 5H), 6.65(s, 2H), |
| | hexyl]-methyl-amine | | | 4.36-4.29(m, 1H), |
| | | | | 3.47(s, 2H), |
| | | | | 2.50-2.44(m, 2H), 2.32(s, |
| | | | | 6H), 2.18(s, 3H), |
| | | | | 1.89-1.72(m, 2H), |
| | | | | 1.64-1.31(m, 4H), |
| | | | | 0.90(m, 3H) |
| 86 | (R)-Benzyl-[3-(2,4- | (S) | 368 | (CDCl₃) δ |
| | dichloro-phenoxy)-4- | | | 7.32-7.18(m, 6H), |
| | methoxy-butyl]-methyl- | | | 7.15-7.05(m, 2H), |
| | amine | | | 4.56-4.49(m, 1H), |
| | | | | 3.59-3.48(m, 2H), 3.44(s, 2H), |
| | | | | 3.35(s, 3H), |
| | | | | 2.64-2.55(m, 1H), |
| | | | | 2.49-2.41(m, 1H), 2.15(s, |
| | | | | 3H), 1.93-1.86(m, |
| | | | | 2H) |
| 87 | (R)-Benzyl-[-3-(2,4- | (S) | 382 | (CDCl₃) δ |
| | dichloro-phenoxy)-4- | | | 7.33-7.18(m, 6H), |
| | ethoxy-butyl]-methyl- | | | 7.14-7.08(m, 2H), |
| | amine | | | 4.56-4.48(m, 1H), |
| | | | | 3.63-3.41(m, 6H), |
| | | | | 2.64-2.55(m, 1H), |
| | | | | 2.50-2.42(m, 1H), 2.15(s, 3H), |
| | | | | 1.93-1.86(m, 2H), |
| | | | | 1.15(t, 3H) |
| 88 | Benzyl-[-3-(2,4-dichloro- | Separated | 394 | (CDCl₃) δ |
| | phenoxy)-6-methyl- | Racemic | | 7.33-7.20(m, 6H), 7.11(dd, |
| | heptyl]-methyl-amine | Mixture | | 1H), 6.92(d, 1H), |
| | Enantiomer 2 | | | 4.38-4.32(m, 1H), |
| | | | | 3.45(s, 2H), |
| | | | | 2.57-2.39(m, 2H), 2.17(s, |
| | | | | 3H), 1.93-1.78(m, |
| | | | | 2H), 1.68-1.43(m, |
| | | | | 3H), 1.31-1.15(m, |
| | | | | 2H), 0.86(d, 6H) |
| 89 | Benzyl-[3-(2,3-dichloro- | Separated | 394 | (CDCl₃) δ |
| | phenoxy)-6-methyl- | Racemic | | 7.27-7.18(m, 6H), 7.06(dd, |
| | heptyl]-methyl-amine | Mixture | | 1H), 6.93(dd, 1H), |
| | Enantiomer 1 | | | 4.39-4.34(m, 1H), |
| | | | | 3.42(dd, 2H), |
| | | | | 2.55-2.37(m, 2H), 2.14(s, |
| | | | | 3H), 1.92-1.76(m, |
| | | | | 2H), 1.69-1.42(m, |
| | | | | 3H), 1.30-1.15(m, |
| | | | | 2H), 0.83(d, 6H) |
| 90 | Benzyl-[3-(2,3-dichloro- | Separated | 394 | (CDCl₃) δ |
| | phenoxy)-6-methyl- | Racemic | | 7.27-7.18(m, 6H), 7.06(dd, |
| | heptyl]-methyl-amine | Mixture | | 1H), 6.93(dd, 1H), |
| | Enantiomer 2 | | | 4.39-4.34(m, 1H), |
| | | | | 3.42(dd, 2H), |
| | | | | 2.55-2.37(m, 2H), 2.14(s, |
| | | | | 3H), 1.92-1.76(m, |
| | | | | 2H), 1.69-1.42(m, |
| | | | | 3H), 1.30-1.15(m, |
| | | | | 2H), 0.83(d, 6H) |
| 91 | (S)-Benzyl-[3-(4-chloro- | (R) | 400 | (CDCl₃) δ 7.50(d, |
| | 2-trifluoromethyl- | | | 1H), 7.37(dd, 1H), |
| | phenoxy)-hexyl]-methyl- | | | 7.27-7.20(m, 5H), |
| | amine | | | 7.00(d, 1H), |
| | | | | 4.53-4.47(m, 1H), |
| | | | | 3.44(dd, 2H), |
| | | | | 2.54-2.36(m, 2H), 2.17(s, 3H), |
| | | | | 1.93-1.75(m, 2H), |
| | | | | 1.69-1.51(m, 2H), |
| | | | | 1.46-1.28(m, 2H), |
| | | | | 0.89(t, 3H) |
| 92 | Benzyl-[3-(2,4-dichloro- | not | 338 | (CDCl₃) δ 7.30(d, |
| | phenoxy)-butyl]-methyl- | applicable | | 1H), 7.23-7.10(m, |
| | amine | | | 5H), 7.09-7.08(m, |
| | | | | 1H), 6.85(d, 1H), |
| | | | | 4.47-4.43(m, 1H), |
| | | | | 3.40(s, 3H), |
| | | | | 2.56-2.51(m, 1H), |
| | | | | 2.47-2.42(m, 1H), 2.16(s, |
| | | | | 3H), 1.97-1.92(m, |
| | | | | 1H), 1.78-1.74(m, |
| | | | | 1H), 1.26(d, 3H) |
| 93 | Benzyl-[3-(2,4-dichloro- | not | 352 | (CDCl₃) δ 7.30(d, |
| | phenoxy)-pentyl]-methyl- | applicable | | 1H), 7.27-7.18(m, |
| | amine | | | 5H), 7.11-7.08(m, |
| | | | | 1H), 6.90(d, 1H), |
| | | | | 4.33-4.30(m, 1H), |
| | | | | 3.40(s, 3H), |
| | | | | 2.55-2.50(m, 1H), |
| | | | | 2.45-2.40(m, 1H), 2.15(s, |
| | | | | 3H), 1.90-1.78(m, |
| | | | | 2H), 1.68-1.60(m, |
| | | | | 2H), 0.92(t, 3H) |
| 94 | (S)-Benzyl-[3-(3-chloro- | (R) | 350 | (CDCl₃) δ |
| | 4-fluoro-phenoxy)- | | | 7.31-7.19(m, 5H), 7.08(dd, |
| | hexyl]-methyl-amine | | | 1H), 6.98(dd, 1H), |
| | | | | 6.80(ddd, 1H), |
| | | | | 4.37-4.29(m, 1H), 3.48(s, |
| | | | | 2H), 2.47(ddd, 2H), |
| | | | | 2.20(s, 3H), |
| | | | | 1.86-1.79(m, 2H), |
| | | | | 1.65-1.25(m, 4H), |
| | | | | 0.91(dd, 3H) |
| 95 | (R)-Benzyl-[3-(2,3,4- | (S) | 402 | (CDCl₃) δ |
| | trichloro-phenoxy)- | | | 7.29-7.18(m, 6H), 6.89(d, 1H), |
| | hexyl]-methyl-amine | | | 4.47-4.38(m, 1H), |
| | | | | 3.45(AB_{q}, 2H), |
| | | | | 2.59-2.50(m, 1H), |
| | | | | 2.47-2.38(m, 1H), 2.19(s, |
| | | | | 3H), 1.95-1.78(m, |
| | | | | 2H), 1.73-1.52(m, |
| | | | | 2H), 1.50-1.30(m, |
| | | | | 2H), 0.91(dd, 3H) |
| 96 | (S)-Benzyl-[3-(2,3,4- | (R) | 402 | (CDCl₃) δ |
| | trichloro-phenoxy)- | | | 7.29-7.18(m, 6H), 6.89(d, 1H), |
| | hexyl]-methyl-amine | | | 4.47-4.38(m, 1H), |
| | | | | 3.45(AB_{q}, 2H), |
| | | | | 2.59-2.50(m, 1H), |
| | | | | 2.47-2.38(m, 1H), 2.19(s, |
| | | | | 3H), 1.95-1.78(m, |
| | | | | 2H), 1.73-1.52(m, |
| | | | | 2H), 1.50-1.30(m, |
| | | | | 2H), 0.91(dd, 3H) |
| 97 | (R)-Benzyl-[3-(3,4,5- | (S) | NA | (CDCl₃) δ |
| | trichloro-phenoxy)- | | | 7.32-7.20(m, 5H), 6.98(s, 2H), |
| | hexyl]-methyl-amine | | | 4.35-4.27(m, 1H), |
| | | | | 3.47(AB_{q}, 2H), |
| | | | | 2.51-2.34(m, 2H), 2.21(s, |
| | | | | 3H), 1.87-1.72(m, |
| | | | | 2H), 1.64-1.24(m, |
| | | | | 4H), 0.90(dd, 3H) |
| 98 | (S)-Benzyl-[3-(3,4,5- | (R) | 402 | (CDCl₃) δ |
| | trichloro-phenoxy)- | | | 7.32-7.20(m, 5H), 6.98(s, 2H), |
| | hexyl]-methyl-amine | | | 4.35-4.27(m, 1H), |
| | | | | 3.47(AB_{q}, 2H), |
| | | | | 2.51-2.34(m, 2H), 2.21(s, |
| | | | | 3H), 1.87-1.72(m, |
| | | | | 2H), 1.64-1.24(m, |
| | | | | 4H), 0.90(dd, 3H) |

### Preparation 99

### Benzyl-[3-(2,4-dichloro-phenoxy)-4-methyl-pentyl]-methyl amine

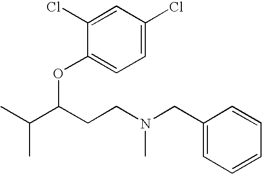

Add NaH (60% in mineral oil, 0.132 g, 3.32 mmol) to 1-(benzyl-methyl-amino)-4-methyl-3-ol (0.498 g, 2.21 mmol) in anhydrous DMSO (10 mL). After 30 minutes, add 1,3-dichloro-4-fluorobenzene (0.474 g, 2.87 mmol) and then heat the reaction mixture at 60° C. overnight. Cool the mixture and partition between EtOAc and water. Separate the layers and extract the aqueous layer with EtOAc. Combine the organic extracts, wash with aqueous saturated sodium chloride solution, dry over anhydrous Na₂SO₄, filter, and concentrate in vacuo. Purify the residue on silica gel eluting with 5% 2N NH₃ in MeOH/dichloromethane to give benzyl-[3-(2,4-dichloro-phenopxy)-4-methyl-pentyl]-methyl amine (0.385 g, 48%). Mass spectrum (ion spray): m/z=366 (M+1), ¹H NMR (CD₃OD) δ 7.34 (d, 1H), 7.28-7.17 (m, 6H), 7.06 (d, 1H), 4.35-4.31 (m, 1H), 3.48 (dd, 2H), 2.55-2.43 (m, 2H), 2.19 (s, 3H), 1.98-1.83 (m, 3H), 0.98 (dd, 6H).

A method similar to that described in Preparation 99 is used to prepare the following compounds:

| | | Mass | |
|---|---|---|---|
| | | Spectrum | |
| | | (ion spray) | |
| Preparation | Name | m/z (M+1) | ¹H NMR |
| 100 | Benzyl-[3-(2,4-dichloro- | 394 | (CD₃OD) δ 7.35(d, 1H), |
| | phenoxy)-5,5-dimethyl-hexyl]- | | 7.31-7.23(m, 5H), 7.18(d, 1H), |
| | methyl-amine | | 7.12(d, 1H), 4.62-4.57(m, |
| | | | 1H), 3.49(s, 2H), |
| | | | 2.50-2.44(m, 2H), 2.18(s, 3H), |
| | | | 1.86-1.73(m, 3H), 1.48(d, 1H), |
| | | | 0.92(s, 9H) |
| 101 | Benzyl-[3-cyclopropyl-3-(2,4- | 378 | (CD₃OD) δ 7.34(d, 1H), |
| | dichloro-phenoxy)-propyl]- | | 7.27-7.18(m, 6H), 7.07(d, 1H), |
| | methyl-amine | | 4.58-4.52(m, 1H), 3.48(dd, |
| | | | 2H), 2.58-2.46(m, 2H), |
| | | | 2.20(s, 3H), 2.01-1.91(m, 2H), |
| | | | 1.67-1.60(m, 1H), |
| | | | 1.49-1.42(m, 1H), 0.80-0.72(m, 1H), |
| | | | 0.47-0.40(m, 2H), |
| | | | 0.13-0.01(m, 2H) |
| 102 | (R)-Benzyl-[3-(2,4-dichloro- | 410 | (CDCl₃) δ 7.31(dd, 1H), |
| | phenoxy)-4-isobutoxy-butyl]- | | 7.28-7.18(m, 5H), 7.12(d, 2H), |
| | methyl-amine | | 4.57-4.50(m, 1H), |
| | | | 3.62-3.41(m, 4H), 3.24-3.15(m, 2H), |
| | | | 2.66-2.58(m, 1H), |
| | | | 2.49-2.41(m, 1H), 2.15(s, 3H), |
| | | | 1.94-1.75(m, 3H), 0.85(dd, 6H) |
| 103 | (R)-Benzyl-[3-(2,4-dichloro- | 396 | (CDCl₃) δ 7.31(d, 1H), |
| | phenoxy)-4-isopropoxy-butyl]- | | 7.28-7.19(m, 5H), 7.12(d, 2H), |
| | methyl-amine | | 4.53-4.46(m, 1H), |
| | | | 3.60-3.40(m, 5H), 2.66-2.57(m, 1H), |
| | | | 2.50-2.43(m, 1H), 2.15(s, |
| | | | 3H), 1.95-1.87(m, 2H), |
| | | | 1.10(dd, 6H) |
| 104 | (R)-Benzyl-[3-(2,4-dichloro- | 412 | N/A |
| | phenoxy)-4-isopropylsulfanyl- | | |
| | butyl]-methyl-amine | | |

### Preparation 105

### (R)-Benzyl-[4-tert-butoxy-3-(2,4-dichloro-phenoxy)-butyl]-methyl amine

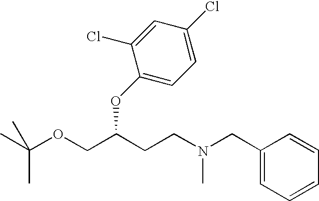

Add NaH (60% in mineral oil, 0.017 g, 0.413 mmol) to (R)-4-(benzyl-methyl-amino)-1-tert-butoxy-butan-2-ol (0.073 g, 0.275 mmol) in anhydrous DMSO (2 mL). After 30 minutes, add 1,3-dichloro-4-fluorobenzene ( 0.059 g, 0.358 mmol) and stir overnight. Add 2 drops of water, and then load the reaction mixture onto an SCX column. Wash with methanol and recover the amine material by eluting with 2N NH₃ in methanol. Concentrate basic filtrate in vacuo. Purify the residue on silica gel eluting with 5% 2N NH₃ in methanol/dichloromethane to give (R)-benzyl-[4-tert-butoxy-3-(2,4-dichloro-phenoxy)-butyl]-methyl amine (0.080 g, 71%). Mass spectrum (ion spray): m/z=410 (M+1), ¹H NMR (CD₃OD) δ7.33 (d, 1H), 7.26-7.15 (m, 7H), 4.52-4.47 (m, 1H), 3.57-3.46 (m, 4H), 2.63-2.45 (m, 2H), 2.19 (s, 3H), 1.99-1.85 (m, 2H), 1.15 (s, 9H).

A method similar to that described in Preparation 105 is used to prepare the following compounds:

| | | | Mass | |
|---|---|---|---|---|
| | | | Spectrum | |
| | | | (ion | |
| | | Reagent | spray) m/z | |
| Preparation | Name | Enantiomer | (M + 1) | ¹H NMR |
| 106 | (S)-Benzyl-[4-tert- | (S) | 410 | (CD₃OD) δ 7.33(d, 1H), |
| | butoxy-3-(2,4-dichloro- | | | 7.26-7.15(m, 7H), |
| | phenoxy)-butyl]-methyl | | | 4.52-4.47(m, 1H), 3.57-3.46(m, 4H), |
| | amine | | | 2.63-2.45(m, 2H), 2.19(s, |
| | | | | 3H), 1.99-1.85(m, 2H), |
| | | | | 1.15(s, 9H) |

### Preparation 107

### (R)-Toluene-4-sulfonic acid 3-hydroxy-hexyl ester and (S)-Toluene-4-sulfonic acid 3-hydroxy-hexyl ester

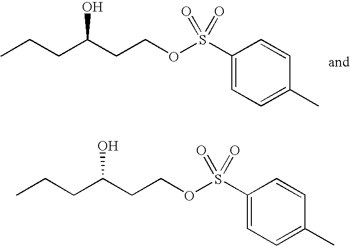

Separate the racemic mixture of toluene-4-sulfonic acid 3-hydroxy-hexyl ester into the two enantiomers by chiral chromatography on 4.6×250 mm Chiralcel AD eluting with 7.5% isopropanol/7.5% methanol/heptanes at flow 0.6 mL/min (uv: 260 nm)

Enantiomer 1 (11.04 g)/(first eluting enantiomer)=99.1% ee

Enantiomer 2 (11.40 g)/(second eluting enantiomer)=96.7% ee

Use of a standard sample from Preparation 17, which may be prepared from the diol in Preparation 3 of known absolute configuration, enantiomer 1 is identified as (R) stereochemistry and enantiomer 2 as (S) stereochemistry.

### Preparation 108

### (R)-Benzyl-methyl-(2-oxiranyl-ethyl)-amine and (S)-benzyl-methyl-(2-oxiranyl-ethyl)-amine

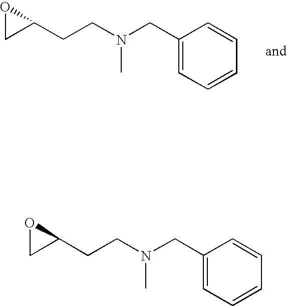

Separate the racemic mixture of benzyl-methyl-(2-oxiranyl-ethyl)-amine into its constitutive two enantiomers by chiral chromatography on 4.6×250 mm Chiralcel AD eluting with 2% isopropanol/1% methanol/heptanes at flow 1.0 mL/min (uv: 250 nm)

(R) Enantiomer (10.52 g)/(first eluting enantiomer)=98% ee

(S) Enantiomer (9.67 g)/(second eluting enantiomer)=98.1% ee

Use of a standard sample from Preparation 40, enantiomer 1 was identified as (R) stereochemistry and enantiomer 2 as (S) stereochemistry.

### Preparation 109

### 1-(Benzyl-methyl-amino)-6,6,6-trifluoro-hexan-3-ol -enantiomer 1 and 1-(benzyl-methyl-amino)-6,6,6-trifluoro-hexan-3-ol-enantiomer 2.

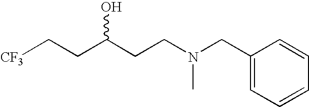

Separate the racemic mixture of 1-(benzyl-methyl-amino)-6,6,6-trifluoro-hexan-3-ol into its constitutive two enantiomers by chiral chromatography on 4.6×250 mm Chiralcel OJ eluting with 3% isopropanol in heptanes with 0.2% DMEA at flow 1.0 mL/min (uv: 250 nm)

Enantiomer 1 (0.208 g)/(first eluting enantiomer)=100% ee

Enantiomer 2 (0.200 g)/(second eluting enantiomer)=99.4% ee

### Preparation 110

### 1-(Benzyl-methyl-amino)-6-methyl-heptan-3-ol -enantiomer 1 and 1-(benzyl-methyl-amino)-6-methyl-heptan-3-ol -enantiomer 2.

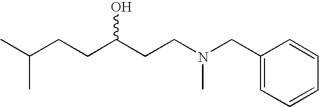

Separate the racemic mixture of 1-(benzyl-methyl-amino)-6-methyl-heptan-3-ol into its constitutive two enantiomers by chiral chromatography on 4.6×150 mm Chiralcel OJ eluting with 1% isopropanol/hexanes with 0.2% DMEA at flow 1.0 mL/min (uv: 260 nm)

Enantiomer 1 (0.527 g)/(first eluting enantiomer)=99.5% ee

Enantiomer 2 (0.492 g)/(second eluting enantiomer)=99.6% ee

### Preparation 111

### Benzyl-[3-(2,4-dichloro-phenoxy)-butyl]-methyl-amine-enantiomer 1 and benzyl-[3-(2,4-dichloro-phenoxy)-butyl]-methyl-amine-enantiomer 2.

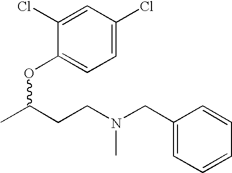

Separate the racemic mixture of benzyl-[3-(2,4-dichloro-phenoxy)-butyl]-methyl-amine into its constitutive two enantiomers by chiral chromatography on 4.6×150 mm Chiralcel OJ eluting with 100% methanol at flow 0.6 mL/min (uv: 280 nm)

Enantiomer 1 (0.401 g)/(first eluting enantiomer)=100% ee

Enantiomer 2 (0.422 g)/(second eluting enantiomer)=99.7% ee

### Preparation 112

### Benzyl-[3-(2,4-dichloro-phenoxy)-pentyl]-methyl-amine-enantiomer 1 and benzyl-[3-(2,4-dichloro-phenoxy)-phenyl]-methyl-amine-enantiomer 2.

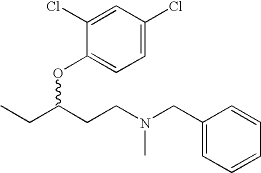

Separate the racemic mixture of benzyl-[3-(2,4-dichloro-phenoxy)-pentyl]-methyl-amine into its constitutive two enantiomers by chiral chromatography on 4.6×150 mm Chiralcel OJ eluting with 100% methanol at flow 0.6 mL/min (uv: 230 nm)

Enantiomer 1 (0.385 g)/(first eluting enantiomer)=100% ee

Enantiomer 2 (0.374 g)/(second eluting enantiomer)=99.4% ee

### Preparation 113

### Benzyl-[3-(2,4-dichloro-phenoxy)-4-methylpentyl]-methyl-amine-enantiomer 1 and benzyl-[3-(2,4-dichloro-phenoxy)-4-methylpentyl]-methyl-amine-enantiomer 2.

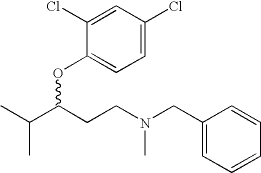

Separate the racemic mixture of benzyl-[3-(2,4-dichloro-phenoxy)-4-methyl-pentyl]-methyl amine into its constitutive two enantiomers by chiral chromatography on 4.6×150 mm Chiralcel OJ eluting with 0.2% DMEA in methanol at flow 0.6 mL/min (uv: 230 nm)

Enantiomer 1 (0.167 g)/(first eluting enantiomer)=100% ee

Enantiomer 2 (0.168 g)/(second eluting enantiomer)=99.4% ee

### EXAMPLES

### Example 1

### (S)-Methyl-[3-(2-trifluoromethyl-phenoxy)-hexyl]-amine

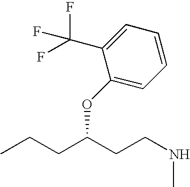

Add 1-chloroethyl chloroformate (0.281 mL, 2.6 mmol) to a solution of (S)-benzyl-methyl-[3-(2-trifluoromethyl-phenoxy)-hexyl]-amine (0.41 g, 1.1 mmol) in anhydrous 1,2-dichloroethane (13 mL). Heat the reaction mixture at reflux for 90 minutes, then cool the reaction briefly and add methanol (16 mL). Heat the reaction mixture at reflux for 45 minutes, then cool and make the reaction basic by the addition of 2N NH₃ in methanol. Concentrate and purify on silica gel eluting with 1% NH₄OH/10% ethanol/chloroform to give (S)-methyl-[3-(2-trifluoromethyl-phenoxy)-hexyl]-amine (0.230 g, 74%). Mass spectrum (ion spray): m/z=276 (M+1), ¹H NMR (CDCl₃) δ 7.53 (d, 1H), 7.44 (dd, 1H), 7.02 (d, 1H), 6.95 (dd, 1H), 4.58-4.55 (m, 1H), 2.85 (brs, 2H), 2.50 (brs, 3H), 2.07-1.99 (m, 2H), 1.76-1.59 (m, 2H), 1.45-1.38 (m, 2H), 0.92 (t, 3H).

A method similar to that described in Example 1 is used to prepare the following compounds:

| | | Mass Spectrum | |
|---|---|---|---|
| | | (ion spray) m/z | |
| Example | Name | (M + 1) | ¹H NMR |
| 2 | (S)-[3-(3-Chloro-phenoxy)- | 242 | (CD₃OD) δ 7.22(dd, 1H), |
| | hexyl]-methyl-amine | | 6.94-6.83(m, 3H), |
| | | | 4.46-4.40(m, 1H), 2.72-2.60(m, 2H), |
| | | | 2.36(s, 3H), 1.90-1.83(m, |
| | | | 2H), 1.73-1.56(m, 2H), |
| | | | 1.51-1.35(m, 2H), 0.94(t, 3H) |
| 3 | (S)-[3-(3-Chloro-4-fluoro- | 260 | (CD₃OD) δ 7.13(dd, 1H), |
| | phenoxy)-hexyl]-methyl- | | 7.06-7.03(m, 1H), |
| | amine | | 6.91-6.86(m, 1H), 4.43-4.37(m, 1H), |
| | | | 2.89-2.80(m, 2H), 2.50(s, |
| | | | 3H), 1.97-1.88(m, 2H), |
| | | | 1.72-1.55(m, 2H), 1.50-1.36(m, |
| | | | 2H), 0.94(t, 3H) |
| 4 | (S)-[3-(4-Chloro-3- | 310 | (CD₃OD) δ 7.48(d, 1H), |
| | trifluoromethyl-phenoxy)- | | 7.27(d, 1H), 7.17(dd, 1H), |
| | hexyl]-methyl-amine | | 4.55-4.49(m, 1H), 2.87-2.75(m, |
| | | | 2H), 2.47(s, 3H), |
| | | | 1.97-1.90(m, 2H), 1.73-1.59(m, 2H), |
| | | | 1.50-1.38(m, 2H), 0.95(t, |
| | | | 3H) |
| 5 | (S)-[3-(2-Chloro-4-fluoro- | 260 | (CD₃OD) δ 7.16(dd, 1H), |
| | phenoxy)-hexyl]-methyl- | | 7.07(dd, 1H), 7.02-6.97(m, |
| | amine | | 1H), 4.47-4.40(m, 1H), |
| | | | 2.71-2.63(m, 2H), 2.35(s, 3H), |
| | | | 1.92-1.86(m, 2H), |
| | | | 1.74-1.56(m, 2H), 1.51-1.38(m, 2H), |
| | | | 0.94(t, 3H) |
| 6 | (S)-[3-(2-Chloro-4- | 310 | (CD₃OD) δ 7.64(d, 1H), |
| | trifluoromethyl-phenoxy)- | | 7.54(dd, 1H), 7.24(d, 1H), |
| | hexyl]-methyl-amine | | 4.68-4.61(m, 1H), 2.73-2.62(m, |
| | | | 2H), 2.36(s, 3H), |
| | | | 1.97-1.90(m, 2H), 1.72-1.62(m, 2H), |
| | | | 1.53-1.38(m, 2H), 0.95(t, |
| | | | 3H) |
| 7 | (S)-[3-(4-Fluoro-naphthalen- | 276 | (CD₃OD) δ 8.21(d, 1H), |
| | 1-yloxy)-hexyl]-methyl- | | 7.97(d, 1H), 7.57-7.49(m, 2H), |
| | amine | | 7.08-7.02(m, 1H), 6.84(dd, |
| | | | 1H), 4.62-4.56(m, 1H), |
| | | | 2.77-2.62(m, 2H), 2.32(s, 3H), |
| | | | 2.03-1.92(m, 2H), |
| | | | 1.84-1.66(m, 2H), 1.57-1.40(m, 2H), |
| | | | 0.94(t, 3H) |
| 8 | (S)-[3-(2,3-Difluoro-4- | 258 | (CD₃OD) δ 6.88(dd, 1H), |
| | methyl-phenoxy)-hexyl]- | | 6.79(dd, 1H), 4.42-4.34(m, |
| | methyl-amine | | 1H), 2.73-2.62(m, 2H), |
| | | | 2.36(s, 3H), 2.22(s, 3H), |
| | | | 1.90-1.82(m, 2H), 1.73-1.56(m, |
| | | | 2H), 1.50-1.39(m, 2H), |
| | | | 0.94(t, 3H) |
| 9 | (R)-[3-(4-Fluoro-naphthalen- | 276 | (CD₃OD) δ 8.22(d, 1H), |
| | 1-yloxy)-hexyl]-methyl- | | 7.98(d, 1H), 7.57-7.49(m, 2H), |
| | amine | | 7.05(dd, 1H), 6.85(dd, 1H), |
| | | | 4.63-4.58(m, 1H), |
| | | | 2.77-2.63(m, 2H), 2.33(s, 3H), |
| | | | 2.02-1.94(m, 2H), 1.84-1.67(m, |
| | | | 2H), 1.56-1.43(m, 2H), |
| | | | 0.94(t, 3H) |
| 10 | [3-(2,4-Dichloro-phenoxy)- | 330 | (CD₃OD) δ 7.41(d, 1H), |
| | 6,6,6-trifluoro-hexyl]-methyl- | | 7.26(dd, 1H), 7.12(d, 1H), |
| | amine Enantiomer 1 | | 4.62-4.56(m, 1H), 2.71-2.62(m, |
| | | | 2H), 2.39-2.23(m, 5H), |
| | | | 2.00-1.84(m, 4H) |
| 11 | [3-(2,4-Dichloro-phenoxy)-4- | 276 | (CD₃OD) δ 7.37(d, 1H), |
| | methyl-penty]-methyl-amine | | 7.22(dd, 1H), 7.08(d, 1H), |
| | Enantiomer 1 | | 4.37-4.31(m, 1H), 2.72-2.58(m, |
| | | | 2H), 2.35(s, 3H), |
| | | | 2.03-1.80(m, 3H), 0.99(dd, 6H) |
| 12 | [3-(2,4-Dichloro- | 304 | (CD₃OD) δ 7.34(d, 1H), |
| | phenoxy)-5,5-dimethyl- | | 7.24(dd, 1H), 7.12(d, 1H), |
| | hexyl]-methyl-amine | | 4.65-4.62(m, 1H), |
| | | | 2.64(dd, 2H), 2.34(s, 3H), |
| | | | 1.92-1.77(m, 3H), |
| | | | 1.54(dd, 1H), 0.95(s, 9H) |
| 13 | [4-Cyclopropyl-3-(2,4- | 288 | (CD₃OD) δ 7.40(d, 1H), |
| | dichloro-phenoxy)-butyl]- | | 7.27-7.24(m, 1H), 7.14(d, |
| | methyl-amine | | 1H), 4.66-4.59(m, 1H), |
| | | | 3.03-2.91(m, 2H), 2.56(s, 3H), |
| | | | 2.15-2.07(m, 2H), |
| | | | 1.71-1.50(m, 2H), 0.82-0.73(m, 1H), |
| | | | 0.51-0.43(m, 2H), |
| | | | 0.17-0.06(m, 2H) |
| 14 | (S)-Methyl-[3-(4- | 276 | (CDCl₃) δ 7.51(d, 2H), |
| | trifluoromethyl-phenoxy)- | | 6.96(d, 2H), 4.47-4.44(m, 1H), |
| | hexyl]-amine | | 2.72-2.68(m, 2H), 2.42(s, |
| | | | 3H), 1.90-1.80(m, 2H), |
| | | | 1.71-1.58(m, 2H), 1.47-1.36(m, |
| | | | 2H), 0.93(t, 3H) |
| 15 | (S)-[3-(2-Chloro-phenoxy)- | 243 | (CD₃OD) δ 7.33(1H), |
| | hexyl]-methyl-amine | | 7.21(dd, 1H), 7.06(d, 1H), |
| | | | 6.87(dd, 1H), 4.51-4.48(m, 1H), |
| | | | 2.71-2.67(m, 2H), 2.35(s, |
| | | | 3H), 1.92-1.88(m, 2H), |
| | | | 1.74-1.60(m, 2H), 1.49-1.40(m, |
| | | | 2H), 0.94(t, 3H) |
| 16 | (S)-[3-(4-Chloro-phenoxy)- | 243 | (CD₃OD) δ 7.22(d, 2H), |
| | hexyl]-methyl-amine | | 6.88(d, 2H), 4.87-4.36(m, 1H), |
| | | | 2.68-2.61(m, 2H), 2.35(s, |
| | | | 3H), 1.87-1.82(m, 2H), |
| | | | 1.67-1.56(m, 2H), |
| | | | 1.47-1.38(m, 2H), 0.94(t, 3H) |
| 17 | (S)-[3-(2,3-Dichloro- | 276 | (CDCl₃) δ 7.09(dd, 1H), |
| | phenoxy)-hexyl]-methyl- | | 7.02(d, 1H), 6.88(d, 1H), |
| | amine | | 4.46-4.40(m, 1H), 2.72-2.69(m, |
| | | | 2H), 2.42(s, 3H), |
| | | | 1.94-1.82(m, 2H), 1.78-1.58(m, 2H), |
| | | | 1.49-1.37(m, 2H), 0.93(t, |
| | | | 3H) |
| 18 | (S)-[3-(Naphthalen-2-yloxy)- | 258 | (CDCl₃) δ 7.76-7.69(m, 3H), |
| | hexyl]-methyl-amine | | 7.42(dd, 1H), 7.31(dd, 1H), |
| | | | 7.18(s, 1H), 7.14(d, 1H), |
| | | | 4.56-4.51(m, 1H), 2.75(brs, |
| | | | 2H), 2.44(brs, 3H), |
| | | | 1.97-1.89(m, 2H), 1.85-1.62(m, 2H), |
| | | | 1.54-1.41(m, 2H), 0.96(t, |
| | | | 3H) |
| 19 | (S)-[3-(naphthalen-1-yloxy)- | 257 GC-Mass | (CDCl₃) δ 8.28(d, 1H), |
| | hexyl]-methyl-amine | | 7.78(d, 1H), 7.50-7.34(m, 4H), |
| | | | 6.86(d, 1H), 4.62-4.59(m, |
| | | | 1H), 2.76(brs, 2H), 2.41(brs, |
| | | | 3H), 2.04-1.94(m, 2H), |
| | | | 1.86-1.68(m, 2H), 1.57-1.43(m, |
| | | | 2H), 0.94(t, 3H) |
| 20 | (S)-[3-(2-Chloro-3- | 310 | (CD₃OD) δ 7.43-7.30(m, |
| | trifluoromethyl-phenoxy)- | | 3H), 4.64-4.58(m, 1H), |
| | hexyl]-methyl-amine | | 2.75-2.70(m, 2H), 2.38(s, 3H), |
| | | | 1.97-1.92(m, 2H), |
| | | | 1.75-1.66(m, 2H), 1.51-1.44(m, 2H), |
| | | | 0.96(t, 3H) |
| 21 | (S)-[3-(2,3,5-Trichloro- | 311 | (CD₃OD) δ 7.16(s, 1H), |
| | phenoxy)-hexyl]-methyl- | | 7.14(s, 1H), 4.59-4.56(m, 1H), |
| | amine | | 2.70-2.60(m, 2H), 2.36(s, |
| | | | 3H), 1.93-1.88(m, 2H), |
| | | | 1.70-1.63(m, 2H), 1.46-1.40(m, |
| | | | 2H), 0.94(t, 3H) |
| 22 | (R)-[3-(4-Chloro-phenoxy)- | 243 | (CD₃OD) δ 7.22(d, 2H), |
| | hexyl]-methyl-amine | | 6.88(d, 2H), 4.87-4.36(m, 1H), |
| | | | 2.68-2.61(m, 2H), 2.35(s, |
| | | | 3H), 1.87-1.82(m, 2H), |
| | | | 1.67-1.56(m, 2H), |
| | | | 1.47-1.38(m, 2H), 0.94(t, 3H) |
| 23 | (R)-[3-(2,3-Dichloro- | 276 | (CDCl₃) δ 7.09(dd, 1H), |
| | phenoxy)-hexyl]-methyl- | | 7.02(d, 1H), 6.88(d, 1H), |
| | amine | | 4.46-4.40(m, 1H), 2.72-2.69(m, |
| | | | 2H), 2.42(s, 3H), |
| | | | 1.94-1.82(m, 2H), 1.78-1.58(m, 2H), |
| | | | 1.49-1.37(m, 2H), 0.93(t, |
| | | | 3H) |
| 24 | (R)-[3-(Naphthalen-2-yloxy)- | 258 | (CDCl₃) δ 7.76-7.69(m, 3H), |
| | hexyl]-methyl-amine | | 7.42(dd, 1H), 7.31(dd, 1H), |
| | | | 7.18(s, 1H), 7.14(d, 1H), |
| | | | 4.56-4.51(m, 1H), 2.75(brm, |
| | | | 2H), 2.44(brs, 3H), |
| | | | 1.97-1.89(m, 2H), 1.85-1.62(m, 2H), |
| | | | 1.54-1.41(m, 2H), 0.96(t, |
| | | | 3H) |
| 25 | (R)-[3-(Naphthalen-1-yloxy)- | 257 GC-Mass | (CDCl₃) δ 8.28(d, 1H), |
| | hexyl]-methyl-amine | | 7.78(d, 1H), 7.50-7.34(m, 4H), |
| | | | 6.86(d, 1H), 4.62-4.59(m, |
| | | | 1H), 2.76(brs, 2H), 2.41(brs, |
| | | | 3H), 2.04-1.94(m, 2H), |
| | | | 1.86-1.68(m, 2H), 1.57-1.43(m, |
| | | | 2H), 0.94(t, 3H) |
| 26 | (R)-[3-(2-Chloro-3- | 310 | (CD₃OD) δ 7.43-7.30(m, |
| | trifluoromethyl-phenoxy)- | | 3H), 4.64-4.58(m, 1H), |
| | hexyl]-methyl-amine | | 2.75-2.7(m, 2H), 2.38(s, 3H), |
| | | | 1.97-1.92(m, 2H), |
| | | | 1.75-1.66(m, 2H), 1.51-1.44(m, 2H), |
| | | | 0.96(t, 3H) |
| 27 | (R)-[3-(2,3,5-Trichloro- | 311 | (CD₃OD) δ 7.16(s, 1H), |
| | phenoxy)-hexyl]-methyl- | | 7.14(s, 1H), 4.59-4.56(m, 1H), |
| | amine | | 2.70-2.60(m, 2H), 2.36(s, |
| | | | 3H), 1.93-1.88(m, 2H), |
| | | | 1.70-1.63(m, 2H), 1.46-1.40(m, |
| | | | 2H), 0.94(t, 3H) |
| 28 | 3-(2,4-Dichloro-phenoxy)- | 249 | (CD₃OD) δ 7.38(d, 1H), |
| | butyl]-methyl-amine | | 7.24-7.21(m, 1H), 7.06(d, |
| | Enantiomer 1 | | 1H), 4.59-4.55(m, 1H), |
| | | | 2.74-2.69(m, 2H), 2.37(s, 3H), |
| | | | 1.98-1.86(m, 2H), 1.31(d, |
| | | | 3H) |
| 29 | [3-(2,4-Dichloro-phenoxy)- | 263 | (CD₃OD) δ 7.38(d, 1H), |
| | pentyl]-methyl-amine | | 7.24-7.21(m, 1H), 7.07(d, |
| | Enantiomer 1 | | 1H), 4.46-4.41(m, 1H), |
| | | | 2.38(s, 3H), 1.94-1.89(m, 2H), |
| | | | 1.75-1.69(m, 2H), 0.98(t, |
| | | | 3H) |
| 30 | (S)-[3-(2,4-Dichloro- | 276 | (CDCl₃) δ 7.34(d, 1H), |
| | phenoxy)-hexyl]-methyl- | | 7.14(dd, 1H), 6.92(d, 1H), |
| | amine | | 4.41-4.35(m, 1H), 2.71-2.66(m, |
| | | | 2H), 2.41(s, 3H), |
| | | | 1.93-1.56(m, 4H), 1.48-1.35(m, 2H), |
| | | | 1.11(brs, 1H), 0.91(t, 3H) |
| 31 | (S)-[3-(3,4-Dichloro- | 276 | (CDCl₃) δ 7.28(dd, 1H), |
| | phenoxy)-hexyl]-methyl- | | 7.03(d, 1H), 6.76(dd, 1H), |
| | amine | | 4.35-4.29(m, 1H), 2.70-2.60(m, |
| | | | 2H), 2.41(s, 3H), |
| | | | 1.84-1.75(m, 2H), 1.69-1.52(m, 2H), |
| | | | 1.47-1.31(m, 2H), 1.10(brs, |
| | | | 1H), 0.91(t, 3H) |
| 32 | (R)-[3-(3,4-Dichloro- | 276 | (CDCl₃) δ 7.28(dd, 1H), |
| | phenoxy)-hexyl]-methyl- | | 7.03(d, 1H), 6.76(dd, 1H), |
| | amine | | 4.35-4.29(m, 1H), 2.70-2.60(m, |
| | | | 2H), 2.41(s, 3H), |
| | | | 1.84-1.75(m, 2H), 1.69-1.52(m, 2H), |
| | | | 1.47-1.31(m, 2H), 1.10(brs, |
| | | | 1H), 0.91(t, 3H) |
| 33 | (S)-[3-(3,5-Dichloro- | 276 | (CDCl₃) δ 6.91(dd, 1H), |
| | phenoxy)-hexyl]-methyl- | | 6.82(d, 2H), 4.37-4.31(m, 1H), |
| | amine | | 2.68-2.60(m, 2H), 2.42(s, |
| | | | 3H), 1.86-1.74(m, 2H), |
| | | | 1.70-1.53(m, 2H), 1.47-1.30(m, |
| | | | 2H), 1.04(brs, 1H), 0.92(t, |
| | | | 3H) |
| 34 | (R)-[3-(3,5-Dichloro- | 276 | (CDCl₃) δ 6.91(dd, 1H), |
| | phenoxy)-hexyl]-methyl- | | 6.82(d, 2H), 4.37-4.31(m, 1H), |
| | amine | | 2.68-2.60(m, 2H), 2.42(s, |
| | | | 3H), 1.86-1.74(m, 2H), |
| | | | 1.70-1.53(m, 2H), 1.47-1.30(m, |
| | | | 2H), 1.04(brs, 1H), 0.92(t, |
| | | | 3H) |
| 35 | (S)-[3-(2,4-Dichloro-6- | 290 | (CDCl₃) δ 7.19(d, 1H), |
| | methyl-phenoxy)-hexyl]- | | 7.04(d, 1H), 4.41-4.37(m, 1H), |
| | methyl-amine | | 2.78-2.69(m, 2H), 2.43(s, |
| | | | 3H), 2.25(s, 3H), |
| | | | 1.90-1.77(m, 2H), 1.60-1.27(m, 5H), |
| | | | 0.89(t, 3H) |
| 36 | (S)-[3-(4-Chloro-3,5- | 270 | (CDCl₃) δ 6.65(s, 2H), |
| | dimethyl-phenoxy)-hexyl]- | | 4.32-4.27(m, 1H), 2.70-2.63(m, |
| | methyl-amine | | 2H), 2.41(s, 3H), 2.32(s, |
| | | | 6H), 1.84-1.76(m, 2H), |
| | | | 1.68-1.51(m, 2H), 1.48-1.32(m, |
| | | | 2H), 1.04(brs, 1H), 0.91(t, |
| | | | 3H) |
| 37 | (R)-[3-(4-Chloro-3,5- | 270 | (CDCl₃) δ 6.65(s, 2H), |
| | dimethyl-phenoxy)-hexyl]- | | 4.32-4.27(m, 1H), 2.70-2.63(m, |
| | methyl-amine | | 2H), 2.41(s, 3H), 2.32(s, |
| | | | 6H), 1.84-1.76(m, 2H), |
| | | | 1.68-1.51(m, 2H), 1.48-1.32(m, |
| | | | 2H), 1.04(brs, 1H), 0.91(t, |
| | | | 3H) |
| 38 | [3-(2,4-Dichloro-phenoxy)-6- | 304 | (CDCl₃) δ 7.34(d, 1H), |
| | methyl-heptyl]-methyl-amine | | 7.14(dd, 1H), 6.90(d, 1H), |
| | Enantiomer 2 | | 4.37-4.31(m, 1H), 2.73-2.65(m, |
| | | | 2H), 2.41(s, 3H), |
| | | | 1.93-1.78(m, 2H), 1.75-1.57(m, 2H), |
| | | | 1.55-1.46(m, 1H), 1.39(brs, |
| | | | 1H), 1.34-1.18(m, 2H), |
| | | | 0.86(dd, 6H) |
| 39 | (R)-[3-(2,4-Dichloro- | 278 | (CDCl₃) δ 7.34(d, 1H), |
| | phenoxy)-4-methoxy-butyl]- | | 7.16(dd, 1H), 7.06(d, 1H), |
| | methyl-amine | | 4.52-4.44(m, 1H), 3.62-3.50(m |
| | | | 2H), 3.37(s, 3H), |
| | | | 2.78-2.67(m, 2H), 2.40(s, 3H), |
| | | | 1.95-1.83(m, 2H), 1.25(brs, 1H) |
| 40 | (R)-[3-(2,4-Dichloro- | 292 | (CDCl₃) δ 7.33(d, 1H), |
| | phenoxy)-4-ethoxy-butyl]- | | 7.15(dd, 1H), 7.07(d, 1H), |
| | methyl-amine | | 4.52-4.45(m, 1H), 3.64-3.46(m, |
| | | | 4H), 2.77-2.68(brm, 2H), |
| | | | 2.41(s, 3H), 1.94-1.86(m, |
| | | | 2H), 1.34(brs, 1H), 1.15(t, |
| | | | 3H) |
| 41 | [3-(2,3-Dichloro-phenoxy)-6- | 304 | (CDCl₃) δ 7.07(dd, 1H), |
| | methyl-heptyl]-methyl-amine | | 6.93(dd, 2H), 4.39-4.33(m, 1H), |
| | Enantiomer 1 | | 2.70-2.64(m, 2H), 2.38(s, |
| | | | 3H), 1.92-1.77(m, 2H), |
| | | | 1.74-1.57(m, 2H), 1.54-1.43(m, |
| | | | 1H), 1.36(brs, 1H), |
| | | | 1.33-1.15(m, 2H), 0.84(dd, 6H) |
| 42 | [3-(2,3-Dichloro-phenoxy)-6- | 304 | (CDCl₃) δ 7.07(dd, 1H), |
| | methyl-heptyl]-methyl-amine | | 6.93(dd, 2H), 4.39-4.33(m, 1H), |
| | Enantiomer 2 | | 2.70-2.64(m, 2H), 2.38(s, |
| | | | 3H), 1.92-1.77(m, 2H), |
| | | | 1.74-1.57(m, 2H), 1.54-1.43(m, |
| | | | 1H), 1.33-1.15(m, 3H), |
| | | | 0.84(dd, 6H) |
| 43 | (S)-[3-(4-Chloro-2- | 310 | (CDCl₃) δ 7.52(d, 1H), |
| | trifluoromethyl-phenoxy)- | | 7.39(dd, 1H), 6.99(d, 1H), |
| | hexyl]-methyl-amine | | 4.54-4.46(m, 1H), 2.67(brs, 2H), |
| | | | 2.40(brs, 3H), 1.90-1.80(m, |
| | | | 2H), 1.76-1.56(m, 2H), |
| | | | 1.49-1.32(m, 2H), 1.23(brs, 1H), |
| | | | 0.91(t, 3H) |
| 44 | (R)-[3-(2,4-Dichloro- | 320 | (CDCl₃) δ 7.33(d, 1H), |
| | phenoxy)-4-isobutoxy-butyl]- | | 7.14(dd, 1H), 7.09(d, 1H), |
| | methyl-amine | | 4.53-4.47(m, 1H), 3.64-3.52(m, |
| | | | 2H), 3.24-3.16(m, 2H), |
| | | | 2.78-2.70(brm, 2H), 2.42(brs, |
| | | | 3H), 1.95-1.88(m, 2H), |
| | | | 1.84-1.76(m, 1H), 1.51(brs, 1H), |
| | | | 0.85(dd, 6H) |
| 45 | (R)-[3-(2,4-Dichloro- | 306 | (CDCl₃) δ 7.33(d, 1H), |
| | phenoxy)-4-isopropoxy- | | 7.14(dd, 1H), 7.08(d, 1H), |
| | butyl]-methyl-amine | | 4.49-4.42(m, 1H), 3.63-3.52(m, |
| | | | 3H), 2.74(brs, 2H), 2.41(brs, |
| | | | 3H), 1.95-1.89(m, 2H), |
| | | | 1.42(brs, 1H), 1.11(dd, 6H) |
| 46 | (R)-[3-(2,4-Dichloro- | 322 | (CDCl₃) δ 7.35(d, 1H), |
| | phenoxy)-4- | | 7.16(dd, 1H), 6.99(d, 1H), |
| | isopropylsylfanyl-butyl]- | | 4.55-4.46(m, 1H), 3.02-2.93(m, |
| | methyl-amine | | 1H), 2.89-2.82(m, 1H), |
| | | | 2.80-2.71(m, 3H), 2.44(s, 3H), |
| | | | 2.08-2.12(m, 2H), 1.47(brs, |
| | | | 1H), 1.25(dd, 6H) |
| 47 | (R)-[4-tert-Butoxy-3-(2,4- | 320 | (CD₃OD) δ 7.34(d, 1H), |
| | dichloro-phenoxy)-butyl]- | | 2.27-7.21(m, 2H), |
| | methyl amine | | 4.64-4.58(m, 1H), 3.57(d, 2H), |
| | | | 3.21-3.14(m, 2H), 2.68(s, 3H), |
| | | | 2.20-2.14(m, 2H), 1.45(s, |
| | | | 9H) |
| 48 | (S)-[4-tert-butoxy-3-(2,4- | 320 | (CD₃OD) δ 7.34(d, 1H), |
| | dichloro-phenoxy)-butyl]- | | 7.27-7.21(m, 2H), |
| | methyl-amine | | 4.64-4.58(m, 1H), 3.57(d, 2H), |
| | | | 3.21-3.14(m, 2H), 2.68(s, 3H), |
| | | | 2.20-2.14(m, 2H), 1.45(s, |
| | | | 9H) |
| 49 | (R)-[3-(2,3,4-trichloro- | N/A | (CDCl₃) δ 7.41(d, 1H), |
| | phenoxy)-hexyl]-methyl- | | 7.08(d, 1H), 4.58-4.50(m, 1H), |
| | amine | | 2.72-2.60(m, 2H), 2.35(s, |
| | | | 3H), 1.95-1.86(m, 2H), |
| | | | 1.76-1.59(m, 2H), 1.52-1.33(m, |
| | | | 2H), 0.94(dd, 3H) |
| 50 | (S)-[3-(2,3,4-trichloro- | N/A | (CDCl₃) δ 7.41(d, 1H), |
| | phenoxy)-hexyl]-methyl- | | 7.08(d, 1H), 4.58-4.50(m, 1H), |
| | amine | | 2.72-2.60(m, 2H), 2.35(s, |
| | | | 3H), 1.95-1.86(m, 2H), |
| | | | 1.76-1.59(m, 2H), 1.52-1.33(m, |
| | | | 2H), 0.94(dd, 3H) |
| 51 | (R)-[3-(3,4,5-trichloro- | 312 | (CDCl₃) δ 7.00(s, 2H), |
| | phenoxy)-hexyl]-methyl- | | 4.38-4.30(m, 1H), 2.71-2.57(m, |
| | amine | | 2H), 2.43(s, 3H), |
| | | | 1.90-1.72(m, 2H), 1.70-1.52(m, 2H), |
| | | | 1.50-1.30(m, 2H), 0.93(dd, |
| | | | 3H) |
| 52 | (S)-[3-(3,4,5-trichloro- | 312 | (CDCl₃) δ 7.00(s, 2H), |
| | phenoxy)-hexyl]-methyl- | | 4.38-4.30(m, 1H), 2.71-2.57(m, |
| | amine | | 2H), 2.43(s, 3H), |
| | | | 1.90-1.72(m, 2H), 1.70-1.52(m, 2H), |
| | | | 1.50-1.30(m, 2H), 0.93(dd, |
| | | | 3H) |

### Example 53

### (R)-[3-(2,4-Dichloro-phenoxy)-4-morpholin-4-yl-butyl]-methyl-amine

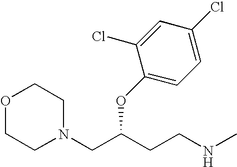

Add trifluoroacetic acid (6.0 mL, 72.88 mmol) to a cool (0° C.) solution of (R)-[3-(2,4-dichloro-phenoxy)-4-morpholin-4-yl-butyl]-methyl-carbamic acid tert-butyl ester (0.1741 g, 0.402 mmol), anisole (9.0 mL, 82.81 mmol) in dichloromethane (5 mL) and stir for 1.5 hours at 0° C. before allowing the reaction mixture to warm to ambient temperature and stirring for 1.5 hours. Pour the reaction mixture onto an SCX column. Wash with methanol, then elute the basic material with 2N NH₃ in methanol. Concentrate the basic methanol fractions to give (R)-[3-(2,4-dichloro-phenoxy)-4-morpholin-4-yl-butyl]-methyl-amine (0.1279 g, 95%). Mass spectrum (ion spray):

m/z=333 (M+1), ¹H NMR (CDCl₃) δ7.35 (d, 1H), 7.15 (dd, 1H), 7.04 (d, 1H), 4.57-4.50 (m, 1H), 3.65-3.58 (m, 4H), 2.76-2.65 (m, 3H), 2.59-2.45 (m, 5H), 2.43 (s, 3H), 1.97-1.82 (m, 3H).

### Example 54

### (R)-[3-(2,4-Dichloro-phenoxy)-4-pyrrolidin-1-yl-butyl]-methyl-amine

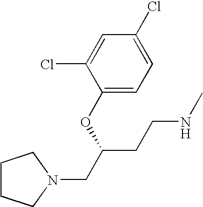

Using a method similar to that described in Example 53, using [3-(2,4-dichloro-phenoxy)-4-pyrrolidin-1-yl-butyl]-methyl-carbamic acid tert-butyl ester affords the title compound. Mass spectrum (m/z): m/z=317 (M+1), ¹H NMR (CDCl₃) δ7.34 (d, 1H), 7.14 (dd, 1H), 7.01 (d, 1H), 4.56-4.50 (m, 1H), 2.78-2.68 (m, 4H), 2.61-2.52 (m, 4H), 2.42 (s, 3H), 1.99-1.90 (m, 3H), 1.76-1.71 (m, 4H).

### Example 55

### (S)-[3-(3-Chloro-phenoxy)-hexyl]-methyl-amine hydrochloride

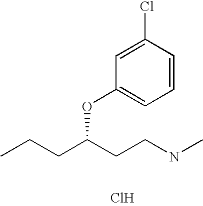

Add NH₄Cl (0.018 g, 0.328 mmol) to a solution of (S)-[3-(3-chloro-phenoxy)-hexyl]-methyl amine (0.075 g, 0.312 mmol) in anhydrous methanol (1-1.5 mL), and sonicate the mixture for 30 minutes. Remove the solvent in vacuo, add diethyl ether to the residue, filter, wash the solid with diethyl ether, and dry the solid in a vacuum oven (40° C.) overnight to give (S)-[3-(3-chloro-phenoxy)-hexyl]-methyl-amine hydrochloride (0.061 g, 70%). Mass spectrum (ion spray): m/z=242 (M+1), HPLC Method: Xterra RP 18 (4.6×150 mm), elute with a linear gradient of 90/10 through 50/50 (0.1% TFA in water /0.1% TFA in acetonitrile) over 15 min, 1 mL/min, λ=all nm. HPLC Method: purity: 99%, retention time: 10.17 min.

A method similar to that described in Example 55 is used to prepare the following compounds:

| | | | HPLC | |
|---|---|---|---|---|
| | | | Xterra RP 18 | |
| | | | (4.6 × 150 mm), | |
| | | | 90/10 to 50/50 (0.1% | |
| | | Mass | TFA in water/0.1% | |
| | | Spectrum | TFA in | |
| | | (ion spray) | acetonitrile λ = all nm. | |
| | | m/z | | Retention |
| Example | Name | (M + 1) | Purity (%) | Time (min) |
| 56 | (S)-[3-(3-Chloro-4-fluoro-phenoxy)- | 260 | >98 | 10.66 |
| | hexyl]-methyl-amine hydrochloride | | | |
| 57 | (S)-[3-(4-Chloro-3-trifluoromethyl- | 310 | >98 | 11.91 |
| | phenoxy)-hexyl]-methyl-amine | | | |
| | hydrochloride | | | |
| 58 | (S)-[3-(2-Chloro-4-fluoro-phenoxy)- | 260 | 99 | 10.89 |
| | hexyl]-methyl-amine hydrochloride | | | |
| 59 | (S)-[3-(2-Chloro-4-trifluoromethyl- | 310 | 99 | 11.79 |
| | phenoxy)-hexyl]-methyl amine | | | |
| | hydrochloride | | | |
| 60 | (S)-[3-(4-Fluoro-naphthalen-1- | 276 | 98 | 11.75 |
| | yloxy)-hexyl]-methyl-amine | | | |
| | hydrochloride | | | |
| 61 | (S)-[3-(2,3-Difluoro-4-methyl- | 258 | >98 | 10.54 |
| | phenoxy)-hexyl]-methyl-amine | | | |
| | hydrochloride | | | |
| 62 | (R)-[3-(4-Fluoro-naphthalen-1- | 276 | 99 | 11.41 |
| | yloxy)-hexyl]-methyl-amine | | | |
| | hydrochloride | | | |
| 63 | [3-(2,4-Dichloro-phenoxy)-6,6,6- | 330 | 99 | 11.28 |
| | trifluoro-hexyl]-methyl-amine | | | |
| | hydrochloride Enantiomer 1 | | | |
| 64 | [3-(2,4-Dichloro-phenoxy)-4- | 276 | >99 | 10.85 |
| | methyl-penty]-methyl-amine | | | |
| | hydrochloride Enantiomer 1 | | | |
| 65 | [3-(2,4-Dichloro-phenoxy)-5,5- | 304 | >99 | 12.27 |
| | dimethyl-hexyl]-methyl-amine | | | |
| | hydrochloride | | | |
| 66 | [4-Cyclopropyl-3-(2,4-dichloro- | 288 | >99 | 11.18 |
| | phenoxy)-butyl]-methyl-amine | | | |
| | hydrochloride | | | |
| | | HPLC Method | | |
| | | (95/5 to 5/95 0.1% | | |
| | | trifluoroacetic acid in | | |
| | | water/0.1% trifluoroacetic acid in | | |
| | | acetonitrile) Xterra MS | | |
| | | C₁₈ 4.6 mm × 150 mm × 5 micron | | |
| | | | | Retention |
| | | | Purity % | Time |
| 67 | (R)-[3-(2,3,4)-Trichloro- | 312 | 100 | 10.8 |
| | phenoxy)-hexyl]-methyl-amine | | | |
| | hydrochloride | | | |
| 68 | (R)-[3-(3,4,5)-Trichloro- | 312 | 100 | 11.0 |
| | phenoxy)-hexyl]-methyl-amine | | | |
| | hydrochloride | | | |
| 69 | (S)-[3-(2,3,4)-Trichloro- | 312 | 100 | 10.8 |
| | phenoxy)-hexyl]-methyl-amine | | | |
| | hydrochloride | | | |
| 70 | (S)-[3-(3,4,5)-Trichloro- | 312 | 100 | 11.1 |
| | phenoxy)-hexyl]-methyl-amine | | | |
| | hydrochloride | | | |
| | | | HPLC Method | |
| | | | (100/0 to 5/95 | |
| | | | 0.2% formic | |
| | | | acid in | |
| | | | water/0.2% formic | |
| | | | acid in | |
| | | | acetonitrile) Xterra | |
| | | | MS C₁₈ 2.1 mm × | |
| | | | 50 mm × 3.5 micron | |
| | | | | Retention |
| | | | Purity (%) | Time (min) |
| 71 | (S)-[3-(2-Trifluoromethyl-phenoxy)- | 276 | 100 | 2.38 |
| | hexyl]-methyl-amine hydrochloride | | | |
| 72 | (S)-[3-(4-Trifluoromethyl-phenoxy)- | 276 | 100 | 2.39 |
| | hexyl]-methyl-amine hydrochloride | | | |
| 73 | (S)-[3-(2-Chloro-phenoxy)-hexyl]- | 242 | 95 | 2.11 |
| | methyl-amine hydrochloride | | | |
| 74 | (S)-[3-(4-Chloro-phenoxy)-hexyl]- | 244 | 100 | 2.201 |
| | methyl-amine hydrochoride | | | |
| 75 | (S)-[3-(2,3-Dichloro-phenoxy)- | 276 | 100 | 2.36 |
| | hexyl]-methyl-amine hydrochloride | | | |
| 76 | (S)-[3-(Naphthalen-2-yloxy)-hexyl]- | | 100 | 2.39 |
| | methyl-amine hydrochloride | | | |
| 77 | (S)-[3-(Naphthalen-1-yloxy)-hexyl]- | | 100 | 2.39 |
| | methyl-amine | | | |
| 78 | (S)-[3-(2-Chloro-3-trifluoromethyl- | 310 | 100 | 2.53 |
| | phenoxy)-hexyl]-methyl-amine | | | |
| | hydrochloride | | | |
| 79 | (S)-[3-(2,3,5-Trichloro-phenoxy)- | 312 | 100 | 2.69 |
| | hexyl]-methyl-amine hydrochloride | | | |
| 80 | (R)-[3-(4-Chloro-phenoxy)-hexyl]- | 244 | 100 | 2.208 |
| | methyl-amine hydrochoride | | | |
| 81 | (R)-[3-(2,3-Dichloro-phenoxy)- | 276 | 100 | 2.30 |
| | hexyl]-methyl-amine hydrochloride | | | |
| 82 | (R)-[3-(Naphthalen-2-yloxy)-hexyl]- | N/A | 100 | 2.38 |
| | methyl-amine hydrochloride | | | |
| 83 | (R)-[3-(Naphthalen-1-yloxy)-hexyl]- | N/A | 100 | 2.38 |
| | methyl-amine | | | |
| 84 | (R)-[3-(2-Chloro-3-trifluoromethyl- | 310 | 100 | 2.65 |
| | phenoxy)-hexyl]-methyl-amine | | | |
| | hydrochloride | | | |
| 85 | (R)-[3-(2,3,5-Trichloro-phenoxy)- | 312 | 100 | 2.60 |
| | hexyl]-methyl-amine hydrochloride | | | |
| 86 | [3-(2,4-Dichloro-phenoxy)-butyl]- | 248 | 100 | 1.88 |
| | methyl-amine hydrochloride | | | |
| | Enantiomer 1 | | | |
| 87 | [3-(2,4-Dichloro-phenoxy)-pentyl]- | 262 | 100 | 2.00 |
| | methyl-amine hydrochoride | | | |
| | Enantiomer 1 | | | |
| 88 | (S)-[3-(2,4-Dichloro-phenoxy)- | 276 | 100 | 2.38 |
| | hexyl]-methyl-amine hydrochloride | | | |
| 89 | (S)-[3-(3,4-Dichloro-phenoxy)- | 276 | 100 | 2.41 |
| | hexyl]-methyl-amine hydrochloride | | | |
| 90 | (R)-[3-(3,4-Dichloro-phenoxy)- | 276 | 100 | 2.44 |
| | hexyl]-methyl-amine hydrochloride | | | |
| 91 | (S)-[3-(3,5-Dichloro-phenoxy)- | 276 | 100 | 2.49 |
| | hexyl]-methyl-amine hydrochloride | | | |
| 92 | (R)-[3-(3,5-Dichloro-phenoxy)- | 276 | 100 | 2.49 |
| | hexyl]-methyl-amine hydrochloride | | | |
| 93 | (S)-[3-(2,4-Dichloro-6-methyl- | 290 | 100 | 2.52 |
| | phenoxy)-hexyl]-methyl-amine | | | |
| | hydrochloride | | | |
| 94 | (S)-[3-(4-Chloro-3,5-dimethyl- | 270 | 100 | 2.57 |
| | phenoxy)-hexyl]-methyl-amine | | | |
| | hydrochloride | | | |
| 95 | (R)-[3-(4-Chloro-3,5-dimethyl- | 270 | 100 | 2.57 |
| | phenoxy)-hexyl]-methyl-amine | | | |
| | hydrochloride | | | |
| 96 | [3-(2,4-Dichloro-phenoxy)-6-methyl- | 304 | 100 | 2.70 |
| | heptyl]-methyl-amine hydrochloride | | | |
| | Enantiomer 2 | | | |
| 97 | [3-(2,3-Dichloro-phenoxy)-6-methyl- | 304 | 100 | 2.72 |
| | heptyl]-methyl-amine hydrochloride | | | |
| | Enantiomer 1 | | | |
| 98 | [3-(2,3-Dichloro-phenoxy)-6-methyl- | 304 | 95 | 2.73 |
| | heptyl]-methyl-amine hydrochloride | | | |
| | Enantiomer 2 | | | |
| 99 | (R)-[3-(2,4-Dichloro-phenoxy)-4- | 320 | 100 | 2.63 |
| | isobutoxy-butyl]-methyl-amine | | | |
| | hydrochloride | | | |
| 100 | (R)-[3-(2,4-Dichloro-phenoxy)-4- | 306 | 100 | 2.38 |
| | isopropoxy-butyl]-methyl-amine | | | |
| | hydrochloride | | | |
| 101 | (R)-[3-(2,4-Dichloro-phenoxy)-4- | 322 | 100 | 2.53 |
| | isopropylsylfanyl-butyl]-methyl- | | | |
| | amine hydrochloride | | | |
| 102 | (S)-[3-(4-Chloro-2-trifluoromethyl- | 310 | 100 | 2.64 |
| | phenoxy)-hexyl]-methyl-amine | | | |
| | hydrochloride | | | |
| 103 | (R)-[3-(2,4-Dichloro-phenoxy)-4- | 278 | 100 | 1.90 |
| | methoxy-butyl]-methyl-amine | | | |
| | hydrochloride | | | |
| 104 | (R)-[3-(2,4-Dichloro-phenoxy)-4- | 292 | 100 | 2.14 |
| | ethoxy-butyl]-methyl-amine | | | |
| | hydrochloride | | | |
| 105 | (R)-[3-(2,4-Dichloro-phenoxy)-4- | 306 | 100 | 2.38 |
| | isopropoxy-butyl]-methyl-amine | | | |
| | hydrochloride | | | |

### Example 106

### (R)-[3-(2,4-Dichloro-phenoxy)-4-pyrrolidin-1-yl-butyl]-methyl-amine succinate

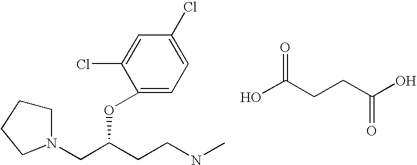

Add succinic acid (0.035 g, 0.003 mmol) to a solution of (R)-[3-(2,4-dichloro-phenoxy)-4-pyrrolidin-1-yl-butyl]-methyl-amine (0.095 g, 0.003 mmol) in anhydrous methanol (5 mL). Stir the reaction mixture for 1 hour, and then concentrate in vacuo. Dry the acquired solid in a heated vacuum oven at 45° C. overnight to obtain (R)-[3-(2,4-dichloro-phenoxy)-4-morpholine-4-yl-butyl]-methyl-amine succinate (0.129 g, 99%). Mass spectrum (ion spray): m/z 317 (M+1), ¹H NMR (CD₃OD) δ7.45 (d, 1H), 7.31 (dd, 1H), 7.24 (d, 1H), 4.89-4.85 (m, 4H), 3.19-2.86 (m, 8H), 2.66 (s, 3H), 2.47 (s, 4H), 2.24-2.08 (m, 2H), 1.91-1.84 (m, 4H).

### Example 107

### (R)-[3-(2,4-Dichloro-phenoxy)-4-morpholine-4-yl-butyl]-methyl-amine succinate

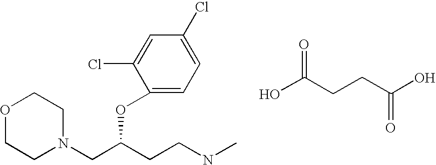

Add succinic acid (0.045 g, 0.0038 mmol) to a solution of (R)-[3-(2,4-dichloro-phenoxy)-4-morpholine-4-yl-butyl]-methyl-amine (0.127 g, 0.0038 mmol) in anhydrous methanol (8 mL). Stir the reaction mixture for 1 hour, and then concentrate in vacuo. Dry the acquired solid in a heated vacuum oven at 45° C. overnight to obtain (R)-[3-(2,4-dichloro-phenoxy)-4-morpholine-4-yl-butyl]-methyl-amine succinate (0.170 g, 99%). Mass spectrum (ion spray): m/z 333 (M+1), ¹H NMR (CD₃OD) δ0.42 (d, 1H), 7.28 (dd, 1H), 7.20 (d, 1H), 4.86 (s, 3H), 4.73-4.68 (m, 1H), 3.61-3.56 (m, 4H), 3.24-3.11 (m, 2H), 2.70 (s, 3H), 2.68-2.50 (m, 6H), 2.50 (s, 4H), 2.23-2.27 (m, 2H).

### Example 108

### (R)-[4-tert-Butoxy-3-(2,4-dichloro-phenoxy)-butyl]-methyl-amine tlifluoroacetate

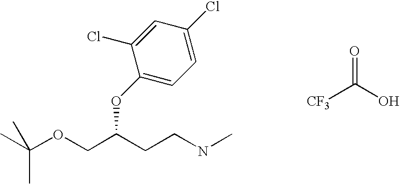

Prepare the HCl salt of (R)-[4-tert-butoxy-3-(2,4-dichloro-phenoxy)-butyl]-methyl-amine in a similar manner to Example 55. Purify the hydrochloride salt on a prep HPLC eluting with 90/0.1/10 to 50/0.1/50 water/TFA/acetonitrile over 20 minutes to give (R)-[4-tert-butoxy-3-(2,4-dichloro-phenoxy)-butyl]-methyl-amine trifluoroacetate (0.030 g, 32%). Mass spectrum (ion spray): m/z=320 (M+1), ¹H NMR (CD₃OD) δ 7.43 (d, 1H), 7.29-7.20 (m, 2H), 4.64-4.60 (m, 1H), 3.59 (d, 2H), 3.28-3.20 (m, 2H), 2.73 (s, 3H), 2.22-2.16 (m, 2H), 1.16 (s, 9H), HPLC Method: Xterra RP 18 (4.6×150 mm), elute with a linear gradient of 90/10 through 50/50 (0.1% TFA in water/0.1% TFA in acetonitrile) over 15 min, 1 mL/min, λ=all nm. HPLC Method: purity: >95%, retention time: 11.49 min.

A method similar to that described in Example 108 is used to prepare the following compounds:

| | | | HPLC | |
|---|---|---|---|---|
| | | | Xterra RP 18 | |
| | | | (4.6 × 150 mm), 90/10 to | |
| | | Mass | 50/50 (0.1% | |
| | | Spectrum | TFA in water/0.1% TFA in | |
| | | (ion spray) | acetonitrile) λ = all nm. | |
| | | m/z | Purity | Retention |
| Example | Name | (M + 1) | (%) | Time (minutes) |
| 109 | (S)-[4-tert-butoxy-3- | 320 | 95 | 11.68 |
| | (2,4-dichloro- | | | |
| | phenoxy)-butyl]- | | | |
| | methyl-amine | | | |
| | trifluoroacetate | | | |

The compounds of the present invention may be used as medicaments in human or veterinary medicine. The compounds may be administered by various routes, for example, by oral or rectal routes, topically or parenterally, for example by injection, and are usually employed in the form of a pharmaceutical composition.

Such compositions may be prepared by methods well known in the pharmaceutical art and normally comprise at least one active compound in association with a pharmaceutically acceptable diluent or carrier. In making the compositions of the present invention, the active ingredient will usually be mixed with a carrier or diluted by a carrier, and/or enclosed within a carrier which may, for example, be in the form of a capsule, sachet, paper or other container. Where the carrier serves as a diluent, it may be solid, semi-solid, or liquid material which acts as a vehicle, excipient or medium for the active ingredient. Thus, the composition may be in the form of tablets, lozenges, sachets, cachets, elixirs, suspensions, solutions, syrups, aerosol (as a solid or in a liquid medium), ointments containing, for example, up to 10% by weight of the active compound, soft and hard gelatin capsules, suppositories, injection solutions and suspensions and sterile packaged powders.

Some examples of suitable carriers are lactose, dextrose, vegetable oils, benzyl alcohols, alkylene glycols, polyethylene glycols, glycerol triacetate, gelatin, carbohydrates such as starch and petroleum jelly, sucrose sorbitol, mannitol, starches, gum acacia, calcium phosphate, alginates, tragacanth, gelatin, syrup, methyl cellulose, methyl- and propyl-hydrobenzoate, talc, magnesium stearate and mineral oil. The compounds of formula (I) can also be lyophilized and the lyophilizates obtained used, for example, for the production of injection preparations. The preparations indicated can be sterilized and/or can contain auxiliaries such as lubricants, preservatives, stabilizers and/or wetting agents, emulsifiers, salts for affecting the osmotic pressure, buffer substances, colourants, flavourings and/or one or more further active compounds, e.g. one or more vitamins. Compositions of the invention may be formulated so as to provide, quick, sustained or delayed release of the active ingredient after administration to the patient by employing procedures well known in the art.

The compositions are preferably formulated in a unit dosage form, each dosage containing from about 5 to about 500 mg, more usually about 25 to about 300 mg, of the active ingredient. The term “unit dosage form” refers to physically discrete units suitable as unitary doses for human subjects and other mammals, each unit containing a predetermined quantity of active material calculated to produce the desired therapeutic effect, in association with a suitable pharmaceutical carrier.

The pharmacological profile of the present compounds may be demonstrated as follows. All of the exemplified compounds above have been found to exhibit Kᵢ values less than 1 μM at the serotonin and norepinephrine transporters as determined using the scintillation proximity assays described below. Preferred compounds typically exhibit a Kᵢ value less than 100 nM at the serotonin transporter and/or a Kᵢ value less than 100 nM at the norepinephrine transporter as determined using the scintillation proximity assays described below. Furthermore, preferred compounds typically selectively inhibit the serotonin and norepinephrine transporters relative to the dopamine transporter.

### Generation of Stable Cell-Lines Expressing the Human Dopamine, Norepinephrine and Serotonin Transporters

Standard molecular cloning techniques are used to generate stable cell-lines expressing the human dopamine, norepinephrine and serotonin transporters. The polymerase chain reaction (PCR) is used in order to isolate and amplify each of the three full-length cDNAs from an appropriate cDNA library. Primers for PCR are designed using the following published sequence data:
Human dopamine transporter: GenBank M95167. Reference: Vandenbergh D J, Persico A M and Uhl G R. A human dopamine transporter cDNA predicts reduced glycosylation, displays a novel repetitive element and provides racially-dimorphic TaqI RFLPs. Molecular Brain Research (1992) volume 15, pages 161-166.
Human norepinephrine transporter: GenBank M65105. Reference: Pacholczyk T, Blakely, R D and Amara S G. Expression cloning of a cocaine-and antidepressant-sensitive human noradrenaline transporter. Nature (1991) volume 350, pages 350-354.
Human serotonin transporter: GenBank L05568. Reference: Ramamoorthy S, Bauman A L, Moore K R, Han H, Yang-Feng T, Chang A S, Ganapathy V and Blakely R D. Antidepressant-and cocaine-sensitive human serotonin transporter: Molecular cloning, expression, and chromosomal localization. Proceedings of the National Academy of Sciences of the USA (1993) volume 90, pages 2542-2546.

The PCR products are cloned into a mammalian expression vector (eg pcDNA3.1 (Invitrogen)) using standard ligation techniques. The constructs are then used to stably transfect HEK293 cells using a commercially available lipofection reagent (Lipofectamine™—Invitrogen) following the manufacture's protocol.

### Scintillation Proximity Assays for Determining the Affinity of Test Ligands at the Norepineohrine and Serotonin Transporters

The compounds of the present invention are norepinephrine and serotonin reuptake inhibitors, and possess excellent activity in, for example, a scintillation proximity assay (e.g. J. Gobel, D. L. Saussy and A. Goetz, J. Pharmacol, Toxicolo. (1999), 42, 237-244). Thus ³H-misoxetine binding to norepinephrine re-uptake sites in a cell line transfected with DNA encoding human norepinephrine transporter binding protein and similarly ³H-citalopram binding to serotonin re-uptake sites in a cell line transfected with DNA encoding human serotonin transporter binding protein are used to determine the affinity of ligands at the norepinephrine and serotonin transporters respectively.

### Norepinephrine Binding Assay

Membrane Preparation:

Cell pastes from large scale production of HEK-293 cells expressing cloned human norepinephrine transporters are homogenized in 4 volumes 50 mM Tris-HCl containing 300 mM NaCl and 5 mM KCl, pH 7.4. The homogenate is centrifuged twice (40,000 g, 10 min, 4° C.) with pellet re-suspension in 4 volumes of Tris-HCl buffer containing the above reagents after the first spin and 8 volumes after the second spin. The suspended homogenate is centrifuged (10 g, 10 min, 4° C.) and the supernatant kept and re-centrifuged (40,000 g, 20 min, 4° C.). The pellet is resuspended in Tris-HCl buffer containing the above reagents along with 10% w/v sucrose and 0.1 mM phenylmethylsulfonyl fluoride (PMSF). The membrane preparation is stored in aliquots (1 ml) at −80° C. until required. The protein concentration of the membrane preparation is determined using a bicinchoninic acid (BCA) protein assay reagent kit (available from Pierce).

[³H]-Nisoxetine Binding Assay:

Each well of a 96 well microtitre plate is set up to contain the following:

| | |
|---|---|
| 50 μl | 2 nM [N-methyl-³H]-Nisoxetine hydrochloride (70-87 Ci/mmol, |
| | from NEN Life Science Products) |
| 75 μl | Assay buffer (50 mM Tris-HCl pH 7.4 containing 300 mM NaCl |
| | and 5 mM KCl) |
| 25 μl | Test compound, assay buffer (total binding) or 10 μM Desipramine |
| | HCl (non-specific binding) |
| 50 μl | Wheatgerm agglutinin coated poly (vinyltoluene) (WGA PVT) |
| | SPA Beads (Amersham Biosciences RPNQ0001) (10 mg/ml) |
| 50 μl | Membrane (0.2 mg protein per ml) |

The microtitre plates are incubated at room temperature for 10 hours prior to reading in a Trilux scintillation counter. The results are analysed using an automatic spline fitting programme (Multicalc, Packard, Milton Keynes, UK) to provide Ki values for each of the test compounds.

### Serotonin Binding Assay

The ability of a test compound to compete with [³H]-citalopram for its binding sites on cloned human serotonin transporter containing membranes are used as a measure of test compound ability to block serotonin uptake via its specific transporter (Ramamoorthy, S., Giovanetti, E., Qian, Y., Blakely, R., (1998) J. Biol. Chem. 273, 2458).

Membrane Preparation:

Membrane preparation is essentially similar to that for the norepinephrine transporter containing membranes as described above. The membrane preparation is stored in aliquots (1 ml) at −70° C. until required. The protein concentration of the membrane preparation is determined using a BCA protein assay reagent kit.

[³H]-Citalopram Binding Assay:

Each well of a 96 well microtitre plate was set up to contain the following:

| | |
|---|---|
| 50 μl | 2 nM [³H]-Citalopram (60-86 Ci/mmol, Amersham |
| | Biosciences) |
| 75 μl | Assay buffer (50 mM Tris-HCl pH 7.4 containing 150 mM |
| | NaCl and 5 mM KCl) |
| 25 μl | Diluted compound, assay buffer (total binding) or 100 μM |
| | Fluoxetine (non-specific binding) |
| 50 μl | WGA PVT SPA Beads (40 mg/ml) |
| 50 μl | Membrane preparation (0.4 mg protein per ml) |

The microtitre plates are incubated at room temperature for 10 hours prior to reading in a Trilux scintillation counter. The results are analysed using an automatic spline fitting programme (Multicalc, Packard, Milton Keynes, UK) to provide Ki (nM) values for each of the test compounds.

### Dopamine Binding Assay

The ability of a test compound to compete with [³H]-WIN35,428 for its binding sites on human cell membranes containing cloned human dopamine transporter are used as a measure of the ability of such test compounds to block dopamine uptake via its specific transporter (Ramamoorthy et al 1998 supra).

Membrane Preparation:

Is essentially the same as for membranes containing cloned human serotonin transporter as described above.

[³H]-WIN35,428 Binding Assay:

Each well of a 96well microtitre plate is set up to contain the following:

| | |
|---|---|
| 50 μl | 4 nM [³H]-WIN35,428 (84-87 Ci/mmol, from NEN Life |
| | Science Products) |
| 75 μl | Assay buffer (50 mM Tris-HCl pH 7.4 containing 150 mM NaCl |
| | and 5 mM KCl) |
| 25 μl | Diluted compound, assay buffer (total binding) or 100 μM |
| | Nomifensine (non-specific binding) |
| 50 μl | WGA PVT SPA Beads (10 mg/ml) |
| 50 μl | Membrane preparation (0.2 mg protein per ml.) |

The microtitre plates are incubated at room temperature for 120 minutes prior to reading in a Trilux scintillation counter. The results are analysed using an automatic spline fitting programme (Multicalc, Packard, Milton Keynes, UK) to provide Ki values for each of the test compounds.

### Formalin Paw Assay

The analgesic effect of compounds of the invention for the treatment of persistent nociceptive pain is demonstrated using the well-known “formalin test.” The formalin test is a model of persistent nociceptive activation induced by tissue injury which can lead to central sensitization. (Shibata, M., Ohkubo, T., Takahashi, H., and Inoki, R., “Modified formalin test: Characteristic biphasic pain response,” Pain (1989) 38: 347-352; and Tjolsen, A., Berge, O. G., Hunskaar, S., Rosland, J. H., and Hole, K., “The formalin test: an evaluation of the method,” Pain (1992) 51:5-17.) The effect of compounds of the invention on formalin-induced paw-licking behavior in the rat is investigated as an index of persistent nociceptive activation. In this test, the injection of formalin under the skin on the dorsal lateral surface of the hind paw of rats causes an immediate and intense increase in the spontaneous activity of C fiber afferents. This activation evokes a distinctly quantifiable behavior indicative of pain, such as licking of the injected paw. The behavioral response to formalin is biphasic, with an early phase that is short lived, followed by an extended tonic response or late phase of persistent nociceptive activation. Mechanisms causing the late phase response, such as central sensitization of pain transmitting neurons, are currently believed to contribute to various types of persistent pains.

Male Sprague-Dawley rats (200-250 g; Charles River, Portage, Mich.) are maintained at constant temperature and light (12 h light/12 h dark) for 4-7 days prior to the studies. Animals have free access to food and water at all times prior to the day of the experiment.

Scoring in the formalin test is performed according to Coderre et al., 1993b and Abbott et al., 1995. (Coderre T. J., Fundytus M. E., McKenna J. E., Dalal S. and Melzack R. “The formalin test: a validation of the weighted-scores method of the behavioral pain rating,” Pain(1993b) 54:

43-50; and Abbott F. V., Franklin K. B. J. and Westbrook R. F. “The formalin test: scoring properties of the first and second phases of the pain response in rats,” Pain (1995) 60: 91-102.) The sum of time spent licking in seconds from time 0 to 5 minutes is considered the early phase while the late phase is taken as the sum of seconds spent licking from 15 to 40 minutes.

Data are presented as means with standard errors of means (±SEM). Data are evaluated by one-way analysis of variance (ANOVA) and the appropriate contrasts analyzed by Tukey's test and Dunnett “t’ test for two-sided comparisons.

The preferred compounds of the present invention may show good stability to the action of the CYP 2D6 enzyme. This is advantageous because it is likely to lead to improved metabolic stability of the compounds.

Stability to the CYP 2D6 enzyme may be determined according to the assay described below:

### In Vitro Determination of the Interaction of Compounds with CYP2D6 in Human Hepatic Microsomes

Cytochrome P450 2D6 (CYP2D6) is a mammalian enzyme which is commonly associated with the metabolism of around 30% of pharmaceutical compounds. Moreover, this enzyme shows a genetic polymorphism with as a consequence a presence in the population of poor and normal metabolizers. A low involvement of CYP2D6 in the metabolism of compounds (i.e. the compound being a poor substrate of CYP2D6) is desirable in order to reduce any variability from subject to subject in the pharmacokinetics of the compound. Also, compounds with a low inhihibitor potential for CYP2D6 are desirable in order to avoid drug-drug interactions with co-administered drugs that are substrates of CYP2D6. Compounds may be tested both as substrates and as inhibitors of this enzyme by means of the following assays.

### CYP2D6 Substrate Assay

Principle:

This assay determines the extent of the CYP2D6 enzyme involvement in the total oxidative metabolism of a compound in microsomes. Preferred compounds of the present invention exhibit less than 75% total metabolism via the CYP2D6 pathway.

For this in vitro assay, the extent of oxidative metabolism in human liver microsomes (HLM) is determined after a 30 minute incubation in the absence and presence of Quinidine, a specific chemical inhibitor of CYP2D6. The difference in the extent of metabolism in absence and presence of the inhibitor indicates the involvement of CYP2D6 in the metabolism of the compound.

Materials and Methods:

Human liver microsomes (mixture of 20 different donors, mixed gender) are acquired from Human Biologics (Scottsdale, Ariz., USA). Quinidine and β NADPH (β Nicotinamide Adenine Dinucleotide Phosphate, reduced form, tetrasodium salt) are purchased from Sigma (St Louis, Mo., USA). All the other reagents and solvents were of analytical grade. A stock solution of the new chemical entity (NCE) is prepared in a mixture of Acetonitrile/Water to reach a final concentration of acetonitrile in the incubation below 0.5%.

The microsomal incubation mixture (total volume 0.1 mL) contained the NCE (4 μM), β NADPH (1 mM), microsomal proteins (0.5 mg/mL), and Quinidine (0 or 2 μM) in 100 mM sodium phosphate buffer pH 7.4. The mixture is incubated for 30 minutes at 37° C. in a shaking waterbath. The reaction was terminated by the addition of acetonitrile (75 μL). The samples are vortexed and the denaturated proteins were removed by centrifugation. The amount of NCE in the supernatant is analyzed by liquid chromatography /mass spectrometry (LC/MS) after addition of an internal standard. A sample is also taken at the start of the incubation (t=0), and analysed similarly.

Analysis of the NCE is performed by liquid chromatography /mass spectrometry. Ten μL of diluted samples (20 fold dilution in the mobile phase) are injected onto a Spherisorb CN Column, 5 μM and 2.1 mm×100 mm (Waters corp. Milford, Mass., USA). The mobile phase consisting of a mixture of Solvent A/Solvent B, 30/70 (v/v) is pumped (Alliance 2795, Waters corp. Milford, Mass., USA) through the column at a flow rate of 0.2 ml/minute. Solvent A and Solvent B are a mixture of ammonium formate 5.1 M pH 4.5/methanol in the proportions 95/5 (v/v) and 10/90 (v/v), for solvent A and solvent B, respectively. The NCE and the internal standard are quantified by monitoring their molecular ion using a mass spectrometer ZMD or ZQ (Waters-Micromass corp, Machester, UK) operated in a positive electrospray ionisation.

The extent of CYP2D6 involvement (% of CYP2D6 involvement) is calculated comparing the extent of metabolism in absence and in presence of quinidine in the incubation.

The extent of metabolism without inhibitor (%) is calculated as follows:

$$\frac{\begin{matrix}( NCE response in samples without inhibitor ) time 0 - \\ ( NCE response in samples without inhibitor ) time 30\end{matrix}}{( NCE response in samples without inhibitor ) time 0} \times 100$$

The extent of metabolism with inhibitor (%) is calculated as follows:

$$\frac{\begin{matrix}( NCE response in samples without inhibitor ) time 0 - \\ ( NCE response in samples with inhibitor ) time 30\end{matrix}}{( NCE response in samples without inhibitor ) time 0} \times 100$$

where the NCE response is the area of the NCE divided by the area of the internal standard in the LC/MS analysis chromatogram, time0 and time30 correspond to the 0 and 30 minutes incubation time.

The % of CYP2D6 involvement is calculated as follows:

$$\frac{\begin{matrix}( \% extent of metabolism without inhibitor ) - \\ ( \% extent of metabolism with inhibitor )\end{matrix}}{\% extent of metabolism without inhibitor} \times 100$$

### CYP2D6 Inhibitor Assay

Principle:

The CYP2D6 inhibitor assay evaluates the potential for a compound to inhibit CYP2D6. This is performed by the measurement of the inhibition of the bufuralol 1′-hydroxylase activity by the compound compared to a control. The 1′-hydroxylation of bufuralol is a metabolic reaction specific to CYP2D6. Preferred compounds of the present invention may exhibit an IC₅₀ higher than 6 μM for CYP2D6 activity, the IC₅₀ being the concentration of the compound that gives 50 % of inhibition of the CYP2D6 activity.

Material and methods:

Human liver microsomes (mixture of 20 different donors, mixed gender) are acquired from Human Biologics (Scottsdale, Ariz.). β⁻ NADPH is purchased from Sigma (St Louis, Mo.). Bufuralol was purchased from Ultrafine (Manchester, UK). All the other reagents and solvents were of analytical grade.

Microsomal incubation mixture (total volume 0.1 mL) contained bufuralol 10 μM, β⁻ NADPH (2 mM), microsomal proteins (0.5 mg/mL), and the new chemical entity (NCE) (0, 5, and 25 μM) in 100 mM sodium phosphate buffer pH 7.4. The mixture is incubated in a shaking waterbath at 37° C. for 5 minutes. The reaction is terminated by the addition of methanol (75 μL). The samples are vortexed and the denaturated proteins are removed by centrifugation. The supernatant was analyzed by liquid chromatography connected to a fluorescence detector. The formation of the 1′-hydroxybufuralol is monitored in control samples (0 μM NCE) and in the samples incubated in presence of the NCE. The stock solution of NCE is prepared in a mixture of Acetonitrile/Water to reach a final concentration of acetonitrile in the incubation below 1.0%.

The determination of 1′hydroxybufuralol in the samples is performed by liquid chromatograhy with fluorimetric detection as described below. Twenty five μL samples are injected onto a Chromolith Performance RP-18e column (100 mm×4.6 mm) (Merck KGAa, Darmstadt, Germany). The mobile phase, consisting of a mixture of solvent A and solvent B whose the proportions changed according the following linear gradient, is pumped through the column at a flow rate of 1 ml/min:

| Time (minutes) | Solvent A (%) | Solvent B (%) |
|---|---|---|
| 0 | 65 | 35 |
| 2.0 | 65 | 35 |
| 2.5 | 0 | 100 |
| 5.5 | 0 | 100 |
| 6.0 | 65 | 35 |

Solvent A and Solvent B consisted of a mixture of 0.02 M potassium dihydrogenophosphate buffer pH3/methanol in the proportion 90/10 (v/v) for solvent A and 10/90 (v/v) for solvent B. The run time is 7.5 minutes. Formation of 1′-hydroxybufuralol is monitored by fluorimetric detection with extinction at λ 252 nm and emission at λ 302 nm.

The IC₅₀ of the NCE for CYP2D6 is calculated by the measurement of the percent of inhibition of the formation of the 1′-hydroxybufuralol in presence of the NCE compared to control samples (no NCE) at a known concentration of the NCE.

The percent of inhibition of the formation of the 1 ′-hydroxybufuralol is calculated as follows:

$$\frac{\begin{matrix}( {1}^{′} \text{-} hydroxybufuralol formed without inhibitor ) - \\ ( {1}^{ ′} \text{-} hydroxybufuralol formed with inhibitor )\end{matrix}}{( {1}^{′} \text{-} hydroxybufuralol area formed without inhibitor )} \times 100$$

The IC₅c is calculated from the percent inhibition of the formation of the 1′-hydroxybufuralol as follows (assuming competitive inhibition):

$$\frac{NCE Concentration \times ( 100 - Percent of inhibition )}{Percent of inhibition}$$

The IC₅₀ estimation is assumed valid if inhibition is between 20% and 80% (Moody G C, Griffin S J, Mather A N, McGinnity D F, Riley R J. 1999. Fully automated analysis of activities catalyzed by the major human liver cytochrome P450 (CYP) enzymes: assessment of human CYP inhibition potential. Xenobiotica, 29(1): 53-75).

We Claim:

1. A compound of formula J: 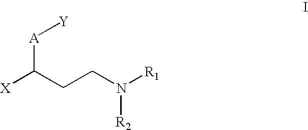
wherein
A is selected from —O— and —S—;
X is selected from C₁-C₈ alkyl, C₂-C₈ alkenyl, and C₄-C₈ cycloalkylalkyl, each of which may be optionally substituted with up to 3 substituents each independently selected from phenyl, pyrrolidinyl, piperidinyl, morpholinyl, halo, C₁-C₄ alkyl, C₁-C₄ alkoxy, C₁-C₄ alkyl-S(O)ₙ— where n is 0, 1 or 2, —CF₃, —CN and —CONH₂;
Y is selected from 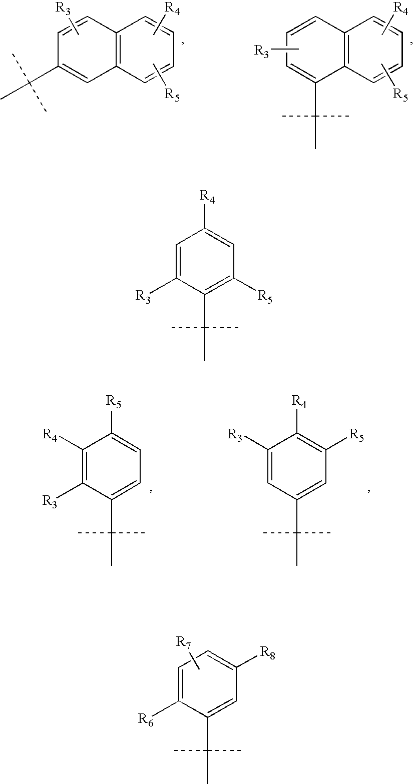
wherein
R₃, R₄ and R₅ are independently selected from hydrogen, halo, C₁-C₄ alkyl, C₁-C₄ alkoxy, C₁-C₄ alkyl-S(O)ₙ— where n is 0, 1 or 2, nitro, acetyl, —CF₃, —SCF₃ and cyano;
R₆ and R₇ are independently selected from halo, C₁-C₄ alkyl, C₁-C₄ alkoxy, C₁-C₄ alkyl-S(O)ₙ— where n is 0, 1 or 2, nitro, acetyl, —CF₃, —SCF₃ and cyano;
R₈ is selected from chloro, bromo, iodo, C₁-C₄ alkyl, C₁-C₄ alkoxy, C₁-C₄ alkyl-S(O)ₙ— where n is 0, 1 or 2, nitro, acetyl, —CF₃, —SCF₃ and cyano; and
R₁ and R₂ are each independently hydrogen or C₁-C₄ alkyl;
or pharmaceutically acceptable salts thereof.

2. A compound as claimed in claim 1, wherein A is —O—.

3. A compound as claimed in claim 1, wherein A is —S—.

4. (canceled)

5. (canceled)

6. A compound as claimed in any one of claims 1-3, wherein the compound possesses the stereochemistry defined in formula II

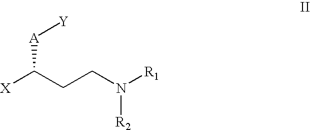

7. A compound as claimed in any one of claims 1-3, wherein the compound possesses the stereochemistry defined in formula III

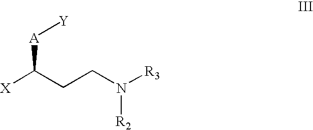

8. A compound as claimed in any one of claims 1 to 3, wherein X is C₁-C₈ alkyl which may be optionally substituted with one substituent independently selected from phenyl, pyrrolidinyl, morpholinyl, halo, C₁-C₄ alkyl, C₁-C₄ alkoxy, C₁-C₄ alkyl-S(O)ₙ— where n is 0, 1 or 2, and —CF₃.

9. (canceled)

10. (canceled)

11. A compound as claimed in any one of claims 1 to 3, wherein X is C₄-C₈ cycloalkylalkyl which may be optionally substituted with up to 3 substituents each independently selected from halo, C₁-C₄ alkyl, C₁-C₄ alkoxy, C₁-C₄ alkyl-S(O)ₙ— where n is 0, 1 or 2, —CF₃, —CN and —CONH₂.

12. (canceled)

13. A compound as claimed in any one of claims 1 to 3, wherein Y is selected from 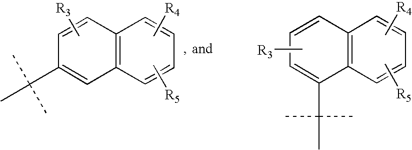
where
R₃, R₄ and R₅ are independently selected from hydrogen, halo, C₁-C₄ alkyl and —CF₃.

14. (canceled)

15. A compound as claimed in any one of claims 1 to 3 wherein Y is selected from 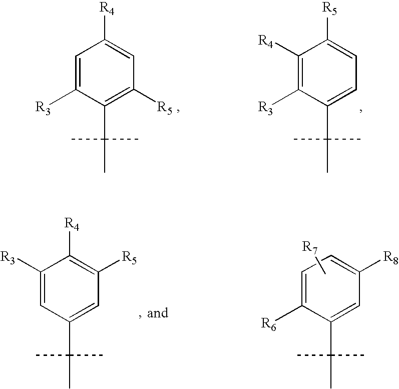
wherein
R₃, R₄ and R₅ are independently selected from hydrogen, halo, C₁-C₄ alkyl, and —CF₃;
R₆ and R₇ are independently selected from halo, C₁-C₄ alkyl, and —CF₃; and
R₈ is selected from chloro, bromo, iodo, C₁-C₄ alkyl, and —CF₃;
provided when R₃ and R₄ are hydrogen, R₅ is not hydrogen.

16. (canceled)

17. (canceled)

18. (canceled)

19. A compound of claim 1 selected from
(S)-Methyl-[3-(2-trifluoromethyl-phenoxy)-hexyl]-amine;
(S)-[3-(3-Chloro-phenoxy)-hexyl]-methyl-amine;
(S)-[3-(3-Chloro-4-fluoro-phenoxy)-hexyl]-methyl-amine;
(S)-[3-(4-Chloro-3-trifluoromethyl-phenoxy)-hexyl]-methyl-amine;
(S)-[3-(2-Chloro-4-fluoro-phenoxy)-hexyl]-methyl-amine;
(S)-[3-(2-Chloro-4-trifluoromethyl-phenoxy)-hexyl]-methyl-amine;
(S)-[3-(4-Fluoro-naphthalen-1-yloxy)-hexyl]-methyl-amine;
(S)-[3-(2,3-Difluoro-4-methyl-phenoxy)-hexyl]-methyl-amine;
(R)-[3-(4-Fluoro-naphthalen-1-yloxy)-hexyl]-methyl-amine;
[3-(2,4-Dichloro-phenoxy)-4-methyl-penty]-methyl-amine;
[3-(2,4-Dichloro-phenoxy)-5,5-dimethyl-hexyl]-methyl-amine;
[4-Cyclopropyl-3-(2,4-dichloro-phenoxy)-butyl]-methyl-amine;
(S)-Methyl-[3-(4-trifluoromethyl-phenoxy)-hexyl]-amine;
(S)-[3-(4-Chloro-phenoxy)-hexyl]-methyl-amine;
(S)-[3-(2,3-Dichloro-phenoxy)-hexyl]-methyl-amine;
(S)-[3-(Naphthalen-2-yloxy)-hexyl]-methyl-amine;
(S)-[3-(Naphthalen-1-yloxy)-hexyl]-methyl-amine;
(S)-[3-(2-Chloro-3-trifluoromethyl-phenoxy)-hexyl]-methyl-amine;
(S)-[3-(2,3,5-Trichloro-phenoxy)-hexyl]-methyl-amine;
(R)-[3-(4-Chloro-phenoxy)-hexyl]-methyl-amine;
(R)-[3-(2,3-Dichloro-phenoxy)-hexyl]-methyl-amine;
(R)-[3-(Naphthalen-2-yloxy)-hexyl]-methyl-amine;
(R)-[3-(Naphthalen-1-yloxy)-hexyl]-methyl-amine;
(R)-[3-(2-Chloro-3-trifluoromethyl-phenoxy)-hexyl]-methyl-amine;
(R)-[3-(2,3,5-Trichloro-phenoxy)-hexyl]-methyl-amine;
[3-(2,4-Dichloro-phenoxy)-butyl]-methyl-amine;
[3-(2,4-Dichloro-phenoxy)-pentyl]-methyl-amine;
(S)-[3-(2,4-Dichloro-phenoxy)-hexyl]-methyl-amine;
(S)-[3-(3,4-Dichloro-phenoxy)-hexyl]-methyl-amine;
(R)-[3-(3,4-Dichloro-phenoxy)-hexyl]-methyl-amine;
(S)-[3-(3,5-Dichloro-phenoxy)-hexyl]-methyl-amine;
(R)-[3-(3,5-Dichloro-phenoxy)-hexyl]-methyl-amine;
(S)-[3-(2,4-Dichloro-6-methyl-phenoxy)-hexyl]-methyl-amine;
(S)-[3-(4-Chloro-3,5-dimethyl-phenoxy)-hexyl]-methyl-amine;
(R)-[3-(4-Chloro-3,5-dimethyl-phenoxy)-hexyl]-methyl-amine;
[3-(2,4-Dichloro-phenoxy)-6-methyl-heptyl]-methyl-amine;
(R)-[3-(2,4-Dichloro-phenoxy)-4-methoxy-butyl]-methyl-amine;
(R)-[3-(2,4-Dichloro-phenoxy)-4-ethoxy-butyl]-methyl-amine;
[3-(2,3-Dichloro-phenoxy)-6-methyl-heptyl]-methyl-amine;
(S)-[3-(4-Chloro-2-trifluoromethyl-phenoxy)-hexyl]-methyl-amine;
(R)-[3-(2,4-Dichloro-phenoxy)-4-isobutoxy-butyl]-methyl-amine;
(R)-[3-(2,4-Dichloro-phenoxy)-4-isopropoxy-butyl]-methyl-amine;
(R)-[3-(2,4-Dichloro-phenoxy)-4-isopropylsylfanyl-butyl]-methyl-amine;
(R)-[4-tert-Butoxy-3-(2,4-dichloro-phenoxy)-butyl]-methyl-amine;
(S)-[4-tert-butoxy-3-(2,4-dichloro-phenoxy)-butyl]-methyl-amine;
(R)-[3-(2,3,4-trichloro-phenoxy)-hexyl]-methyl-amine;
(S)-[3-(2,3,4-trichloro-phenoxy)-hexyl]-methyl-amine;
(R)-[3-(3,4,5-trichloro-phenoxy)-hexyl]-methyl-amine;
(S)-[3-(3,4,5-trichloro-phenoxy)-hexyl]-methyl-amine;
(R)-[3-(2,4-Dichloro-phenoxy)-4-morpholin-4-yl-butyl]-methyl-amine;
(R)-[3-(2,4-Dichloro-phenoxy)-4-pyrrolidin-1-yl-butyl]-methyl-amine;
(S)-[3-(3-Chloro-phenoxy)-hexyl]-methyl-amine hydrochloride;
(S)-[3-(3-Chloro-4-fluoro-phenoxy)-hexyl]-methyl-amine hydrochloride;
(S)-[3-(4-Chloro-3-trifluoromethyl-phenoxy)-hexyl]-methyl-amine hydrochloride;
(S)-[3-(2-Chloro-4-fluoro-phenoxy)-hexyl]-methyl-amine hydrochloride;
(S)-[3-(2-Chloro-4-trifluoromethyl-phenoxy)-hexyl]-methyl amine hydrochloride;
(S)-[3-(4-Fluoro-naphthalen-1-yloxy)-hexyl]-methyl-amine hydrochloride;
(S)-[3-(2,3-Difluoro-4-methyl-phenoxy)-hexyl]-methyl-amine hydrochloride;
(R)-[3-(4-Fluoro-naphthalen-1-yloxy)-hexyl]-methyl-amine hydrochloride;
[3-(2,4-Dichloro-phenoxy)-6,6,6-trifluoro-hexyl]-methyl-amine hydrochloride;
[3-(2,4-Dichloro-phenoxy)-4-methyl-penty]-methyl-amine hydrochloride;
[3-(2,4-Dichloro-phenoxy)-5,5-dimethyl-hexyl]-methyl-amine hydrochloride;
[4-Cyclopropyl-3-(2,4-dichloro-phenoxy)-butyl]-methyl-amine hydrochloride;
(R)-[3-(2,3,4)-Trichloro -phenoxy)-hexyl]-methyl-amine hydrochloride;
(R)-[3-(3,4,5)-Trichloro -phenoxy)-hexyl]-methyl-amine hydrochloride;
(S)-[3-(2,3,4)-Trichloro -phenoxy)-hexyl]-methyl-amine hydrochloride;
(S)-[3-(3,4,5)-Trichloro -phenoxy)-hexyl]-methyl-amine hydrochloride;
(S)-[3-(2-Trifluoromethyl-phenoxy)-hexyl]-methyl-amine hydrochloride;
(S)-[3-(4-Trifluoromethyl-phenoxy)-hexyl]-methyl-amine hydrochloride;
(S)-[3-(2-Chloro-phenoxy)-hexyl]-methyl-amine hydrochloride;
(S)-[3-(4-Chloro-phenoxy)-hexyl]-methyl-amine hydrochoride;
(S)-[3-(2,3-Dichloro-phenoxy)-hexyl]-methyl-amine hydrochloride;
(S)-[3-(Naphthalen-2-yloxy)-hexyl]-methyl-amine hydrochloride;
(S)-[3-(Naphthalen-1-yloxy)-hexyl]-methyl-amine hydrochloride;
(S)-[3-(2-Chloro-3-trifluoromethyl-phenoxy)-hexyl]-methyl-amine hydrochloride;
(S)-[3-(2,3,5-Trichloro-phenoxy)-hexyl]-methyl-amine hydrochloride;
(R)-[3-(4-Chloro-phenoxy)-hexyl]-methyl-amine hydrochloride;
(R)-[3-(2,3-Dichloro-phenoxy)-hexyl]-methyl-amine hydrochloride;
(R)-[3-(Naphthalen-2-yloxy)-hexyl]-methyl-amine hydrochloride;
(R)-[3-(Naphthalen-1-yloxy)-hexyl]-methyl-amine hydrochloride;
(R)-[3-(2-Chloro-3-trifluoromethyl-phenoxy)-hexyl]-methyl-amine hydrochloride;
(R)-[3-(2,3,5-Trichloro-phenoxy)-hexyl]-methyl-amine hydrochloride;
[3-(2,4-Dichloro-phenoxy)-butyl]-methyl-amine hydrochloride;
[3-(2,4-Dichloro-phenoxy)-pentyl]-methyl-amine hydrochloride;
(S)-[3-(2,4-Dichloro-phenoxy)-hexyl]-methyl-amine hydrochloride;
(S)-[3-(3,4-Dichloro-phenoxy)-hexyl]-methyl-amine hydrochloride;
(R)-[3-(3,4-Dichloro-phenoxy)-hexyl]-methyl-amine hydrochloride;
(S)-[3-(3,5-Dichloro-phenoxy)-hexyl]-methyl-amine hydrochloride;
(R)-[3-(3,5-Dichloro-phenoxy)-hexyl]-methyl-amine hydrochloride;
(S)-[3-(2,4-Dichloro-6-methyl-phenoxy)-hexyl]-methyl-amine hydrochloride;
(S)-[3-(4-Chloro-3,5-dimethyl-phenoxy)-hexyl]-methyl-amine hydrochloride;
(R)-[3-(4-Chloro-3,5-dimethyl-phenoxy)-hexyl]-methyl-amine hydrochloride;
[3-(2,4-Dichloro-phenoxy)-6-methyl-heptyl]-methyl-amine hydrochloride;
[3-(2,3-Dichloro-phenoxy)-6-methyl-heptyl]-methyl-amine hydrochloride;
[3-(2,3-Dichloro-phenoxy)-6-methyl-heptyl]-methyl-amine hydrochloride;
(R)-[3-(2,4-Dichloro-phenoxy)-4-isobutoxy-butyl]-methyl-amine hydrochloride;
(R)-[3-(2,4-Dichloro-phenoxy)-4-isopropoxy-butyl]-methyl-amine hydrochloride;
(R)-[3-(2,4-Dichloro-phenoxy)-4-isopropylsylfanyl-butyl]-methyl-amine hydrochloride;
(S)-[3-(4-Chloro-2-trifluoromethyl-phenoxy)-hexyl]-methyl-amine hydrochloride;
(R)-[3-(2,4-Dichloro-phenoxy)-4-methoxy-butyl]-methyl-amine hydrochloride;
(R)-[3-(2,4-Dichloro-phenoxy)-4-ethoxy-butyl]-methyl-amine hydrochloride;
(R)-[3-(2,4-Dichloro-phenoxy)-4-isopropoxy-butyl]-methyl-amine hydrochloride;
(R)-[3-(2,4-Dichloro-phenoxy)-4-pyrrolidin-1-yl-butyl]-methyl-amine succinate;
(R)-[3-(2,4-Dichloro-phenoxy)-4-morpholine-4-yl-butyl]-methyl-amine succinate;
(R)-[4-tert-Butoxy-3-(2,4-dichloro-phenoxy)-butyl]-methyl-amine trifluoroacetate; and
(S)-[4-tert-Butoxy-3-(2,4-dichloro-phenoxy)-butyl]-methyl-amine trifluoroacetate.

20. (canceled)

21. A pharmaceutical composition comprising a compound of formula I or a pharmaceutically acceptable salt thereof, as defined in any one of claims 1 or 19, together with a pharmaceutically acceptable diluent or carrier.

22. (canceled)

23. (canceled)

24. (canceled)

25. (canceled)

26. (canceled)

27. (canceled)

28. (canceled)

29. (canceled)

30. (canceled)

31. A method for treating a disorder selected from depression OCD, anxiety memory loss, urinary incontinence conduct disorders ADHD, obesity, alcoholism, smoking cessation, hot flashes/flushes or pain in mammals, comprising administering to a mammal patient in need thereof an effective amount of a compound of formula I or a pharmaceutically acceptable salt thereof, as defined in any one of claims 1 or 19.

32. A method as claimed in claim 31, where the mammal is human.
